(19) United States
(12) Patent Application Publication
(10) Pub. No.: US 2015/0119447 A1
(43) Pub. Date: Apr. 30, 2015

(54) CLUSTERED SINGLE NUCLEOTIDE POLYMORPHISMS IN THE HUMAN ACETYLCHOLINESTERASE GENE AND USES THEREOF IN DIAGNOSIS AND THERAPY

(71) Applicant: YISSUM RESEARCH DEVELOPMENT COMPANY OF THE HEBREW UNIVERSITY OF JERUSALEM LTD., Jerusalem (IL)

(72) Inventors: Hermona Soreq, Jerusalem (IL); Ella Sklan, Raanana (IL); Shani Shenhar-Tsarfaty, Jordan Valley (IL); Geula Hanin, Jerusalem (IL)

(21) Appl. No.: 14/399,612

(22) PCT Filed: May 9, 2013
(86) PCT No.: PCT/IL2013/050398

§ 371 Date: Nov. 7, 2014

Related U.S. Application Data
(60) Provisional application No. 61/644,840, filed on May 9, 2012.

(51) Int. Cl. C12Q 1/68; A61K 31/7105

(52) U.S. Cl. CPC C12Q 1/6883; A61K 31/7105; C12Q 2600/156; C12Q 2600/178; C12Q 2600/118; C12Q 2600/106; C12Q 2600/172
USPC 514/44 A; 435/6.11; 506/9; 506/16; 506/2

(57) ABSTRACT

The present invention relates to diagnostic and prognostic methods and kits for determining genetic predisposition for at least one AChE-associated disorder, as well for diagnosing, prognosing and monitoring said disorders. The methods of the invention are based on detection of specific SNPs that modulate the interaction of different miRNAs to AChE 3′-UTR.

### TECHNOLOGICAL FIELD

The invention relates to diagnostic methods for determining genetic predisposition for disorders associated with acetylcholinesterase (AChE) signaling. More particularly, the invention provides methods and kits for detecting single nucleotide polymorphisms (SNPs) in the 3′-UTR of the AChE gene that modulate microRNA regulation of the AChE signaling.

### PRIOR ART

References considered to be relevant as background to the presently disclosed subject matter are listed below:
[1] Shapira, M. et al. Hum Mol Genet. 9:1273-81 (2000);
[2] Soreq, H. and Seidman S. Nat Rev Neurosci. 9:670 (2001);
[3] Filipowicz, W. et al. Nature reviews. Genetics 9:102-114 (2008);
[4] Bartel, D. P. Cell 136:215-233 (2009);
[5] Lau, P. & de Strooper, B. Seminars in cell & developmental biology 21:768-773 (2010);
[6] Abelson, J. F. et al. Science 310:317-320 (2005);
[7] Martin, M. M. et al. The Journal of biological chemistry 282:24262-24269 (2007);
[8] Sklan, E. H. et al. PNAS 101:5512-5517 (2004);
[9] Landegren, U. et al., Genome Research 769(8):769-776 (1998);
[10] Shi, M. M. Clinical Chemistry 47(2):164-172 (2001);
[11] Albano F. et al. Journal of Hematology & Oncology 5:48 (2012);
[12] Boissonneault, V. et al. JBC 284(4):1971-1981 (2009);
[13] Robson, J. E. et al. RNA 18:135-144 (2012);]
[14] Elmen, J. et al. Nature 452:896-899 (2008);
[15] Xiaojuan, C. et al. Int. J. Mol. Sci. 14:7089-7108 (2013);
[16] Ellman, G. L., et al., Biochem. Pharmacol. 7:88-95 (1961);
[17] Pohanka, M. et al. Int. J. Mol. Sci. 12:2631-2640 (2011);
[18] Birman, S. Biochem. J. 225:825-828 (1985);
[19] Ofek, K. et al. J Mol Med (Berl) 85:1239-1251 (2007);
[20] Hasin, Y. et al. Human mutation 24:408-416 (2004);
[21] Hanin, G. & Soreq, H. Frontiers in molecular neuroscience 4:28 (2011);
[22] Benmoyal-Segal, L. Faseb J 19:452-454 (2005);
[23] Browne, R. 0. et al. Faseb J 20:1733-1735 (2006);
[24] Loewenstein-Lichtenstein, Y. et al. Nat Med 1:1082-1085 (1995);
[25] Neville, L. F. et al. J Neurosci Res 27:452-460 (1990);
[26] Podoly, E. et al. J Biol Chem 284:17170-17179 (2009);
[27] Velan, B. et al. Cellular and molecular neurobiology 11:143-156 (1991);
[28] Homola, J. Chemical reviews 108:462-493 (2008);
[29] Jeyapalan, Z. et al. Nucleic acids research 39:3026-3041 (2011);
[30] Kang, J. G. et al. The Journal of pathology 225:378-389 (2011);
[31] Papadopoulos, T. et al. The EMBO journal 26:3888-3899 (2007);
[32] Vallieres, L. et al. The Journal of neuroscience: the official journal of the Society for Neuroscience 22:486-492 (2002);
[33] Berson, A. et al. EMBO molecular medicine 4:730-742 (2012);
[34] Prody et al. PNAS 84:3555-3559 (1987);
[35] Soreq et al. PNAS 24:9688-92 (1990);
[36] Kaufer, D. et al. Nature 393:373-377 (1998);
[37] Pavlov, V. A. et al. Brain, behavior, and immunity 23:41-45 (2009);
[38] Tyagarajan, S. K. et al. Journal of cell science 124(2):786-796 (2011);
[39] Guyenet, P. G. Nature reviews. Neuroscience 7:335-346 (2006);
[40] Lettre, G. et al. PLoS genetics 7 e1001300 (2011);
[41] Shaked, et al. Immunity 31:854-855 (2009).

Acknowledgement of the above references herein is not to be inferred as meaning that these are in any way relevant to the patentability of the presently disclosed subject matter.

### BACKGROUND OF THE INVENTION

Stress is known to activate numerous physiological systems in the body and hormones appear to play a pivotal role in translating the stress response in the brain into long-term functional changes in peripheral tissues and organs.

Prolonged stress may be particularly detrimental. For example, early childhood self-reported anxiety and depression predict post-traumatic stress disorder (PTSD) following stress. Nevertheless, to date there are no objective prognostic tools available for large scale screening of trauma-exposed individuals to identify those with inherited susceptibility and allow selection for treatment. Such screening is especially valuable in light of studies suggesting the importance of early treatment; thus, Trauma-Focused early intervention based on cognitive-behavioral treatment was reported to be effective for individuals with traumatic stress symptoms. These results point to the importance of early screening and diagnosis of individuals in high risk for trauma-related psychopathologies.

Cholinergic signaling in general and the ACh hydrolyzing enzyme Acetylcholinesterase (AChE) specifically, is simultaneously involved in central cognitive processes such as learning, memory and stress responses and in activating the parasympathetic system Animal studies and some human data show that AChE, an essential controller of ACh levels, is subject to significant alterations following stress. The AChE extended promoter contains a functional glucocorticoid response element, leading to over-expression under stress. Additionally, alternative splicing replaces the synaptic AChE variant (AChE-S) with a normally rare soluble monomer form-“read-through” AChE (AChE-R). AChE-R may rapidly counteract and attenuate the effects of ACh hyper-secretion [1, 2], providing long-term protection from progressive neural injury Importantly, several Middle-East abundant polymorphisms in the AChE and the homologous butyrylcholinesteras (BChE) gene could affect the ability of individuals to cope with cholinergic challenges. The known polymorphisms and biochemical variability of AChE further affect inherited trait and state anxiety. Additionally, as is reported herein, polymorphisms in the non-coding regions of these genes may affect the capacity of microRNAs (miRs) to regulate cholinesterase levels. MiRs are short non-coding RNAs, 20-25 nucleotides long, that can simultaneously regulate multiple gene functions in a single biological pathway [3] by post-transcriptionally suppressing translation or inducing degradation of their mRNA targets [4, 5]. However, the biological impact of maintaining the balance among multiple miR-target interactions or how impairments in one interaction would affect others has not been well studied.

At the genomic level, SNP interference with miR functions was shown to affect the expression of these miRs' targets, modify higher brain functions and induce a risk of chronic disease. Examples include a 3′-UTR SNP in the brain-expressed human Slit and Trk-like 1 (SLITRK1) gene which strengthens an existing miR-189 target site and is involved in Tourette's syndrome [6], and a 1166A/C SNP in the angiotensin receptor 1 (AGTR1) gene which abrogates its miR-155-mediated regulation, resulting in elevation of the AGTR1 protein which is implicated in hypertension and cardiovascular disease [7]. However, whether any of these phenotypes also reflects mis-regulation of other targets was not addressed.

Hence, exploring SNP-mediated miR malfunctioning in conditions associated with stress and inflammation is desirable, and may provide a powerful diagnostic and therapeutic tool for these conditions.

### SUMMARY OF THE INVENTION

According to a first aspect, the invention relates to a method for the diagnosis of a genetic predisposition of a human subject for at least one AChE-signaling associated disorder, the method is based on genotyping in a biological sample of the tested subject at least one SNP in the 3′UTR of the AChE gene. In specific embodiments, these SNPs modulate binding of different miRNAs to the AChE gene. A second aspect of the invention relates to a method for the diagnosis of an AChE-signaling associated disorder in a human subject based on the presence of said SNPs and the expression of different targets of the specific miRNA. Still further, the invention provides therapeutic methods using agents that reverse the modulatory action of the SNP. In a further aspect, the invention provides a kit for performing the diagnostic methods of the invention.

These and other aspects of the invention will become apparent by the hand of the following drawings.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1D. A Schematic Presentation of Clustered miR-Silencing SNPs in the 3′-UTR of the Human AChE Gene

FIG. 1A. A schematic structure of the human AChE gene.

FIG. 1B. A schematic structure of the human AChE-R transcript.

FIG. 1C. A schematic structure of the human AChE-S transcript. Numbering in FIGS. 1A and 1n FIG. 1B indicates mapping of SNPs location.

FIG. 1D. Nucleotide sequence of miRs targeting the 3′UTR of AChE, AChE/miR binding sites and their respective SNPs. miR seed regions are boxed. The 3′ to 5′ nucleic acid sequences of hsa-miR-4319, -125a-5p, -125b-3p, -4283, -761, -214, -298 and hsa-miR-608 are denoted by SEQ ID NO. 24, SEQ ID NO. 28, SEQ ID NO. 32, SEQ ID NO. 36, SEQ ID NO. 40, SEQ ID NO. 44, SEQ ID NO. 48 and SEQ ID NO. 51, respectively. The 5′ to 3′ nucleic acid sequence of the AChE major allele at the binding site of hsa-miR-4319, -125a-5p, -125b-3p, -4283, -761, -214, -298 and hsa-miR-608 are denoted by SEQ ID NO. 25, SEQ ID NO. 29, SEQ ID NO. 33, SEQ ID NO. 37, SEQ ID NO. 41, SEQ ID NO. 45, SEQ ID NO. 49 and SEQ ID NO. 52, respectively. The 5′ to 3′ nucleic acid sequence of the AChE minor allele at the binding site of hsa-miR-4319, -125a-5p, -125b-3p, -4283, -761, -214, -298 and hsa-miR-608 are denoted by SEQ ID NO. 9, SEQ ID NO. 10, SEQ ID NO. 11, SEQ ID NO. 12, SEQ ID NO. 13, SEQ ID NO. 14, SEQ ID NO. 15 and SEQ ID NO. 16, respectively. Abbreviations: acetylcholinesterase, AChE; read-through AChE variant, AChE-R; synaptic AChE variant, AChE-S; base pair, bp; homo sapiens, hsa; mouse, mmu, chromosome; chr.

FIG. 2A-2F. miR-608 Directly Interacts with the 3′ UTR of Human AChE

FIG. 2A. A schematic presentation of the mechanism by which AChE-miR interactions modify ACh signaling, anxiety and blood pressure.

FIG. 2B. A schematic presentation of synaptic AChE mRNA (AChE-S), with the C2098A SNP in its 3′ UTR region.

FIG. 2C. Nucleic acid sequence of the complementary regions of AChE alleles, miR-608 and miR-132. Seed regions are boxed and the SNP is bold.

The 3′ to 5′ nucleic acid sequences of hsa-miR-608 is denoted by SEQ ID NO. 51, the 5′ to 3′ nucleic acid sequence of the AChE major allele at the binding site of hsa-miR-608 is denoted by SEQ ID NO. 52 and the 5′ to 3′ nucleic acid sequence of the AChE minor allele at the binding site of hsa-miR-608 is denoted by SEQ ID NO. 16. The 3′ to 5′ nucleic acid sequences of hsa-miR-132 is denoted by SEQ ID NO. 59 and the 5′ to 3′ nucleic acid sequence of the AChE major allele at the binding site of hsa-miR-132 is denoted by SEQ ID NO. 60.

FIG. 2D. A bar diagram showing luciferase activity of HEK-293T cells expressing luciferase-AChE 3′UTR and infected with miR-132, miR-608 or control lentiviruses. FIG. 2E. A bar diagram showing AChE activity in human U937 lentivirus-infected U937 cells.

FIG. 2F. A bar diagram showing luciferase activity of HEK-293T cells expressing luciferase-linked major or minor rs17228616 AChE 3′UTR alleles and infected with either miR-608 or control lentiviruses.

Abbreviations: acetylcholine, ACh; acetylcholinesterase, AChE; synaptic AChE variant, AChE-S; Relative luciferase units, RLU; control, cont; acetylcholinesterase 3′ un-translated region, AChE 3′ UTR; miR; micro-RNA.

FIG. 3A-3D. HEK-293T Cells Survival Assay

FIG. 3A. A schematic diagram of the vector used for analyzing binding of miR to the 3′ UTR of AChE.

FIG. 3B. Photographs obtained for HEK-293T cells carrying AChE 3′UTR fused to a cytotoxic sensor, infected with lentiviruses encoding miR-132, -608, or control sequence.

FIG. 3C. A bar diagram showing the copy number of AChE 3′UTR in stable HEK-293T cell lines.

FIG. 3D. A bar diagram showing miR-608 expression in transfected 293T cells with prevalent (major allele, or allele C) or SNP (minor allele, or allele A) AChE 3′UTR. Abbreviations: control, cont; scrambled sequence, scr.

FIG. 4A-4K. Measurements of miR-608/Target Interactions

FIG. 4A. Nucleic acid sequences of target and miR RNA oligonucleotides used for SPR. Seed sequences are bold. The depicted nucleic acid sequence of miR-608, AChE (C allele) binding site to miR-608, AChE (A allele) binding site to miR-608, CDC42 binding site to miR-608, miR-132 and AChE binding site to miR-132 are denoted by SEQ ID NO. 3, SEQ ID NO. 4, SEQ ID NO. 5, SEQ ID NO. 6, SEQ ID NO. 7 and SEQ ID NO. 8, respectively.

FIG. 4B. Schematic presentation of predicted structures and binding energies of miR-608 with AChE's C-allele and A-allele and with CDC42, and of miR-132 with AChE.

FIG. 4C. A SPR sensogram showing binding of miR-608 to the C-allele of AChE.

FIG. 4D. A SPR sensogram showing binding of miR-608 to the A-allele of AChE.

FIG. 4E. A SPR sensogram showing binding of miR-608 to the CDC42 target.

FIG. 4F. A SPR sensogram showing miR-132/AChE binding. Biotinylated target RNA oligonucleotides were immobilized to a streptavidin chip and increasing concentrations (0.3125, 0.625, 1.25, 2.5, 5, 10 μM) of miR oligonucleotides were injected over the chip (C—F).

FIG. 4G. A graph showing SPR dissociation slopes of the indicated interactions.

FIG. 4H. A table summarizing kₐ and k_{d} values for the SPR reactions.

FIG. 4I. Nucleic acid sequences of primers used for site directed mutagenesis to create the minor allele of SNP C2098A AChE 3′UTR sequence. The depicted nucleic acid sequence of the forward primer and of the reverse primer is denoted by SEQ ID NO. 1 and SEQ ID NO. 2, respectively.

FIG. 4J. SPR sensograms showing binding of miR-608 binding to the major allele (allele C) or to the minor allele (allele A) of SNP C2098A in AChE. Results were obtained from duplicate experiments to the experiments presented in FIG. 4C and FIG. 4D, respectively.

FIG. 4K. SPR sensograms showing binding of miR-132 to AChE-R or AChE-S. Abbreviations: response units, R.U.; second, sec.

FIG. 5A-5I. The Minor Rs17228616 Allele Leads to Limited AChE Suppression while Potentiating CDC42 and IL6 Suppression

FIG. 5A. A schematic model suggesting that a weakened miR-608/AChE interaction modifies CDC42 and IL6 suppression by miR-608.

FIG. 5B. A representative immunoblot (left panel) and a quantification thereof (right panel) of CDC42 and GAPDH in HEK-293T cells expressing the two AChE alleles of SNP C2098A, major (allele C) and minor (allele A) and transfected with miR-608 or control lentiviruses. n=3 experiments, each in duplicates or triplicate.

FIG. 5C. Genotyped (DNA) sequences of the two AChE alleles complementary to the major (C) and minor (A) alleles of SNP C2098A in human brain tissues. The nucleic acid sequence of the depicted major allele is denoted by SEQ ID NO. 61 and the nucleic acid sequence of the depicted minor allele is denoted by SEQ ID NO. 62.

FIG. 5D. A bar diagram showing brain miR-608 levels in homozygous samples of both AChE SNP C2098A major and minor alleles.

FIG. 5E. A bar diagram showing levels of AChE activity in brain samples from major and minor AChE SNP C2098A allele homozygotes.

FIG. 5F. A bar diagram showing levels of BChE activity in brain samples from major and minor AChE SNP C2098A allele homozygotes.

FIG. 5G. A representative immunoblot showing levels of IL6, CDC42 and GAPDH (as a control) in brain samples of homozygous for the major or minor allele AChE SNP C2098A.

FIG. 5H. A bar diagram showing quantification of the levels of CDC42 in brain samples of homozygous for the major or minor allele of AChE SNP C2098A.

FIG. 5I. A bar diagram showing quantification of the levels of IL6 in brain samples of homozygous for the major or minor allele of AChE SNP C2098A. Abbreviations: control, cont.

FIG. 6A-6E. Healthy Heterozygotes and Homozygotes of the Minor Rs17228616 Allele Show Reduced Cortisol, and Elevated Blood Pressure

FIG. 6A. Pie diagrams showing the numbers of homozygotes for the major allele and homozygotes and heterozygotes for the minor rs17228616 allele in the HERITAGE cohort; the numbers of homozygotes for the major allele and homozygotes and heterozygotes for the minor rs17228616 allele is shown for Caucasian (left panel, N=196 and 13, respectively) and for African-American (right panel, N=96 and 63, respectively).

FIG. 6B. A diagram showing cortisol levels in heterozygotes and homozygotes for the minor allele of SNP C2098A.

FIG. 6C. A diagram showing systolic blood pressure levels in heterozygotes and homozygotes for the minor allele of SNP C2098A.

FIG. 6D. A diagram showing diastolic blood pressure in heterozygotes and homozygotes for the minor allele of SNP C2098A. The levels in FIG. 6B-6D are shown for sections of the HERITAGE cohort (African-American, indicated by white boxes and Caucasian, indicated by gray boxes) as well as for the combined population (indicated by a diamond) of heterozygotes and homozygotes of the minor allele.

FIG. 6E. A model of miR-608/AChE interaction demonstrating that the major AChE allele (allele C of SNP C2098A) enables balanced AChE, CDC42 and IL6 levels, which together contribute to controlling anxiety and blood pressure. The minor AChE allele (allele A of AChE SNP C2098A) enhances AChE levels and reduces CDC42 and IL6, thereby dually elevating anxiety and blood pressure.

FIG. 7A-7G. Healthy Heterozygotes and Homozygotes of the Minor Allele of C2098A SNP Show Reduced Cortisol, and Blood Pressure

FIG. 7A. A pie diagram showing the numbers of homozygotes for the major allele (N=295) and homozygotes (N=17) and heterozygotes (N=60) for the minor allele of SNP C2098A in the HERITAGE cohort.

FIG. 7B. A bar diagram showing serum levels of C-reactive protein in homozygous of the major allele and in heterozygous and homozygous of the minor allele of SNP C2098A.

FIG. 7C. A bar diagram showing levels of anxiety (Trait and State) in homozygous of the major allele and in heterozygous and homozygous of the minor allele of SNP C2098A.

FIG. 7D. Bar diagrams showing distinct distribution patterns of C-reactive protein (CRP, left panel), trait anxiety (center panel) and state anxiety (right panel) in homozygous of the major allele and in heterozygous and homozygous of the minor allele of SNP C2098A.

FIG. 7E. Bar diagrams showing distinct distribution patterns of serum cortisol levels (left panel), systolic blood pressure (center panel) and diastolic blood pressure (right panel) in homozygous of the major allele and in heterozygous and homozygous of the minor allele of SNP C2098A.

FIG. 7F. A bar diagram showing C-reactive protein level of heterozygous and homozygous of the minor allele compared with homozygous of the major allele of SNP C2098A.

FIG. 7G. Bar diagrams showing STAI scores (Trait and state anxiety, left and right panel, respectively) of heterozygous and homozygous of the minor allele compared with homozygous of the major allele of SNP C2098A.

FIG. 8A-B. Global cognition, memory and executive function in stroke patients homozygous and heterozygous of the minor allele, and homozygous of the major allele of AChE SNP C2098A, at admission and 2 years post stroke.

FIG. 8A. Bar diagrams showing global cognition, memory and executive function in stroke patients homozygous and heterozygous of the minor allele, and homozygous of the major allele of AChE SNP C2098A, at admission.

FIG. 8B. Bar diagrams showing delta computerised of cognitive global scores (Mindstreams cop.) of 2 years minus admission; homozygous and heterozygous for the minor allele of AChE SNP C2098A presented cognitive deterioration and a smaller improvement in memory, while homozygous for the major allele of AChE SNP C2098A re-gain cognitive status, and present a larger improvement in memory.

(Delta=2 year score minus admission, Delta Global cognitive score, p=0.022, delta Memory p=0.032, delta Executive function p=0.065).

FIG. 9A-9D. miR-125b Targets AChE 3′-UTR

FIG. 9A. Nucleotide sequence of miR-125b targeting the 3′UTR of AChE, AChE/miR-125b binding site and the sequences of the complementary AChE alleles of SNP G2165A. Seed regions of the miR binding site are boxed and the SNP is bold. The 3′ to 5′ nucleic acid sequence of the depicted hsa-miR-125b-3p and the 5′ to 3′ nucleic acid sequence of has-miR-125b-3p binding site on AChE major and minor allele are denoted by SEQ ID NO. 32, SEQ ID NO. 33 and SEQ ID NO. 11, respectively. FIG. 9B. A schematic diagram of the psiCHECK-2 vector used for analyzing binding of miRS to the 3′ UTR of AChE (left panel) and a bar diagram showing relative luciferase activity of HEK-293T cells expressing luciferase-AChE 3′UTR and infected with miR-132, miR-125b or control lentiviruses. FIG. 9C. A schematic diagram of the vector used for analyzing binding of miR to the 3′ UTR of AChE (left panel) and photographs of life and death assay obtained for HEK-293T cells carrying AChE 3′UTR fused to a cytotoxic sensor, infected with lentiviruses encoding miR-132, -125b, or control sequence. In case of binding to the 3′UTR the cells survive but in lack of binding cells die. FIG. 9D. AChE activity in human U937 lentivirus-infected cells.

FIG. 10A-10D. AChE 3′-UTR SNPs Disrupting miR-608 and miR-125 Suppression Associate with Elevated Inflammation and Anxiety in HERITAGE Cohort Members

FIG. 10A. A bar diagram showing C-reactive protein level of heterozygous and homozygous of the minor allele compared with homozygous of the major allele (left panels show the miR-608 binding site SNP and the right panels show the miR-125-b binding site SNP).

FIG. 10B. A bar diagram showing TNF protein level of heterozygous and homozygous of the minor allele compared with homozygous of the major allele (left panels show the miR-608 binding site SNP and the right panels show the miR-125-b binding site SNP).

FIG. 10C. A bar diagram showing scores of state anxiety of heterozygous and homozygous of the minor allele compared with homozygous of the major allele (left panels show the miR-608 binding site SNP and the right panels show the miR-125-b binding site SNP).

FIG. 10D. A bar diagram showing scores of trait anxiety of heterozygous and homozygous of the minor allele compared with homozygous of the major allele (left panels show the miR-608 binding site SNP and the right panels show the miR-125-b binding site SNP).

### DETAILED DESCRIPTION OF THE INVENTION

MicroRNAs (miRs) regulate entire pathways by repressing multiple targets [3], but the biological significance of keeping these numerous interactions balanced is not fully understood. The present invention shows that a single change of a miR-target interaction controlling cholinergic signaling can simultaneously affect multiple other targets and modify physiological phenotypes more profoundly than other SNP interrupting other miR/AChE binding sites. The present inventors show that the primate-specific miR-608 targets acetylcholinesterase (AChE), and that a common single nucleotide polymorphism (SNP rs17228616), in the AChE 3′-untranslated region (3′-UTR) weakens this interaction. Surface Plasmon Resonance measurements demonstrated that miR-608 RNA oligonucleotides bind the major AChE allele (C) more tightly than another known miR-608 target, the Rho GTPase CDC42. In contrast, oligonucleotides carrying the minor AChE allele (A) showed drastically reduced binding of miR-608, far below the strength of miR-608/CDC42 interaction, suggesting shifted target preference in the presence of this allele. Supporting this prediction, the weakened miR-608/AChE interaction resulted in excessive suppression of other targets in cultured human cells and post-mortem brain tissues. Specifically, cortical homogenates from volunteers homozygote for the minor allele showed elevated AChE activity and reduced levels of CDC42 and interleukin-6 (IL6), another miR-608 target. Intriguingly, the increase in AChE and decrease in CDC42/IL6 could synergize each other's effect on neuroimmune activities. This prediction was tested in the HERITAGE Family Study cohort of young healthy volunteers [8]. Subjects heterozygotes and homozygotes for the minor allele showed reduced circulation cortisol levels and elevated systolic and diastolic blood pressure, predicting risk of anxiety and hypertension-related diseases. Taken together, the present invention presents a novel mechanism in which a genetic variant in the AChE binding site interferes with miR-608/AChE interaction, leading to altered target preference, excessive suppression of other targets, and modified medically important parasympathetic-influenced phenotypes. These findings also suggest that other inherited, acquired and therapeutic interference with certain miR functions may similarly show synergistic effects on multiple targets.

Thus, according to a first aspect, the invention relates to a method for the diagnosis of a genetic predisposition of a human subject for at least one AChE-signaling associated disorder. According to more specific embodiments the method comprises the steps of: First, in step (a) genotyping in a biological sample of the tested subject at least one SNP in the 3′UTR of the AChE gene. More specifically, such SNP may be at least one of rs17228616, rs17228602, rs17883268, rs17228609, rs17235010, rs17228602, rs17883268, rs17228609, rs17235010, rs147386700, rs11554090, rs139429533, rs145048252, rs151107784, rs7636, rs149813374, rs145811992, rs113533105, rs148991713, rs3028261, rs145683970, rs61729577, rs74418820, rs41281001, rs138349745, rs116131706, rs144995873, rs17880119, rs17228588, rs8286, rs141785210, rs150598944, rs17881163, rs142818130, rs147370685, rs17228581, rs144986926, rs143875983, rs113406515, rs142452543, rs151335006, rs139672629, rs144426398, rs77109413, rs146564868, rs141299237, rs17885778, rs145703834, rs61729575, rs1056867, rs114367422, rs143172740, rs17228574, rs17234982, rs114782198, rs116298479, rs13246682, rs17881553, rs17886728, rs1799806, rs11554090, rs139429533, rs145048252, rs151107784, rs7636, rs149813374, rs14581192, rs113533105, rs148991713, rs3028261, rs145683970, rs61729577, rs74418820, rs41281001, rs138349745, rs116131706, rs144995873, rs17880119, rs17228588, rs8286, rs141785210, rs150598944, rs17881163, rs142818130, rs147370685, rs17228581, rs144986296, rs143875983, rs113406515, rs142452543, rs151335006, rs139672629, rs144426398, rs77109413, rs146564868, rs141299237, rs17885778, rs145703834, rs61729575, rs1056867, rs114367422, rs143172740, rs17228574, rs17234982, rs114782198, rs116298479, rs13246682 and rs17881553. Further information in connection with the above-mentioned SNPs in provided in Tables 1 and 2, disclosed herein after. The next step (b) involves identifying and detecting in the genotyped sample obtained in step (a) at least one SNP that modulates the binding affinity of at least one of miR-608, miR-125b-3p, miR-4319, miR-125a-5p, miR-761, miR-214, miR-298 and miR-4283 to the 3′UTR of the AChE gene in the sample.

It should be noted that detecting or identifying the presence of at least one SNP that modulates the binding affinity of at least one of said miR-608, miR-125b-3p, miR-4319, miR-125a-5p, miR-761, miR-214, miR-298 and miR-4283 to the 3′UTR of the AChE gene indicates that the tested subject belongs to a predetermined population associated with a genetic predisposition for at least one AChE-signaling associated disorder, thereby providing diagnosis of a genetic predisposition of the tested subject for such disorder.

The invention provides a diagnostic method for detecting a genetic predisposition of a human subject for at least one AChE-signaling associated disorder. The term “genetic predisposition” or the term “genetic susceptibility” as herein defined refers to a genetic-based increase in the risk of developing a disease or to a genetic-based tendency to suffer from a particular condition.

This method is based on detecting a specific nucleotide polymorphism in the AChE 3′UTR. The term “polymorphism” as herein defined refers to a location in the sequence of a gene which varies within a population. A polymorphism is comprised of different “alleles”. For example, C2098A AChE indicates that there is a variation between C and A at the nucleic acid base located at position 2098 in the AChE gene. Because the genotype is comprised of two separate alleles, an individual may be either homozygous or heterozygous for a certain polymorphism (e.g. for the above example, an individual may be either CC, CA or AA). Thus a polymorphism may relate, inter alia, to a single nucleotide polymorphism, as illustrated in the example above.

The term “single nucleotide polymorphism” (SNP) as herein defined, refers to a single base change in the DNA sequence. For a base position with sequence alternatives in genomic DNA to be considered as a SNP, the least frequent allele (the “minor allele” as herein described) should have a frequency of 1% or greater. The most frequent allele is referred to as the “major allele”. SNPs are usually bi-allelic, mainly due to the low frequency of single nucleotide substitutions in DNA. As known to a person skilled in the art, the term “SNP” usually refers to the least frequent allele (i.e. the minor allele), when present in the genome either on both chromosomes (then an individual is said to be homozygous for a certain polymorphism) or on a single chromosome (then an individual is said to be heterozygous for a certain polymorphism).

Known specific SNPs are assigned with unique identifiers, usually referred to by accession numbers with a prefix such as “SNP”, “refSNP” or “rs”, as known to one of skill in the art. Single nucleotide polymorphism database (dbSNP) of nucleotide sequence variation is available on the NCBI website.

In a first step, the method of the invention involves genotyping nucleic acids obtained from the sample. The term “genotyping” as herein defined refers to the identification of the nucleic acid sequence at specific loci in the DNA of an individual. More particularly, the term genotyping refers to the identification of a specific SNP at defined locations in the AChE gene in an individual. Analysis or identification of the particular SNP (i.e. genotyping) in a sample obtained from an individual may be performed by any method well known to one of skill in the art, for example as described in Landegren, U. et al. [9] or in Shi, M. M. [10]. Generally, all current methods involve obtaining nucleic acids from the sample, target sequence amplification, followed by distinction of DNA sequence variants by sequencing or by short hybridization probes, restriction endonucleases, or any other method known in the art. By way of example, genotyping methods may be gel-based genotyping assays. Few exemplary gel-based genotyping assays are provided below.

### PCR-Restriction Fragment Length Polymorphism Analysis

Commonly used methods include gel electrophoresis based techniques such as PCR coupled with restriction fragment length polymorphism analysis. Specific regions of DNA sequences can be PCR amplified. The PCR products then are digested with appropriate restriction enzymes and visualized by staining the gel after electrophoresis.

If the genetic polymorphism produces a gain or loss of the restriction site, a different restriction digestion pattern can be recognized.

### Oligonucleotide Ligation Assay Genotyping

The oligonucleotide ligation assay (OLA) relies on hybridization with specific oligonucleotide probes that can effectively discriminate between the wild-type and variant sequences. Three oligonucleotides are used in OLA: two allele-specific oligonucleotide probes (one specific for the more frequent (major) allele and the other specific for the minor allele) plus a fluorescent common probe. The 3′ ends of the allele-specific probes are immediately adjacent to the 5′ end of the common probe. The gene fragment containing the polymorphic site is amplified by PCR and incubated with the probes. In the presence of thermally stable DNA ligase, ligation of the fluorescent-labeled probe to the allele-specific probe(s) occurs only when there is a perfect match between the variant (minor allele) or the major allele probe and the PCR product template. These ligation products are then separated by electrophoresis, which permits the recognition of the major allele genotypes, the variants (minor allele), the heterozygotes, and the un-ligated probes). By varying the combinations of color dyes and probe lengths, multiple SNPs can be detected in a single reaction.

### Mini-Sequencing

Similar to regular DNA sequencing, mini-sequencing is an efficient way to detect SNPs through the addition of specific nucleotides to a single primer. Several SNP markers can be analyzed in parallel by the use of locus-specific primers and analyzing the allele-specific incorporation of labeled nucleotides.

Genotyping methods may also be based on non-gel-based high-throughput genotyping technologies, particularly useful for large-scale studies. For example, non-gel-based high-throughput genotyping technologies may be based on fluorescence resonance energy transfer detection. Fluorescence resonance energy transfer (FRET) occurs when two fluorescent dyes are in close proximity to one another and the emission spectrum of one fluorophore overlaps the excitation spectrum of the other fluorophore. Commonly used FRET-based technologies include, inter alia, the TaqMan assay and molecular beacons (described herein below).

### TaqMan Genotyping

The TaqMan Allelic Discrimination assay uses the 5′ nuclease activity of Taq polymerase to detect a fluorescent reporter signal generated during or after PCR reactions. For SNP genotyping, one pair of TaqMan probes and one pair of PCR primers are used. The assay uses two TaqMan probes that differ at the polymorphic site, with one probe complementary to the major allele and the other to the variant (minor) allele. A 5′ reporter dye and a 3′ quencher dye are covalently linked to the major or variant (minor) allele probes, where different 5′ reporter dyes are used for each of the major and minor alleles and the 3′ quencher dye is common to both.

When the probes are intact, fluorescence is quenched because of the physical proximity of the reporter and quencher dyes. During the PCR annealing step, the TaqMan probes hybridize to the targeted polymorphic site. In cases where annealing occurred, during the PCR extension phase, the 5′ reporter dye is cleaved by the 5′ nuclease activity of the Taq polymerase, leading to an increase in the characteristic fluorescence of the reporter dye. Specific genotyping is determined by measuring the signal intensity of the two different reporter dyes after the PCR reaction.

In some embodiment, analysis of SNPs in a sample obtained from an individual may be performed by TaqMan genotyping, as described in the Examples section below.

### Molecular Beacons

Molecular beacons are oligonucleotide probes that have two complementary DNA sequences flanking the target DNA sequence and a donor-acceptor dye pair at opposite ends of each probe. When not hybridized to the target, the probe adopts a hairpin-loop conformation with the reporter and quencher dyes close together, and therefore, no donor fluorescence is generated. When hybridized to the right target sequence, the two dyes are separated and the fluorescence is increased dramatically. Mismatched probe-target hybrids dissociate at substantially lower temperature than exactly complementary hybrids. This thermal instability of mismatched hybrids increases the specificity of molecular beacons. For SNP genotyping, two molecular beacons with exact sequence matches to the major and variant (minor) alleles are used in the same PCR reaction. These two molecular beacons are labeled with different fluorophores that emit fluorescent light at distinct optical wavelengths. The use of two differentially labeled molecular beacons in the same PCR reaction allows simultaneous determination of three possible allelic combinations.

In some embodiments analysis of SNPs in a sample obtained from an individual may be performed by allele-specific ligation. By way of example, allele-specific ligation-based technologies may be Dye-labeled oligonucleotide ligation or rolling circle amplification (RCA).

### Dye-Labeled Oligonucleotide Ligation

Dye-labeled oligonucleotide ligation combines PCR-OLA with FRET detection in a one-step homogeneous assay. The PCR primers used in the assay are designed to have high melting temperatures. The three dye-labeled ligation probes for each SNP are designed to have low melting temperatures. A 5′ donor dye-labeled common probe terminates one base immediately upstream from the polymorphic site. Two allele-specific 5′-phosphorylated, 3′-acceptor dye-labeled probes have polymorphic nucleotides at the 5′ end. A thermostable DNA polymerase with no 5′ nuclease activity (AmpliTaq FS) and a thermostable DNA ligase are used. The first stage of PCR reaction is kept at high temperature, and the ligation probes are unable to anneal. After sufficient PCR products are generated, the second stage of the reaction, with a low annealing temperature, allows ligation to occur. By analyzing the fluorescence signals of all the dyes, individual genotypes can be determined directly after one reaction using real-time PCR or by end-point signal analysis using a fluorescent plate reader.

### Rolling Circle Amplification (RCA)

According to the RCA method, which is sufficiently sensitive to be applied to genomic DNA, for each SNP, two allele-specific probes are designed to discriminate between the two alleles. Each probe consists of a single oligonucleotide, 80-90 bases in length. The 5′ end of the probe is phosphorylated and bears a sequence of 20 nucleotides that is complementary and, therefore, will hybridize to the region immediately 5′ of the SNP. The 3′ end of the probe contains 10-20 nucleotides complementary to the region immediately 3′ of the SNP. Both allele-specific probes are identical with the exception of the 3′ base, which is varied to complement the two alleles at the polymorphic site. The two probes contain two different generic backbone sequences. A generic backbone that encodes binding sites for two RCA amplification primers is sandwiched between the allele-specific probe arms. The first stage of the assay involves ligation of the probes to the target DNA by allelic discrimination. After denaturation of the target genomic DNA, both allele-specific probes are then added to the denatured DNA. A stable hybrid is formed only if the 3′ base of the probe is perfectly matched to the polymorphic nucleotide present in the target. Complete hybridization aligns the two ends of the probe together on the target DNA and circularizes the probe by a thermostable ligase. Amplification of a circularized probe by RCA requires the first primer to hybridize to its complementary region on the probe backbone. In the presence of a strand-displacing DNA polymerase, the primer is extended, eventually displacing itself at its 5′ end once one complete revolution of the circularized probe is made. Continued polymerization and displacement generates a single-stranded, concatameric DNA copy of the original probe.

In some embodiments analysis of SNPs in a sample obtained from an individual may be performed by allele-specific nucleotide incorporation, for example, by pyrosequencing or by Single-base extension with fluorescence detection as detailed below.

### Pyrosequencing

Pyrosequencing detects de novo incorporation of nucleotides based on the specific template. The incorporation process releases a pyrophosphate, which is converted to ATP in the presence of adenosine 5′-phosphosulfate, which in turn stimulates luciferase. The light production in the luciferase-catalyzed reaction is detected by a charge couple device camera. The height of each peak correlates to the light signal and is proportional to the number of nucleotides incorporated. ATP and unincorporated dNTPs are continuously degraded by apyrase. The light is switched off, and the next dNTP is added. As the process continues, the complementary DNA strand is built up and the nucleotide sequence is determined from the signal peak in the pyrogram.

Single-Base Extension with Fluorescence Detection

One of the commonly used SNP genotyping methods is the template-directed dye terminator incorporation assay with fluorescent polarization (FP) detection. FP is based on the principle that a small molecule tumbles rapidly in solution. If plane-polarized light is shone on fluorescent dye labels, the molecules tumble rapidly, and the emission is depolarized. If the viscosity and temperature are constant, FP is directly proportional to the molecular volume, which is directly proportional to the molecular weight. The sequencing primer is immediately upstream from the polymorphic site. When incubated in the presence of dideoxynucleotide triphosphates labeled with different fluorophores, the allele-specific dye-labeled dideoxynucleotide triphosphate is linked to the primer in the presence of DNA polymerase and target DNA. The genotype of the target DNA molecule can be determined simply by exciting the fluorescent dye in the reaction and determining the change in FP.

In some other embodiments analysis of SNPs in a sample obtained from an individual may be performed by DNA microarray genotyping. The DNA microarray is a hybridization-based genotyping technique that offers simultaneous analysis of many polymorphisms. High-density microarrays are created by attaching hundreds of thousands of oligonucleotides to a solid silicon surface in an ordered array. The DNA sample of interest is PCR amplified to incorporate fluorescently labeled nucleotides and then hybridized to the array. Each oligonucleotide in the high-density array acts as an allele-specific probe. Perfectly matched sequences hybridize more efficiently to their corresponding oligomers on the array and, therefore, give stronger fluorescent signals over mismatched probe-target combinations. The hybridization signals are quantified by high-resolution fluorescent scanning and analyzed by computer software. DNA alterations such as heterozygous base-pair polymorphisms or mutations, insertions, and deletions can be identified.

It should be noted that genotyping according to the invention may be performed by any technique known in the art and specifically, any of the procedures described herein or any combinations thereof.

The method of the invention is based on the detection of SNPs in the AChE 3′ UTR. The term “AChE” as used herein encompasses both the gene coding acetylcholinesterase (AChE, for example, human AChE having the accession number NG_{—}007474.1 and as denoted SEQ ID NO. 19), the RNA transcripts encoded by the AChE gene (i.e., alternatively spliced RNA molecules) and the various isoforms of the AChE protein (e.g. AChE-S, having the accession number NM_{—}000665.3 and also denoted SEQ ID NO. 20 and AChE-R, having the accession number NM_{—}015831.2 and also denoted SEQ ID NO. 21).

Acetylcholinesterase (AChE) catalyses the rapid hydrolysis of acetylcholine (ACh) to acetate and choline. The principal biological role of AChE, which is mechanistically defined as a serine hydrolase, is termination of impulse transmission at cholinergic synapses. In addition, AChE is implicated in nerve and muscle development, in hematopoiesis as well as in different pathologies, to name but few, Alzheimer disease (AD), Gulf War syndrome, and hypersensitivity to pesticides.

Alternative splicing allows the production of three distinct AChE variants (or isoforms), each with a different carboxy-terminal (C-terminal) sequence, namely, the ‘synaptic’ (S), ‘erythrocytic’ (E) and ‘readthrough’ (R) AChE isoforms.

In AChE-S, a cysteine located three residues from the carboxyl terminus of the human protein allows dimerization by disulphide bridging. Two additional monomers can become associated by hydrophobic interactions. These tetramers can attach covalently to a hydrophobic P subunit or to a collagen-like protein known as the T subunit. In AChE-E, a glycyl bond near the carboxyl terminus undergoes transamidation to attach a glycophosphatidylinositol group to the protein, which anchors the mature AChE-E to the outer surface of erythrocytes. It is noteworthy that another nomenclature labels the synaptic and erythropoietic variants according to properties of the proteins, i.e. they are termed T (tailed), and H (hydrophobic), respectively. AChE has also various biological functions which are not necessarily dependent on its hydrolytic capacity. For example, it has been reported that AChE is involved in neuritogenesis, cell adhesion and activation of dopamine neurons. AChE was also reported to promote amyloid fiber assembly and to be involved in inflammation, haematopoiesis and thrombopoiesis.

As indicated above, the diagnostic method of the invention is based on detecting specific SNPs at the AChE 3′UTR that modulate the binding of specific miRs to their target sites, and thereby modulate their regulatory activity. The term “three prime untranslated region” (3′-UTR) as herein defined refers to the section of messenger RNA (mRNA) that immediately follows the translation termination codon of a gene. An mRNA molecule is transcribed from the DNA sequence and is later translated into protein. Several regions that comprise the mRNA molecule are not translated into protein, including the 5′ cap, 5′ untranslated region, 3′ untranslated region, and the poly(A) tail. The 3′-UTR often contains several regulatory regions affecting post-transcriptional gene expression. “MicroRNAs” (“miRNAs” or “miRs”) as used herein are post-transcriptional regulators that bind to complementary sequences in the three prime un-translated regions (3′ UTRs) of target messenger RNA transcripts (mRNAs), usually resulting in gene silencing. miRNAs are short ribonucleic acid (RNA) molecules, on average only 22 nucleotides long. The human genome may encode over 1800 miRNAs, which may target over 60% of mammalian genes and are abundant in many human cell types. Each miRNA may repress hundreds of mRNAs. miRNAs may be well conserved in eukaryotic organisms or primate-specific, and are thought to be a vital and evolutionarily ancient or younger components of genetic regulation. miRNA genes are usually transcribed by RNA polymerase II (Pol II). The polymerase often binds to a promoter found near the DNA sequence encoding what will become the hairpin loop of the pre-miRNA. The resulting transcript is capped with a specially-modified nucleotide at the 5′ end, polyadenylated with multiple adenosines (a poly(A) tail), and spliced. The product, called a primary miRNA (pri-miRNA), may be hundreds or thousands of nucleotides in length and contain one or more miRNA stem loops. In other cases, miRNAs are transcribed as an integral parts of a host gene, often within an intron of such genes, and are cut off the intronic sequence during splicing. The resultant hairpin loop structures are further processed by enzymatic nuclear proteins, specifically, DGCR8 (or “Pasha” in invertebrates) and Drosha. The resulting hairpin, known as a pre-miRNA, has a two-nucleotide overhang at its 3′ end; it has 3′ hydroxyl and 5′ phosphate groups.

Pre-miRNA hairpins are exported from the nucleus and in the cytoplasm, the pre-miRNA hairpin is cleaved by the RNase III enzyme Dicer. This endoribonuclease interacts with the 3′ end of the hairpin and cuts away the loop joining the 3′ and 5′ arms, yielding an imperfect miRNA:miRNA* duplex about 22 nucleotides in length. Over all hairpin length and loop size influence the efficiency of Dicer processing, and the imperfect nature of the miRNA:miRNA* pairing also affects cleavage. Although either strand of the duplex may potentially act as a functional miRNA, only one strand is usually incorporated into the RNA-induced silencing complex (RISC) where the miRNA and its mRNA target interact.

The mature miRNA is part of an active RNA-induced silencing complex (RISC) containing Dicer and many associated proteins. RISC is also known as a microRNA ribonucleoprotein complex (miRNP); RISC with incorporated miRNA is sometimes referred to as “miRISC.”

The prefix “mir” is followed by a dash and a number, the latter often indicating order of naming. For example, mir-123 was named and likely discovered prior to mir-456. The uncapitalized “mir-” refers to the pre-miRNA, while a capitalized “miR-” refers to the mature form. miRNAs with nearly identical sequences by one or two nucleotides are annotated with an additional lower case letter. For example, miR-123a would be closely related to miR-123b. miRNAs that are 100% identical but are encoded at different places in the genome are indicated with additional dash-number suffix: miR-123-1 and miR-123-2 are identical but are produced from different pre-miRNAs. Species of origin is designated with a three-letter prefix, e.g., hsa-miR-123 would be from human (Homo sapiens). microRNAs originating from the 3′ or 5′ end of a pre-miRNA are denoted with a -3p or -5p suffix. When relative expression levels are known, an asterisk following the name indicates an miRNA expressed at low levels relative to the miRNA in the opposite arm of a hairpin. For example, miR-123 and miR-123* would share a pre-miRNA hairpin, but relatively more miR-123 would be found in the cell.

Still further, the method of the invention concerns detecting SNPs that modulate binding of certain miRNAs to the 3′UTR of the AChE gene. As herein defined, the term “binding affinity” refers to the strength of interaction between two species. The forces controlling the affinity are thermodynamic. For example, binding affinity may be measured for the interaction between a drug and the receptor thereof or as in the present case, for the interaction between a specific miRNA and its target on a mRNA molecule. Such modulated binding may change the function of the miRNAs. As used herein, the phrase “function of the miRNA” relates to binding, attaching, regulating, processing, interfering, augmenting, stabilizing and/or destabilizing a miRNA target, i.e., the target that is regulated by the action and/or presence of the micro-RNA. It should be appreciated that the term modulation refers either to elevation or reduction of the binding of the miRNA to its specific binding site. Thus, in some embodiments, the specific SNP may inhibit and reduce the binding affinity of a specific miRNA to AChE 3′UTR, thereby reducing suppression of said miRNA that results in an increase of the expression and activity of AChE. Non-limiting examples for such inhibitory SNPs are rs17228616 and rs17228602 that reduce binding of miR-608 and miR-125b to AChE 3′UTR. More specifically, “inhibition”, “moderation”, “reduction” or “attenuation” as referred to herein, relate to the retardation, restraining or reduction of such binding and thereby the regulatory function of the specific miRNA, by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, as compared to the binding or function of said miRNA to the site in the major allele. In other embodiments, the specific SNP may enhance and therefore increase the binding affinity of a specific miRNA to AChE 3′UTR, thereby enhancing suppression of said miRNA that results in a decrease of the expression and activity of AChE. The terms “increase”, “elevation”, “enhancement” or “potentiation” as referred to herein, relate to the enhancement and increase of such binding and thereby the regulatory function of the specific miRNA, by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

According to some embodiments, the diagnostic method of the invention comprises the steps of:

First in step (a) genotyping in a biological sample of the tested subject at least one SNP in the 3′UTR of the AChE gene, said SNP is at least one of rs17228616, rs17228602, rs17883268 and rs17235010. In the second step (b) determining, identifying and detecting the presence or the absence of at least one of C2098A of SNP rs17228616, G2165A of SNP rs17228602, T2402C of SNP rs17883268 and G2071A of SNP rs17235010 in at least one allele of the AChE gene in said sample. The presence of at least one of A at rs17228616, A at rs17228602, C at rs17883268 and A at rs17235010 in at least one allele of the AChE gene indicates that the tested subject belongs to a predetermined population associated with a genetic predisposition for at least one AChE-signaling associated disorder.

According to one embodiment, the presence of at least one of A at rs17228616 (more specifically, adenine at position 2098 of SEQ ID NO. 20), A at rs17228602 (specifically, adenine at position 2165 of SEQ ID NO. 21), C at rs17883268 (specifically, cytosine at position 2402 of SEQ ID NO. 21) and A at rs17235010 (specifically, adenine at position 2071 of SEQ ID NO. 20), in at least one allele of the AChE gene (e.g. as exemplified above for AChE-S having the SEQ ID NO. 20 or for AChE-R having the SEQ ID NO. 21), leads to modulation of the binding affinity of at least one of miR-608, miR-125b-3p, miR-4319, miR-125a-5p, miR-4283, miR-761, miR-214 and miR-298 to the 3′UTR of AChE gene thereby leading to a modulation of at least one of the level and catalytic activity of AChE and the expression level of at least one gene target of at least one of miR-608, miR-125b-3p, miR-4319, miR-125a-5p, miR-761, miR-214, miR-298 and miR-4283. As noted above, such modulation may result in either enhancement or inhibition of the binding of a specific miRNA to AChE 3′UTR. Thus, in specific embodiments, reduction or decrease in the binding affinity of such miRNA may result in a reduced suppression of the expression and the activity of AChE by said miRNA and thereby to elevation thereof. This may shift the specific miRNA to other gene targets, enhance its suppressive effect, thereby leading to reduction in the expression of these other targets. In yet other alternative embodiments, the specific SNP may enhance the binding affinity of a specific miRNA to the AChE 3′UTR, thereby increase suppression of the expression and the activity of AChE by said miRNA leading to reduction thereof. This may result in decreased suppression of other targets of said miRNA and thereby elevated expression of these other targets.

It should be understood that “A” refers to adenine, “T” refers to thymine, “C” relates to cytosine and “G” refers to guanine.

The method of the invention relates to diagnostic methods for determining genetic predisposition of a subject to an AChE-associated disorder. As noted above, these methods are based on detecting specific SNPs that modulate, either disturb or alternatively potentiate the binding of specific miRNAs to the 3′UTR of AChE, and thereby impair the regulation of different targets of such miRNAs. Specific embodiments of the invention relate to the following miRNAs, miR-608, miR-125b-3p, miR-4319, miR-125a-5p, miR-761, miR-214, miR-298 and miR-4283.

Homo sapiens microRNA 608 (also denoted miR-608) as herein defined is of the nucleic acid sequence as denoted by SEQ ID NO. 3 (the mature miR-608 having the nucleic acid sequence of 5′-AGGGGUGGUGUUGGGACAGCUCCGU-3′). miR-608 was reported to be primate-specific and to bind its target within the insulin receptor (INSR), with additional (predicted or validated) targets including interleukin-6 (IL-6), CDC42, CDC44, interleukin-1 alpha (IL1A), growth hormone receptor (GHR), and TP53. miR-608 was also reported to be associated with various cancers, for example colorectal cancer and breast cancer. Complete nucleic acid sequence of hs-miR-608 (NR_{—}030339): GGGCCAAGGTGGGCCAGGGGTGGTGTTGGGACAGCTCCGTTTAAAAAGGCATCT CCAAGAGCTTCCATCA AAGGCTGCCTCTTGGTGCAGCACAGGTAGA, as also denoted by SEQ ID NO. 50.

Homo sapiens microRNA 125b-3p (also denoted miR-125b-3p or as previously denoted hsa-miR-125b) as herein defined is of the nucleic acid sequence as denoted by SEQ ID NO. 31 (the mature miR-125b-3p having the nucleic acid sequence of 5′-UCCCUGAGACCCUAACUUGUGA-3′). Among the various targets of this miRNA are the p53, MUC1 and TNF-α homo sapiens genes to name but few. Complete nucleic acid sequence of hs-miR-125b-3p (MI0000470): ACCAGACUUUUCCUAGUCCCUGAGACCCUAACUUGUGAGGUAUUUUAGUAACA UCACAAGUCAGGCUCUUGGGACCUAGGCGGAGGGGA, as also denoted by SEQ ID NO. 30.

Homo sapiens microRNA 4319 (also denoted miR-4319) as herein defined is of the nucleic acid sequence as denoted by SEQ ID NO. 23 (the mature miR-4319 having the mature nucleic acid sequence of 5′-UCCCUGAGCAAAGCCAC-3′). miR-4319 was reported in connection with the molecular pathogenesis underlying the primary myelofibrosis (PMF) progression to acute myeloid leukemia (AML). A study conducted by Albano F. et al. [11] reported a PMF case evolved to AML showing the down-regulation of the intronic miR-4319 and the over-expression of its host gene, SET binding protein (SETBP1). This study thus suggests a possible molecular mechanism regulating the PMF progression to AML which involves miR-4319. Complete nucleic acid sequence of hs-miR-4319 (NR_{—}036203): TTGGCTTGAGTCCCTGAGCAAAGCCACTGGGAATGCTCCCTGAGGACGTTATATG AGTGCTCAGCTCATGGGGCTATGATGGTCA, as also denoted by SEQ ID NO. 22.

Homo sapiens microRNA 125a-5p (also denoted miR-125a-5p or as previously denoted miR-125a) is of the nucleic acid sequence as denoted by SEQ ID NO. 27 (the mature miR-125a-5p having the nucleic acid sequence of 5′-UCCCUGAGACCCUUUAACCUGUGA-3′). miR-125a-5p was reported to be associated with human cervical carcinoma. Among the various targets of this miRNA are LIN28A, NTRK3 and CD34 to name but few.

Complete Nucleic acid sequence of hsa-mir-125a-5p (MI0000469): UGCCAGUCUCUAGGUCCCUGAGACCCUUUAACCUGUGAGGACAUCCAGGGUCA CAGGUGAGGUUCUUGGGAGCCUGGCGUCUGGCC, as also denoted by SEQ ID NO. 26.

Homo sapiens microRNA 761 (also denoted miR-761) as herein defined is of the nucleic acid sequence as denoted by SEQ ID NO. 39 (the mature miR-761 having the nucleic acid sequence of 5′-GCAGCAGGGTGAAACTGACACA-3′). miR-761 was reported to bind to various genes, inter alia, APOBEC2, and CCDC21.

Complete Nucleic acid sequence of hs-miR-761 (NR_{—}031580): GGAGGAGCAGCAGGGTGAAACTGACACAGTTCTGGTGAGTTTCACTTTGCTGCTC CTCC, as also denoted by SEQ ID NO. 38.

Homo sapiens microRNA 214 (also denoted miR-214) as herein defined is of the nucleic acid sequence as denoted by SEQ ID NO. 43 (the mature miR-214 having the nucleic acid sequence of 5′-ACAGCAGGCACAGACAGGCAGU-3′). miR-214 is a “melano-miR”, since it is associated with metastasis of melanoma. Mature microRNA excised from miR-214 is predicted to target two activating protein 2 transcription factors, resulting in downstream effects on a number of genes regulating vital cell cycle processes, such as apoptosis, proliferation and angiogenesis. miR-214 has been found to be down-regulated in human cervical cancer; when miR-214 was up-regulated in HeLa cancer cells, it was found to significantly reduce cell growth. The increased expression of miR-214 in pancreatic cancer could bring about increased resistance to chemotherapy. Complete nucleic acid sequence of hs-miR-214 (MI0000290): GGCCUGGCUGGACAGAGUUGUCAUGUGUCUGCCUGUCUACACUUGCUGUGCAG AACAUCCGCUCACCUGUACAGCAGGCACAGACAGGCAGUCACAUGACAACCCA GCCU, as also denoted by SEQ ID NO. 42.

Homo sapiens microRNA 298 (also denoted miR-298) as herein defined is of the nucleic acid sequence as denoted by SEQ ID NO. 47 (the mature miR-298 having the nucleic acid sequence of 5′-AGCAGAAGCAGGGAGGUUCUCCCA-3′). miR-298 was demonstrated to recognize specific binding sites in the 3′-UTR of BACE1 mRNA and exert regulatory effects on BACE1 protein expression in cultured neuronal cells, which may provide the molecular basis underlying BACE1 deregulation in Alzheimer's disease [12]. It was also shown that miR-298 is a part of the imprinted Gnas/GNAS clusters in both mice and humans [13]. Complete nucleic acid sequence of hs-miR-298 (MI0005523): UCAGGUCUUCAGCAGAAGCAGGGAGGUUCUCCCAGUGGUUUUCCUUGACUGU GAGGAACUAGCCUGCUGCUUUGCUCAGGAGUGAGCU, as also denoted by SEQ ID NO. 46.

Homo sapiens microRNA 4283 (also denoted miR-4283 or hsa-mir-4283-1) as herein defined is of the nucleic acid sequence as denoted by SEQ ID NO. 35 (the mature miR-4283 having the nucleic acid sequence of 5′-UGGGGCUCAGCGAGUUU-3′). Among the various targets of this miR are SIGLEC8, GNG13, and SENP1 to name but few.

Complete nucleic acid sequence of hs-miR-4283 (MI0015892): ACUCUGAUCCUGGGGCUCAGCGAGUUUGCAAGGGGUGUUUCUGUCCAUGGUC AGGCUUGCCAGCCUUGGUCCUUGGGCCC, as also denoted by SEQ ID NO. 34.

Some specific embodiments of the invention may be based on the detection of a specific SNP that disturbs the binding and thereby the function of miR-608. Such method of the invention comprises the steps of: First step (a), involves genotyping in a biological sample of the examined subject SNP rs17228616. The second step (b) concerns determining or identifying the presence or the absence of C2098A of SNP rs17228616 in at least one allele of the AChE gene in the tested genotyped sample obtained in step (a). The presence of at least one A allele at rs17228616, or more specifically, the presence of adenine in position 2098 of rs17228616 in at least one allele of the AChE gene, indicates that the tested subject belongs to a predetermined population associated with a genetic predisposition for at least one AChE-signaling associated disorder.

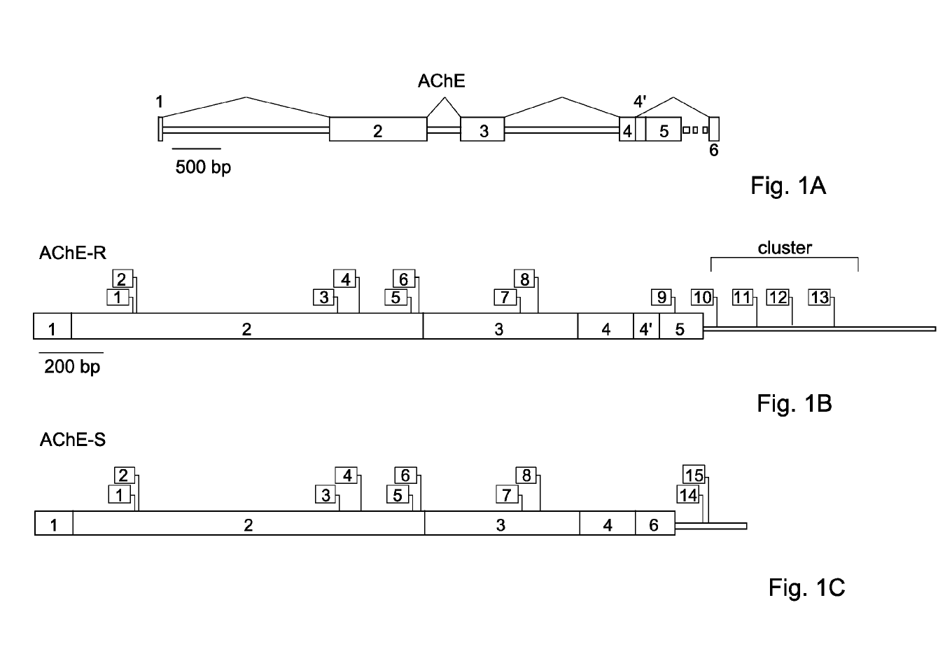

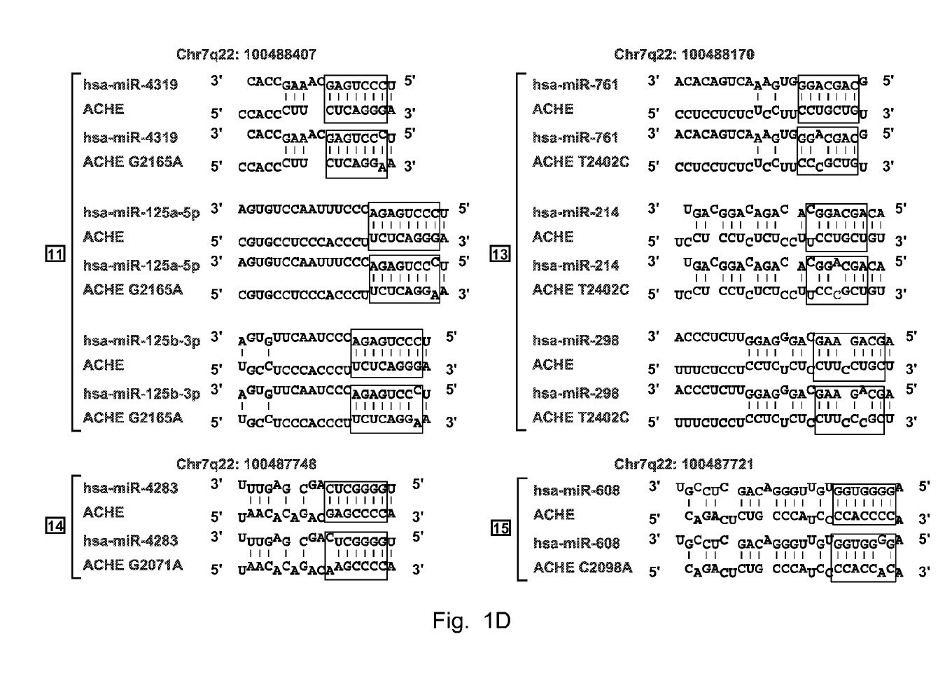

As indicated in FIG. 1, the particular binding site of miR-608 on the 3′UTR of AChE, is primate specific. Therefore, in certain particular and non-limiting embodiments, rodent animal models may not be applicable in this case.

As shown in Example 3, the presence of at least one A allele at rs17228616 in a genotyped sample of a tested subject is associated with reduction of the binding affinity of miR-608 to the 3′UTR of AChE gene thereby resulting in at least one of an increase in the level and catalytic activity of AChE and decrease in the expression level of at least one other gene target of miR-608. As noted above, gene target of miR-608 include interleukin-6 (IL-6), CDC42, CDC44, interleukin-1 alpha (IL1A), growth hormone receptor (GHR), and TP53. In more specific embodiments, target of miR-608 is at least one of the Rho GTPase CDC42 and Interleukine-6 (IL-6).

Interleukin 6 (IL-6, also known as BSF2, HGF, HSF, IFNB2, Gene ID: 3569) is a single-chain protein that is produced by T cells, B cells, monocytes, fibroblasts and certain other cell types. This cytokine is a multifunctional cytokine that was originally identified as a B-cell differentiation factor involved in the maturation of antibody-producing cells. Since then, IL-6 has been found to have a wide array of additional activities, including effects on T cells, blood vessels, and neurons.

IL-6 exerts a significant influence on the course of inflammation in humans. There is evidence that IL-6 is capable of mediating both pro-inflammatory effects, including the induction of intercellular adhesion molecules and the recruitment of leukocytes, and anti-inflammatory effects, such as suppression of the pro-inflammatory cytokines, tumor necrosis factor and IL-1. The balance between the pro-inflammatory and anti-inflammatory effects of IL-6 may influence the development of chronic inflammation and disease, for example, systemic juvenile arthritis, systemic lupus erythematosus, Crohn's disease, and rheumatoid arthritis (RA).

In certain embodiments, IL-6 as referred to herein, is the human IL-6, as denoted by Accession number of (M18403), the amino acid sequence as denoted by SEQ ID NO. 55, encoded by the nucleic acid sequence of SEQ ID NO. 56.

In yet another embodiment, a target of miR-608 may be CDC42. Cell Division Cycle 42 (CDC42 or Cdc42, also known as G25K, CDC42Hs, Gene ID: 998) encodes a 21.3 kDa, 191 amino acids small GTPase protein that belongs to the Rho family of Ras GTPases superfamily. CDC42 is highly conserved, and has been found to be a molecular switch that modulates a variety of cellular processes in organisms from yeast to mammals, including regulation of actin cytoskeletal architecture and cell polarity. Several studies have implicated Cdc42 in endocytosis, ER/Golgi interface vesicle trafficking, post-Golgi transport and exocytosis.

CDC42 plays a role in a wide variety of cellular processes that are dependent on the actin cytoskeleton, such as cytokinesis, phagocytosis, cell migration, morphogenesis, chemotaxis and axon guidance. Physiologically, CDC42 is implicated in other essential cellular processes such as axon myelination, intracellular trafficking, gene transcription, cell-cycle regulation and cell fate determination. Deregulation of CDC42 is found in several pathogenic processes such as cancer, neurodegenerative disorders and cardiovascular disease (Based on, inter alia, Xiaojuan C. et al. [15]) In certain embodiments, CDC42 as referred to herein may be the human CDC42, as denoted by Accession number of (BC018266), the amino acid sequence as denoted by SEQ ID NO. 57, encoded by the nucleic acid sequence of SEQ ID NO. 58. Although the embodiments refer to the human CDC42, the invention may further relate to the mouse (Mus musculus) CDC42 as denoted by GeneBank accession number 12540 (Gene ID).

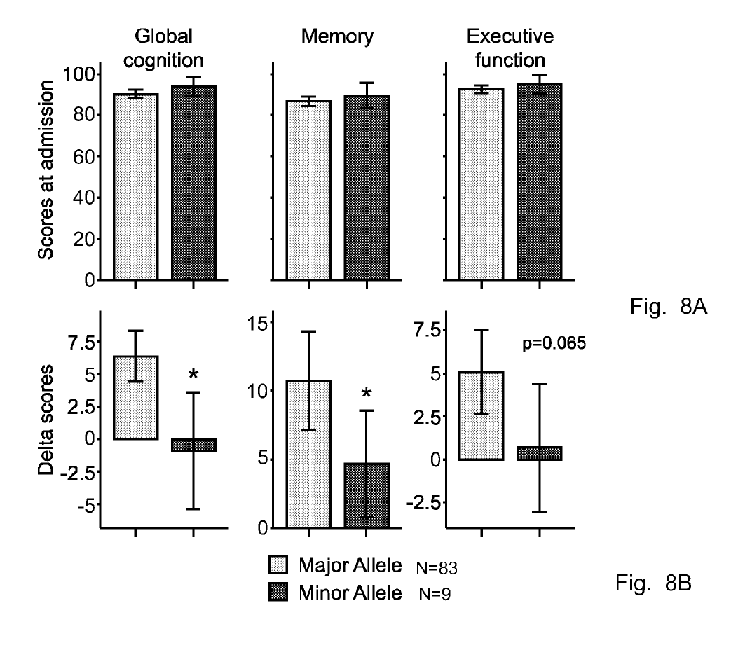

As shown in FIG. 8, and Example 6, the specific SNP rs17228616 has been found by the present invention in correlation with poor or mal-recovery of patients from an AChE-associated disorder, specifically, ischemic disorder, more specifically, an ischemic stroke. Therefore, the method of the invention may provide a powerful tool of specific diagnosis (personalized medicine) and selection of such particular population of patients and thereby provides an early and appropriate treatment that may combine for example, standard therapeutic agents used for treatment of such specific disorder with the miR-608 and AChE antagonists described herein after. Thus, the invention further provides the diagnosis and prognosis of mal-recovery from at least one AChE-signaling associated disorder. In certain embodiments, such method comprises the steps of genotyping in a biological sample of said subject SNP rs17228616. It should be noted that identifying the presence of at least one A allele at rs17228616 indicates that the subject belongs to a predetermined population associated with a mal-recovery from said AChE-signaling associated disorder. In more specific embodiments, such disorder may be for example, acute ischemic stroke. Still further, in this connection it is interesting to note that Interleukin-6 is a major regulator of muscle waisting under septic shock. Therefore a particular SNP that modulates the levels of IL-6 may worsen the prospects of carriers to survive such events. In certain embodiments, the diagnostic methods of the invention may thus assist in identification of patients having such poor prognosis and in determination of proper therapeutic regimen reversing this aberrant modulation. It should be further noted that this may be also specifically relevant to any of the AChE-associated muscle diseases described herein.

As herein defined, the term “mal recovery” refers to a prolonged act, process, duration, or an instance of recovering, to an absence of such process, or to a reduced survival of a subject carrying the minor allele in a certain miRNA binding site with respect to a subject carrying the major allele thereof.

As shown in Example 7, SNP rs17228602 disturbs the binding of miR-125b-3p to the AChE 3′UTR. Thus, some specific embodiments, the diagnostic methods of the invention may be based on a specific SNP that disturbs the binding and thereby the regulatory function of miR-125-b. According to such embodiments the following steps may be applied: In a first step (a) genotyping in a biological sample of said subject SNP rs17228602. The second step (b), involves determining and/or identifying the presence or the absence of G2165A of SNP rs17228602 in at least one allele of the AChE gene in the genotyped sample of step (a). It should be noted that the presence of A allele at rs17228602 indicates that the examined subject belongs to a predetermined population associated with a genetic predisposition for at least one AChE-signaling associated disorder. According to certain embodiments, the presence of A allele at rs17228602 in the examined subject leads to reduction of the binding affinity of miR-125b-3p to the 3′UTR of AChE thereby leading to at least one of an increase in the level and catalytic activity of AChE and decrease in the expression level of at least one gene target of miR-125b-3p.

As noted above, the diagnostic methods of the invention provide information regarding the detection of specific population of patients that may benefit from a treatment regimen directed at reversing the disturbed modulation of different target genes by their specific miRNAs, as a result of existence of SNPs. Thus, some embodiments disclosed herein concern methods combining diagnostic steps as described by the invention, that facilitate selection of the appropriate patients population, with a therapeutic step. According to such embodiments, the methods of the invention may further comprise the step of administering to a subject displaying the presence of at least one of the SNPs of the invention, namely, A at rs17228616, A at rs17228602, C at rs17883268, A at rs17228609 and A at rs17235010, a therapeutic effective amount of at least one of: (i) at least one antagonist of at least one of miR-608, miR-125b-3p, miR-4319, miR-125a-5p, miR-761, miR-214, miR-298 and miR-4283; and (ii) at least one AChE antagonist.

Antagonists of microRNA molecules (miRNAs) are generally known to a person skilled in the art. Generally, miRNA inhibitors are able to competitively bind specific miRNAs and prevent them from regulating their endogenous targets. There are some available techniques designed to inhibit miRNA function, including the introduction of short, single-stranded chemically modified inhibitor oligonucleotides such as 2′-O-methyl(2′-OMe) RNA, locked nucleic acid (LNA) molecules and ‘antagomirs’. These reagents are chemically synthesized to have complementarity to mature miRNAs and are generally resistant to cellular nucleases. Inhibition of miRNA function may also be effected by transfecting cells with DNA vectors expressing a miRNA inhibitor (e.g. lentivirus-derived vehicles). In this case, the transcripts containing the miRNA targets are designed to accumulate in a cell and act as a decoy or sponge for the cognate miRNA, thus interfering with the ability of the miRNA to regulate its natural targets.

In some embodiments, miR antagonists as herein defined may be antagomirs. Antagomirs are a class of chemically engineered oligonucleotides that specifically silence miRNA expression. Antagomir molecules are based on single-stranded RNA molecules, 21-23 nucleotides in length that are perfectly complementary to the specific miRNA target with either mis-pairing at the cleavage site of Ago2 (a protein which plays a role in RNA interference) or some sort of base modification to inhibit Ago2 cleavage. Usually, antagomirs have some sort of modification (e.g. 2′-methoxy groups and phosphorothioates), rendering them more resistant to degradation. In certain embodiments, the antagomirs of the invention may be phosphorothioated, LNA-modified oligonucleotide [14] complementary to the mature desired miR.

Blockmirs are designed to have a sequence that is complementary to an mRNA sequence that serves as a binding site for microRNA. Upon binding, Blockmirs sterically block microRNA from binding to the same site, which prevents the degradation of the target mRNA via RNA-induced silencing complex (RISC).

In some embodiments, miR antagonists as herein defined may be miRNA sponges, i.e. in vivo expressed transcripts that contain multiple miRNA antisense binding sites (MBS) to sequester miRNAs. Sponge RNAs contain complementary binding sites to a miRNA of interest, and are produced from transgenes within cells. As with most miRNA target genes, a sponge's binding sites are specific to the miRNA seed region, which allows them to block a whole family of related miRNAs. In addition, Sponges with an imperfect MBS, i.e. a MBS that include a 4 nucleotide central bulge (referred to as “bulged sponges”), are also encompassed by the present disclosure.

In further embodiments, miR antagonists as herein defined may be Tough Decoys (also referred to as TuD RNA) containing two miRNA recognition sites, e.g. short, hairpin-shaped RNAs for simultaneous suppression of two or more miRNAs. Tough decoy RNAs are double-stranded, and this feature, along with a stem-loop stabilized secondary structure, resists cellular nuclease degradation and facilitates sustained miRNA inhibition for longer than one month. In addition, both strands of a TuD RNA contain a miRNA binding site for more efficient sequestration of target miRNAs at lower, nanomolar concentrations. These decoys may also be expressed intracellularly from lentivirus-derived gene vectors.

Still further, the therapeutic methods of the invention may involve, in addition to miRNA antagonists, the administration of AChE antagonists or inhibitors.

AChE inhibitors as herein defined is any agent which is capable of blocking or hindering AChE Inhibition of AChE may be effected by lowering the expression of AChE by specifically targeting AChE mRNA molecules or by inhibiting the AChE enzyme.

Thus in some embodiments, an AChE inhibitor according to the invention may be any agent which is capable of blocking or hindering the AChE enzyme from hydrolyzing acetylcholine, thereby increasing both the level and duration of action of the this neurotransmitter. There are reversible, quasi-irreversible (or pseudo-irreversible in some sources) and irreversible AChE inhibitors.

In some embodiments, the acetylcholinesterase inhibitor according to the invention may be a reversible competitive or noncompetitive inhibitor of cholinesterase, e.g. Delta9-tetrahydrocannabinol (THC), Carbamates (Physostigmine, Neostigmine, Pyridostigmine, Ambenonium, Demarcarium and Rivastigmine), Phenanthrene derivatives (e.g. Galantamine), Caffeine, Piperidines (e.g. Donepezil), Tacrine (also known as tetrahydroaminoacridine, Edrophonium, Huperzine A, Ladostigil, Ungeremine and Lactucopicrin. In some other embodiments, the acetylcholinesterase inhibitor according to the invention is a natural Compound, e.g. Huperzine A, Galantamine, Onchidal, Coumarins, glycoalkaloids, puromycin and cocaine derivatives).

As indicated above, inhibition of AChE may also be effected by lowering the expression of AChE by specifically targeting AChE mRNA molecules. Therefore in other embodiments, the AChE inhibitor according to the invention acts by interacting with the AChE mRNA and may be for example an AChE-specific ribozyme, a double-stranded nucleotide sequence used for RNA interference of the AChE gene, or an antisense oligonucleotide specifically directed against AChE. In more specific embodiments, the AChE inhibitor according to the invention may be an antisense oligonucleotide directed against AChE. The antisense oligonucleotides directed against AChE have been described in the past by the present inventors [WO 03/002739 and U.S. Pat. No. 7,074,915]. An antisense oligonucleotide is an oligonucleotide comprising a sequence which is complementary to the nucleic acid sequence of another (sense) oligonucleotide. For example, an antisense DNA molecule (strand) serves as a template for mRNA synthesis, thus providing an mRNA molecule which is essentially identical to the sense DNA molecule (strand) which encodes a protein.

In yet further embodiments, the AChE inhibitor according to the invention is an antisense oligonucleotide directed against AChE having the nucleic acid sequence of:

### 5′ CTGCCACGTTCTCCTGCACC 3′ (SEQ ID NO. 53); or

5′ CTGCCACGTTCTCCTGCA*C*C*3′ (SEQ ID NO. 54), wherein the three 3′ terminal residues are modified with 2-O-methyl groups (*). These antisense oligonucleotides are also referred to herein as hEN101.

It should be appreciated that all antagonists described herein are applicable in any of the methods and kits of the invention.

According to certain embodiments, the methods of the invention are applicable for AChE-signaling associated disorders that are further characterized with at least one of elevated blood pressure (diastolic and systolic), reduced cortisol levels, elevated anxiety (trait and state), reduced cortisol levels and elevated inflammation.

The phrase “AChE-associated biological pathway”, “AChE-signaling associated disorder” refers to any biological pathway (and the associated disorders) which involves, is regulated by, stimulated by, and/or results from acetylcholinesterase (AChE). Non-limiting examples of such biological pathways include various cholinergic signaling pathways and cross-signaling pathways, specifically, aberrant cholinergic signaling, abnormal hematopoietic proliferation and/or differentiation, cellular stress, exposure to inflammatory response-inducing agents, and/or exposure to organophosphates or to dopaminergic neurotoxin, embryonic development, nervous system development, retina development, neoplasma, neurodegeneration, hematopoiesis, megakaryocyte proliferation and/or differentiation, neuronal cell differentiation, apoptosis, stress reactions and immune reaction.

In more specific embodiments these disorders may be any one of anxiety, hypertension and any immune-related disorders, specifically, inflammatory disorders and proliferative disorders. It should be understood that the methods of the invention further relates to any related conditions of the described pathologies.

As shown by the Examples, patients displaying at least one SNP as described by the invention are also correlated with elevated blood pressure and reduced cortisol levels. These symptom also correlate with anxiety. Therefore, according to some embodiments, the diagnostic, prognostic and therapeutic methods of the invention are applicable for anxiety.

As known in the art, anxiety is generally defined an unpleasant state or condition of inner turmoil, often accompanied by somatic, cognitive, and behavioral elements. Therefore the term “Anxiety disorder” as defined herein is an umbrella term that covers several different forms of a type of common psychiatric disorder characterized by excessive worrying, uneasiness, apprehension and fear concerning future uncertainties either based on real or imagined events, which may affect both physical and psychological health. Anxiety disorders as a collective entity include generalized anxiety disorder (GAD), social phobia (SP), obsessive compulsive disorder (OCD), panic disorder (PD), post-traumatic stress disorder (PTSD) and acute stress disorder. Anxiety disorders present with a marked element of psychological tension and distress and are accompanied by a range of somatic symptoms such as palpitations, shortness of breath, dizziness, hyperthermia, and digestive disturbance. For example, classifying mental disorders may be performed using the Diagnostic and Statistical Manual of Mental Disorders (DSM) published by the American Psychiatric Association, which provides a common language and standard criteria for classifying mental disorders.

The term “generalized anxiety disorder” (GAD) as herein defined refers to an anxiety disorder that is characterized by excessive, uncontrollable and often irrational worry about everyday things that is disproportionate to the actual source of worry. For diagnosis of this disorder, symptoms must last at least 6 months. Individuals suffering from GAD often exhibit a variety of physical symptoms, including fatigue, headaches, nausea, numbness in hands and feet, muscle tension, muscle aches, difficulty in swallowing, bouts of difficulty in breathing, difficulty in concentrating, trembling, twitching, irritability, agitation, seating, restlessness, insomnia (i.e. sleeplessness), hot flashes, and rashes and inability to fully control the anxiety.

The term “social anxiety phobia” (SAP) also known as social phobia (SP) as herein defined refers to an anxiety disorder which is one of the most common psychiatric disorders, with a lifetime prevalence of 12%. It is characterized by intense fear in social situations, causing considerable distress and impaired ability to function in at least some parts of daily life. The diagnosis of social anxiety disorder can be of a specific disorder (when only specific social situations are feared) or a generalized disorder. Generalized social anxiety disorder typically involves a persistent, intense, chronic fear of being judged by others and of being embarrassed or humiliated by one's own actions.

The term “obsessive-compulsive disorder” (OCD) is herein defined is an anxiety disorder characterized by intrusive (involuntary) thoughts that produce uneasiness, apprehension, fear, or worry; by repetitive behaviors aimed at reducing the associated anxiety; or by a combination of such obsessions and compulsions. Symptoms of the disorder include excessive washing or cleaning; repeated checking; preoccupation with sexual, violent or religious thoughts; relationship-related obsessions; aversion to particular numbers; and nervous rituals. The acts of those who have OCD may appear paranoid and potentially psychotic. Obsessive-compulsive disorder affects children and adolescents as well as adults. Roughly one third to one half of adults with OCD report a childhood onset of the disorder, suggesting the continuum of anxiety disorders across the life span.

The term “panic disorder” (PD) as herein defined is an anxiety disorder characterized by sudden attacks of intense fear or anxiety, usually associated with numerous physical symptoms such as heart palpitations, rapid breathing or shortness of breath, blurred vision, dizziness, and racing thoughts. Often these symptoms are thought to be a heart attack by the individual, and many cases are diagnosed in hospital emergency rooms. The symptoms of this disorder come on rapidly and without an identifiable stressor. The individual may have had periods of high anxiety in the past, or may have been involved in a recent stressful situation. The underlying causes, however, are typically subtle.

The term “post-traumatic stress disorder” (PTSD) as herein defined refers to an anxiety disorder that follows a traumatic event which causes intense fear and/or helplessness in an individual. Typically the symptoms develop shortly after the event, but may take years. The duration for symptoms is at least one month for this diagnosis. Symptoms include re-experiencing the trauma through nightmares, obsessive thoughts, and flashbacks (feeling as if you are actually in the traumatic situation again). There is an avoidance component as well, where the individual avoids situations, people, and/or objects which remind him or her about the traumatic event. Finally, there is increased anxiety in general, possibly with a heightened startle response (e.g., very jumpy, startle easy by noises).

The term “acute stress disorder” as herein defined refers to an anxiety disorder which is a result of a traumatic event in which the person experienced or witnessed an event that involved threatened or actual serious injury or death and responded with intense fear and helplessness. Symptoms include dissociative symptoms such as numbing, detachment, a reduction in awareness of the surroundings, de-realization, or depersonalization; re-experiencing of the trauma, avoidance of associated stimuli, and significant anxiety, including irritability, poor concentration, difficulty sleeping, and restlessness. The symptoms must be present for a minimum of two days and a maximum of four weeks and must occur within four weeks of the traumatic event for a diagnosis to be made. The disorder may resolve itself with time or may develop into a more severe disorder such as PTSD.

The psychological phenomenon of anxiety that is experienced by individuals at a certain time (state anxiety) differs from their general susceptibility to anxiety (trait anxiety). Both parameters are commonly measured by the self-reported questionnaires of the state-trait-anxiety inventory (STAI) [8].

In yet another embodiment, the methods of the invention may be applicable for diagnosis, prognosis, assessment and treatment of hypertension. As herein defined the term “hypertension” (HTN) or high blood pressure, sometimes called arterial hypertension, is a chronic medical condition in which the blood pressure in the arteries is elevated. This requires the heart to work harder than normal to circulate blood through the blood vessels. Blood pressure is summarized by two measurements, systolic and diastolic, which depend on whether the heart muscle is contracting (systole) or relaxed between beats (diastole) and equate to a maximum and minimum pressure, respectively. Normal blood pressure at rest is within the range of 100-140 mmHg systolic (top reading) and 60-90 mmHg diastolic (bottom reading). Generally, high blood pressure is said to be present if it is persistently at or above 140/90 mmHg.

Hypertension is classified as either primary (essential) hypertension or secondary hypertension; about 90-95% of cases are categorized as “primary hypertension” which means high blood pressure with no obvious underlying medical cause. Secondary hypertension may be caused by other conditions that affect the kidneys, arteries, heart or endocrine system. Hypertension is a major risk factor for stroke, myocardial infarction (heart attacks), heart failure, aortic aneurysm, peripheral arterial disease and is a cause of chronic kidney disease. Even moderate elevation of arterial blood pressure is associated with a shortened life expectancy.

According to one particular embodiment the present invention further provides method for the diagnosis, prognosis and treatment of an AChE disorder provided that, or with the proviso that said disorder is other than hypertension.

It should be noted that the present invention may be applicable for any cardiovascular disease. As herein defined the term “cardiovascular disease” (also called heart disease) is a class of diseases that involve the heart or blood vessels (arteries, capillaries and veins). Cardiovascular disease refers to any disease that affects the cardiovascular system, principally cardiac disease, vascular diseases of the brain and kidney, and peripheral arterial disease. The causes of cardiovascular disease are diverse but atherosclerosis and/or hypertension are the most common. Cardiovascular disease is the leading cause of deaths worldwide. Although cardiovascular disease usually affects older adults, the antecedents of cardiovascular disease, notably atherosclerosis, begin in early life, rendering primary prevention efforts necessary from childhood.

As shown in Example 6, the SNPs of the invention may predict risk for mal-recovery from AChE associated disorders such as ischemic stroke. Thus, in further embodiments, the methods of the invention may be applicable for diagnosis, prognosis, assessment and treatment of ischemic disorders. The term “ischemia” as herein defined refers to a restriction in blood supply to tissues, causing a shortage of oxygen and glucose needed for cellular metabolism. Ischemia is generally caused by blood vessels problems with resultant damage to or dysfunction of tissue. In some embodiments the ischemic condition is an Ischemic heart disease, e.g. Acute coronary syndrome, Angina pectoris, Angor animi, Coronary artery disease, Coronary ischemia, Hibernating myocardium, Mildronate, Myocardial infarction and Prinzmetal's angina.

In other embodiments the ischemic condition is ischemic stroke. A stroke (also referred to as cerebrovascular accident, CVA), is the rapid loss of brain function due to disturbance in blood supply to the brain. Stroke may be the result of ischemia (lack of blood flow) caused by blockage (which may be the result of thrombosis or arterial embolism). As a consequence, the affected area of the brain cannot function, which might result in an inability to move one or more limbs on one side of the body, among other symptoms. Thus, the term “ischemic stroke” as herein defined refers to an obstruction within a blood vessel supplying blood to the brain. There are various classification systems for acute ischemic stroke, some of them rely primarily on the initial symptoms. Based on the extent of the symptoms, the stroke episode may be classified as total anterior circulation infarct (TACI), partial anterior circulation infarct (PACI), lacunar infarct (LACI) or posterior circulation infarct (POCI). These four entities predict the extent of the stroke, the area of the brain affected, the underlying cause and the prognosis.

As noted above, the invention provides diagnostic and prognostic methods for detecting an AChE-signaling associated disorder, determining genetic predisposition for said disorder, and in some embodiments, further provides methods for the treatment of such disorders. In certain embodiments, the methods of the invention may be applicable for immune-related disorders, specifically, inflammatory disorders. It should be noted that an “Immune-related disorder” is a condition that is associated with the immune system of a subject, either through activation or inhibition of the immune system, or that can be treated, prevented or diagnosed by targeting a certain component of the immune response in a subject, such as the adaptive or innate immune response. In specific embodiments, such disorder may be an inflammatory disease, viral infections, an autoimmune disease, a proliferative disorder or a metabolic disorder.

The terms “inflammatory disease” or “inflammatory-associated condition” refers to any disease or pathologically condition which can benefit from the reduction of at least one inflammatory parameter, for example, induction of an inflammatory cytokine such as IFN-gamma and IL-2 and reduction in IL-6 levels. The condition may be caused (primarily) from inflammation, or inflammation may be one of the manifestations of the diseases caused by another physiological cause.

In yet other specific embodiments the method of the invention may be applicable for a patient suffering from inflammatory bowel diseases (IBD). Inflammatory bowel diseases are common gastrointestinal disorders, that can be perceived as being the result of a dys-balance between the pro- and anti-inflammatory immune responses. IBD is a group of inflammatory conditions of the colon and small intestine. The major types of IBD are Crohn's disease and ulcerative colitis (UC) that share the same symptoms such as diarrhea, vomiting, weight loss, fever and abdominal pain. Other forms of IBD account for far fewer cases. These are Collagenous colitis, Lymphocytic colitis, Ischemic colitis, Diversion colitis, Behçet's syndrome and In-determinate colitis which is an inability to make a definitive diagnosis distinguishing Crohn's disease from Ulcerative colitis.

According to another specific embodiment, the methods of the invention may be applicable for a patient suffering from rheumatoid arthritis. Rheumatoid arthritis (RA) is a chronic, systemic autoimmune disorder that most commonly causes inflammation and tissue damage in joints (arthritis) and tendon sheaths, together with anemia. It can also produce diffuse inflammation in the lungs, pericardium, pleura, and the sclera of the eye, and also nodular lesions, most common in subcutaneous tissue. It can be a disabling and painful condition, which can lead to substantial loss of functioning and mobility.

According to another embodiment, the method of the invention may be used for the diagnosis and the treatment of a patient suffering from a disease defined within the seronegative spondyloarthropathy category, which includes psoriatic arthritis, reactive arthritis, and ankylosing spondylitis, and is characterized by signs of inflammation, multiple joint involvement, and distal involvement in the hands and feet with added features of bone proliferation.

There are many other forms of inflammatory arthritis, including juvenile idiopathic arthritis, gout and pseudo gout, as well as arthritis associated with colitis or psoriasis. It should be therefore understood that the methods of the invention are also applicable for these conditions as well.

The involvement of CDC42 in diabetes type II has been recently reported. Thus, in certain embodiments the method of the invention may be applicable for treating diabetes and any associated conditions. Diabetes mellitus, is a syndrome characterized by disordered metabolism and inappropriately high blood sugar (hyper-glycaemia) resulting from either low levels of the hormone insulin or from abnormal resistance to insulin's effects coupled with inadequate levels of insulin secretion to compensate. The characteristic symptoms are excessive urine production (polyuria), excessive thirst and increased fluid intake (polydipsia), and blurred vision; these symptoms are likely absent if the blood sugar is only mildly elevated. There are three main forms of diabetes: type 1, type 2 and gestational diabetes (occurs during pregnancy).

According to some specific embodiments, the methods of the invention may be applicable for preventing, treating, ameliorating or inhibiting diabetes type II. Diabetes mellitus type 2, or non-insulin-dependent diabetes mellitus (NIDDM) or adult-onset diabetes, is a metabolic disorder that is characterized by high blood glucose in the context of insulin resistance and relative insulin deficiency. Long-term complications from high blood sugar include an increased risk of heart attacks, strokes, amputation, and kidney failure. There are many factors which can potentially give rise to or exacerbate type 2 diabetes. These include obesity, hypertension, elevated cholesterol (combined hyperlipidemia), and with the condition often termed metabolic syndrome (it is also known as Syndrome X, Reavan's syndrome, or CHAOS). Other causes include acromegaly, Cushing's syndrome, thyrotoxicosis, pheochromocytoma, chronic pancreatitis, cancer and drugs. Additional factors found to increase the risk of type 2 diabetes include aging, high-fat diets and a less active lifestyle.

Insulin resistance means that body cells do not respond appropriately when insulin is present. Unlike type 1 diabetes mellitus, insulin resistance is generally “post-receptor”, meaning it is a problem with the cells that respond to insulin rather than a problem with the production of insulin. Severe complications can result from improperly managed type 2 diabetes, including renal failure, erectile dysfunction, blindness, slow healing wounds (including surgical incisions), and arterial disease, including coronary artery disease. The onset of type 2 has been most common in middle age and later life, although it is being more frequently seen in adolescents and young adults due to an increase in child obesity and inactivity. There is growing evidence that there may be a link between inflammation and the pathogenesis of Type 2 diabetes.

In yet other specific embodiments, the methods of the invention may be applicable for preventing, treating, ameliorating or inhibiting diabetes type I. Type 1 diabetes mellitus is characterized by loss of the insulin-producing beta cells of the islets of Langerhans in the pancreas, leading to a deficiency of insulin. The main cause of this beta cell loss is a T-cell mediated autoimmune attack. Type 1 diabetes can affect children or adults and was traditionally termed “juvenile diabetes” as it represents a majority of cases of diabetes affecting children.

In other specific embodiments, the methods of the invention may be applicable for preventing, treating, ameliorating or inhibiting lupus. The term “lupus” as herein defined refers to a chronic inflammatory disease that occurs when the body's immune system attacks self-tissues and organs. Inflammation caused by lupus can affect many different body systems, including the joints, skin, kidneys, blood cells, brain, heart and lungs.

In yet other embodiments, the method of the invention may be also applicable for an infectious disease. More specifically, such infectious disease may be any one of protozoan diseases, viral diseases, bacterial diseases, parasitic diseases, fungal diseases and mycoplasma diseases.

Recent clinical research has indicated the involvement of miR-608 with leukemia. In this connection, it is interesting to note that the inventors have previously showed that certain leukemias associate with excess AChE activity. Thus, the novel identification of specific SNPs that may regulate different targets, including IL-6 and AChE, as disclosed herein, may be specifically applicable for diagnosing and treating hematopoietic proliferative disorders such as leukemia. Accordingly, the methods of the invention may be applicable for diagnosis, prognosis, assessment and treatment of hematopoietic proliferative disorders. The term “hematopoietic cancer” or “hematopoietic proliferative disorder” as herein defined refers to cancer derived from hematopoietic cells, which are immature cells that can develop into all types of blood cells, including white blood cells, red blood cells, and platelets. Hematopoietic stem cells are found in the peripheral blood and the bone marrow. By way of a non-limiting example, an hematopoietic cancer may be Leukemia (e.g. Acute Lymphoblastic Leukemia, Adult; Acute Lymphoblastic Leukemia, Childhood; Acute Myeloid Leukemia, Adult; Acute Myeloid Leukemia, Childhood; Chronic Lymphocytic Leukemia; Chronic Myelogenous Leukemia and Hairy Cell Leukemia), Lymphoma (e.g. AIDS-Related Lymphoma; Cutaneous T-Cell Lymphoma; Hodgkin Lymphoma, Adult; Hodgkin Lymphoma, Childhood; Hodgkin Lymphoma During Pregnancy; Mycosis Fungoides; Non-Hodgkin Lymphoma, Adult; Non-Hodgkin Lymphoma, Childhood; Non-Hodgkin Lymphoma During Pregnancy; Primary Central Nervous System Lymphoma; Sézary Syndrome; T-Cell Lymphoma, Cutaneous; and Waldenström Macroglobulinemia) or the hematopoietic cancer may be Chronic Myeloproliferative Disorders, Langerhans Cell Histiocytosis, Multiple Myeloma/Plasma Cell Neoplasm, Myelodysplastic Syndromes or Myelodysplastic/Myeloproliferative Neoplasms.

As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for patients suffering from any one of non-solid (as described above) as well as solid tumors. It should be further noted that malignancy, as contemplated in the present invention may be any one of carcinomas, melanomas, lymphomas, leukemias, myeloma and sarcomas.

In some other embodiments, the methods of the invention may be applicable for diagnosis, prognosis, assessment and treatment of neurodegenerative disorders. Neurodegenerative diseases are defined as hereditary and sporadic conditions which are characterized by progressive nervous system dysfunction. These disorders are often associated with atrophy of the affected central or peripheral structures of the nervous system. More than 600 disorders afflict the nervous system. Neurodegenerative diseases may be associated with cognition, movement, strength, coordination, or myelin impairment, which are associated with the peripheral nervous system (PNS) or the autonomous nervous system (ANS).

In some embodiments the neurodegenerative disease according to the invention is one of but not limited to Alzheimer's Disease and other dementias, Parkinson's Disease, Brain Cancer, Degenerative Nerve Diseases, Encephalitis, Epilepsy, Genetic Brain Disorders, Head and Brain Malformations, Hydrocephalus, Stroke, Multiple Sclerosis, Amyotrophic Lateral Sclerosis (ALS or Lou Gehrig's Disease), Huntington's Disease, Prion Diseases, Frontotemporal dementia, Dementia with Lewy bodies, Progressive supranuclear palsy, Corticobasal degeneration, Multiple system atrophy, Hereditary spastic paraparesis, Spinocerebellar atrophies, Friedreich's ataxia, Amyloidoses, Metabolic (diabetes) related and Toxin related, Charcot Marie Tooth.

In further specific embodiments, the neurodegenerative disease according to the invention is Alzheimer's disease. The term Alzheimer's disease (also known as Alzheimer disease) as herein defined is the most common form of dementia (a serious loss of global cognitive ability in a previously unimpaired person, beyond what might be expected from normal ageing). Most often, AD is diagnosed in people over 65 years of age. As the disease advances, symptoms include confusion, irritability, aggression, trouble with language, and long-term memory loss.

In yet further embodiments the neurodegenerative disease according to the invention is Parkinson's disease. The term Parkinson's disease as herein defined refers to a progressive disorder of the nervous system that affects movement. It develops gradually, sometimes starting with a barely noticeable tremor in just one hand. But while tremor may be the most well-known sign of Parkinson's disease, the disorder also commonly causes stiffness or slowing of movement.

In further embodiments, the disease according to the invention may be a “familial dysautonomia” (also termed hereditary sensory and autonomic neuropathy, type III). Familial dysautonomia as herein defined is a genetic disorder that affects the development and survival of certain nerve cells. The disorder disturbs cells in the autonomic nervous system, which controls involuntary actions such as digestion, breathing, production of tears, and the regulation of blood pressure and body temperature. It also affects the sensory nervous system, which controls activities related to the senses, such as taste and the perception of pain, heat, and cold.

Problems related to this disorder first appear during infancy. Early signs and symptoms include poor muscle tone (hypotonia), feeding difficulties, poor growth, lack of tears, frequent lung infections, and difficulty maintaining body temperature.

The inventors have previously shown increase in brain AChE in epilepsy. Therefore, in certain embodiments, the method of the invention may be applicable for early diagnosis as well as treatment of epilepsy. As used herein “epilepsy” is a common and diverse set of chronic neurological disorders characterized by seizures. In many cases a cause cannot be identified; however, several of the associated factors include brain trauma, strokes and brain cancer.

In some embodiments, the disease according to the invention may be a muscle disease. The term “muscle disease” (also known as myopathy) is a muscular disease in which the muscle fibers do not function for any one of many reasons, resulting in muscular weakness. This meaning implies that the primary defect is within the muscle, as opposed to the nerves or elsewhere (e.g., the brain etc.). Muscle cramps, stiffness and spasm can also be associated with myopathy. Muscular disease can be classified as neuromuscular or musculoskeletal in nature. Some conditions, such as myositis, can be considered both neuromuscular and musculoskeletal.

In some embodiments, the muscular disease according to the invention may be Acid Maltase Deficiency (AMD), Amyotrophic Lateral Sclerosis (ALS), Andersen-Tawil Syndrome, Becker Muscular Dystrophy (BMD), Becker Myotonia Congenita, Bethlem Myopathy, Bulbospinal Muscular Atrophy (Spinal-Bulbar Muscular Atrophy), Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Muscular Dystrophy (CMD), Congenital Myasthenic Syndromes (CMS), Congenital Myotonic Dystrophy, Cori Disease (Debrancher Enzyme Deficiency), Debrancher Enzyme Deficiency, Dejerine-Sottas Disease (DSD), Dermatomyositis (DM), Distal Muscular Dystrophy (DD), Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Facioscapulohumeral Muscular Dystrophy (FSH or FSHD), Finnish (Tibial) Distal Myopathy, Forbes Disease (Debrancher Enzyme Deficiency), Friedreich's Ataxia (FA), Fukuyama Congenital Muscular Dystrophy, Glycogenosis, Hauptmann-Thanheuser MD (Emery-Dreifuss Muscular Dystrophy), Hereditary Inclusion-Body Myositis, Hyperthyroid Myopathy, Hypothyroid Myopathy, Inclusion-Body Myositis (IBM), Inherited Myopathies, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Limb-Girdle Muscular Dystrophy (LGMD), Lou Gehrig's Disease (Amyotrophic Lateral Sclerosis), McArdle Disease (Phosphorylase Deficiency), Merosin-Deficient Congenital Muscular Dystrophy, Metabolic Diseases of Muscle, Mitochondrial Myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphoglycerate Kinase Deficiency, Phosphoglycerate Mutase Deficiency, Phosphorylase Deficiency, Polymyositis (PM), Pompe Disease (Acid Maltase Deficiency), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia Congenita), Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), and ZASP-Related Myopathy.

In some embodiments, the disease according to the invention is Chronic fatigue syndrome (CFS). CFS is the common name for a group of significantly debilitating medical conditions characterized by persistent fatigue and other specific symptoms that lasts for a minimum of six months in adults (and 3 months in children or adolescents). The fatigue is not due to exertion, not significantly relieved by rest, and is not caused by other medical conditions. CFS may also be referred to as myalgic encephalomyelitis (ME), post-viral fatigue syndrome (PVFS), chronic fatigue immune dysfunction syndrome (CFIDS), or by several other terms. Symptoms of CFS include malaise after exertion; un-refreshing sleep, widespread muscle and joint pain, sore throat, headaches of a type not previously experienced, cognitive difficulties, chronic and severe mental and physical exhaustion, and other characteristic symptoms in a previously healthy and active person. Additional symptoms may be reported, including muscle weakness, depression, cardiac and respiratory problems to name but few.

In some embodiments the disease according to the invention is Myasthenia gravis. The term “Myasthenia gravis” as herein defined is an autoimmune neuromuscular disease leading to fluctuating muscle weakness and fatigability. It is an autoimmune disorder, in which weakness is caused by circulating antibodies that block acetylcholine receptors at the postsynaptic neuromuscular junction, inhibiting the excitatory effects of the neurotransmitter acetylcholine on nicotinic receptors throughout neuromuscular junctions. Myasthenia is treated medically with acetylcholinesterase inhibitors or immunosuppressants, and, in selected cases, thymectomy. The disease incidence is 3-30 cases per million per year and rising as a result of increased awareness. MG must be distinguished from congenital myasthenic syndromes that can present similar symptoms but offer no response to immunosuppressive treatments.

In other embodiments the disease according to the invention is Duchenne's muscular dystrophy (DMD). Duchenne's muscular dystrophy, as herein defined is a recessive X-linked form of muscular dystrophy, affecting around 1 in 3,600 boys, which results in muscle degeneration and eventual death. The disorder is caused by a mutation in the dystrophin gene, located on the human X chromosome, which codes for the protein dystrophin, an important structural component within muscle tissue that provides structural stability to cell membrane. While both sexes can carry the mutation, females rarely exhibit signs of the disease. Symptoms usually appear in male children before age 6 and may be visible in early infancy. Progressive proximal muscle weakness of the legs and pelvis associated with a loss of muscle mass is observed first. Eventually this weakness spreads to the arms, neck, and other areas.

In further embodiments the disease according to the invention may be Glaucoma. The term “Glaucoma” as herein defined refers to an eye disease in which the optic nerve is damaged in a characteristic pattern. This can permanently damage vision in the affected eye(s) and lead to blindness if left untreated. It is normally associated with increased fluid pressure in the eye (aqueous humour). The many different subtypes of glaucoma can all be considered to be a type of optic neuropathy.

In further embodiments the disease according to the invention is Nephritic syndrome. The term “Nephritic syndrome” as herein defined relates to a collection of signs associated with disorders affecting the kidneys (e.g. glomerular disorders). It is characterized by having small pores in the podocytes of the glomerulus, large enough to permit proteins (proteinuria) and red blood cells (hematuria) to pass into the urine. By contrast, nephrotic syndrome is characterized by only proteins (proteinuria) moving into the urine. Both nephritic syndrome and nephrotic syndrome result in hypoalbuminemia due to protein albumin moving from the blood to the urine.

It should be appreciated that all the disorders disclosed herein or any associated condition or pathology are encompassed by the present invention and therefore may be applicable for practicing any of the methods and kits of the invention. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

A further aspect of the invention relates to a method for the diagnosis and prognosis of an AChE-signaling associated disorder in a human subject. Such method comprises the step of: In the first step (a) genotyping in a biological sample of the tested subject at least one SNP in the 3′UTR of the AChE gene, wherein said SNP is at least one of rs17228616, rs17228602, rs17883268, rs17228609, rs17235010, as well as any of the SNPs disclosed in Tables 1 and 2, herein after. The second step (b) involves identifying and/or detecting in the genotyped sample obtained in step (a) at least one SNP that modulates the binding affinity of at least one of miR-608, miR-125b-3p, miR-4319, miR-125a-5p, miR-761, miR-214, miR-298 and miR-4283 to the 3′UTR of the AChE gene.

It should be noted that in certain embodiments, detecting the presence of at least one SNP that modulates the binding affinity of at least one of said miR-608, miR-125b-3p, miR-4319, miR-125a-5p, miR-761, miR-214, miR-298 and miR-4283 to the 3′UTR of the AChE gene indicates that the examined subject belongs to a predetermined population associated with at least one AChE-signaling associated disorder, thereby diagnosing such disorder in the tested subject.

Some embodiments of the invention relate to a method comprising the steps of: In a first step (a) genotyping in a biological sample of the examined subject at least one SNP in the 3′UTR of the AChE gene, said SNP is at least one of rs17228616, rs17228602, rs17883268 and rs17235010. In a second step (b) determining and/or identifying in the genotyped sample obtained in step (a), the presence or the absence of at least one of C2098A of SNP rs17228616, G2165A of SNP rs17228602, T2402C of SNP rs17883268 and G2071A of SNP rs17235010 in at least one allele of the 3′UTR of the AChE gene in the examined sample. In some embodiments, the presence of at least one of A at rs17228616, A at rs17228602, C at rs17883268, A at rs17228609 and A at rs17235010 in at least one allele of the AChE gene indicates that the examined subject belongs to a predetermined population associated with said at least one AChE-signaling associated disorder. Moreover, such method may also provide predicting the risk of a delayed disorder.

As noted before, the specific SNP may modulate, specifically, either decrease or enhance binding of the specific miRNAs indicated by the invention, to the AChE 3′UTR. As a result, the regulation of different target genes by said miRNAs may be disturbed or enhanced thereby leading to aberrant expression of such target genes. Thus, some embodiments of the invention relay on further biological markers that are a result of the specific SNP. These methods therefore further comprise the steps of measuring the level of these markers. Thus, according to certain embodiments the methods of the invention may further comprise the following steps:

A third step (c) involves determining in at least one biological sample of a subject displaying the presence of at least one of A at rs17228616, A at rs17228602, C at rs17883268 and A at rs17235010 in at least one allele of the AChE gene, as identified in step (b), at least one of:

(i) at least one of the level of expression and the catalytic activity of AChE in at least sample, to obtain at least one of an expression value or an activity value of AChE. According to certain embodiments, the level of expression and the expression and catalytic activity of BChE is also identified and compared to the activity of AChE. According to some embodiments BChE may serve as a control.
(ii) the expression level of at least one gene target of at least one of miR-608, miR-125b-3p, miR-4319, miR-125a-5p, miR-761, miR-214, miR-298 and miR-4283 in at least one sample to obtain an expression value of such at least one gene target.

The next step (d) involves determining at least one of:

(i) if the expression or the activity value of AChE obtained in step (ci) is any one of, positive or negative with respect to a predetermined standard expression value or activity value of the AChE or to an expression value or activity value of AChE in at least one control sample. In certain embodiments where the expression and catalytic activity of BChE is also identified as a control, this step further involves determining if the expression and activity values of BChE obtained in step (ci) is any one of, positive or negative with respect to a predetermined standard expression value or activity value of said BChE or to an expression value or activity value of AChE in at least one control sample.
(ii) determining if the expression value of at least one gene target of at least one of miR-608, miR-125b-3p, miR-4319, miR-125a-5p, miR-761, miR-214, miR-298 and miR-4283 obtained in step (cii) is any one of, positive or negative with respect to a predetermined standard expression value of said at least one gene target of at least one of miR-608, miR-125b-3p, miR-4319, miR-125a-5p, miR-761, miR-214, miR-298 and miR-4283, or to an expression value of said target genes in at least one control sample.

In certain embodiments, when the specific SNP leads to reduction of the binding affinity of a specific relevant miRNA to the AChE 3′UTR, an elevated expression of AChE followed by reduced expression of other gene targets of the same specific miRNA, may be expected. In such embodiments, at least one of, a positive expression or activity value of said AChE and a negative expression value of at least one gene target of at least one of miR-608, miR-125b-3p, miR-4319, miR-125a-5p, miR-761, miR-214, miR-298 and miR-4283, indicates that said subject belongs to a predetermined population associated with said at least one AChE-signaling associated disorder. In yet other alternative embodiments, where the specific SNP leads to enhancement in the binding affinity of a specific relevant miRNA to the AChE 3′UTR, one can expect an elevated suppression of AChE followed by reduced expression thereof, and enhanced expression of other gene targets of the same specific miRNA. In such embodiments, at least one of, a negative expression or activity value of said AChE and a positive expression value of at least one gene target of at least one of miR-608, miR-125b-3p, miR-4319, miR-125a-5p, miR-761, miR-214, miR-298 and miR-4283, indicates that said subject belongs to a predetermined population associated with said at least one AChE-signaling associated disorder. In cases the expression and activity values of BChE are determined, the values of said marker are within the same range in population of patients and in healthy population, and therefore, BChE may serve as a control.

It should be noted that the invention encompasses the use of any of the gene targets of any one of miR-608, miR-125b-3p, miR-4319, miR-125a-5p, miR-761, miR-214, miR-298 and miR-4283, as biomarkers for monitoring aberrant regulation of the miRNA targets caused by different SNPs. Non-limiting examples for such target genes include interleukin-6 (IL-6), CDC42, CDC44, interleukin-1 alpha (IL1A), growth hormone receptor (GHR), and TP53, that are targets of miR-608; targets of miR-125b-3p that may be p53, MUC1 and TNF-α; LIN28A, NTRK3 and CD34 that are targets of miR-125a-5p; APOBEC2, and CCDC21 that are targets of miR-761; the miR-298 target BACE1 and the targets of miR-4283 that include SIGLEC8, GNG13, and SENP1.

It should be noted that the diagnostic and prognostic method of the invention may further combine examination of standard parameters that are currently used for evaluating and diagnosing different AChE-associated disorders. Such combination may increase sensitivity and may provide an early diagnosis, even before the appearance of the clinical symptoms. Thus, according to certain embodiments the methods of the invention may further comprise the steps of: determining in at least one biological sample of a subject displaying the presence of at least one of A at rs17228616, A at rs17228602, C at rs17883268 and A at rs17235010 in at least one allele of the AChE gene, as identified in step (b), at least one of blood pressure (diastolic and systolic), anxiety (state and trait), serum levels of TNF-α, serum levels of C reactive protein (CRP) and serum cortisol levels.

It should be noted that cortisol as the primary glucocorticoid hormone of the human body exerts multiple regulatory functions in almost every single tissue. Changes in the secretory activity of the adrenal cortex (either hypo- or hyper activity) are known to be associated with adverse health outcomes. It is thus mandatory to monitor changes of HPA functioning in individuals exposed to chronic or traumatic stress. While corticotropin-releasing hormone (CRH) and adrenocorticotropin hormone (ACTH) can have multiple effects in the brain, cortisol appears to be the best candidate to date for the assessment of long-term stress effects on the brain and body.

According to some embodiments, the method of the invention may further comprise the step of determining the ethnic group of said subject, wherein said ethnic group may be any one of African, Caucasian, Ashkenazi Jews, Sepharadic Jews and Israeli Arabs.

According to certain embodiments, the diagnostic methods described by this aspect of the invention may further provide information regarding the selection of the appropriate population of patients that may be treated using a specific therapeutic regimen. Such method may be therefore further combined with a therapeutic step. According to these embodiments, the methods of the invention may further comprise the step of administering to a subject diagnosed with said at least one AChE-signaling associated disorder (according to the diagnostic method of the invention), a therapeutic effective amount of at least one of: (i) at least one antagonist of at least one of miR-608, miR-125b-3p, miR-4319, miR-125a-5p, miR-761, miR-214, miR-298 and miR-4283; and (ii) at least one AChE antagonist.

It should be further understood that since the specific SNPs modulate the regulation of different miRNA gene targets, the therapeutic regimens disclosed by the invention may further encompass the use of at least one agent that regulates (either potentiates or eliminates) the expression of at least one other target of said miR whose binding is modified by the AChE SNP.

According to certain specific embodiments that concern SNPs that disturb the regulation of miR-608, the method of the invention comprises the steps of: First step (a), involves genotyping in a biological sample of the examined subject SNP rs17228616. The second step (b) concerns determining or identifying the presence or the absence of C2098A of SNP rs17228616 in at least one allele of the AChE gene in the genotyped sample obtained in step (a).

The presence of A allele at rs17228616, or more specifically, the presence of adenine in position 2098 of rs17228616 in at least one allele of the AChE gene, indicates that the tested subject belongs to a predetermined population associated with at least one AChE-signaling associated disorder.

Still further, in some embodiments, the method further comprises:

A third step (c) that involves determining in at least one biological sample of a subject displaying the presence of A at rs17228616 in at least one allele of the AChE gene, as identified in step (b), at least one of:

(i) at least one of the level of expression and the catalytic activity of AChE in said sample, to obtain at least one of an expression value or an activity value of said AChE.
(ii) the expression level of at least one gene target of miR-608 in said sample to obtain an expression value of said at least one gene target. Specific examples of such targets include but are not limited to IL-6, CDC42, CDC44, interleukin-1 alpha (IL1A), growth hormone receptor (GHR), and TP53.

According to certain embodiments, the level of expression and the expression and catalytic activity of BChE is also identified and compared to the activity of AChE.

The next step (d) involves determining at least one of:

(i) if the expression or the activity value of AChE obtained in step (ci) is any one of, positive or negative with respect to a predetermined standard expression value or activity value, or to an expression value or activity value of AChE in at least one control sample; and
(ii) determining if the expression value of at least one gene target of miR-608 obtained in step (cii) is any one of, positive or negative with respect to a predetermined standard expression value of said at least one gene target of miR-608, or to an expression value of such target genes in at least one control sample. In certain embodiments where the expression and catalytic activity of BChE is also identified, this step further involves determining if the expression and activity values of BChE obtained in step (ci) is any one of, positive or negative with respect to a predetermined standard expression value or activity value of said BChE or to an expression value or activity value of AChE in at least one control sample.

It should be noted that wherein at least one of, a positive expression or activity value of AChE and a negative expression value of at least one gene target of miR-608, indicates that the subject belongs to a predetermined population associated with said at least one AChE-signaling associated disorder. In cases the expression and activity values of BChE are determined, the values of this marker are within the same range in population of patients and in healthy population, and therefore, BChE may serve as a control.

According to some specific embodiments, the target of miR-608 may be at least one of CDC42 and IL-6. According to these specific embodiments the following steps are involved: First step (a) involves genotyping in a biological sample of said subject SNP rs17228616. In the next step (b) determining and/or identifying the presence or the absence of C2098A of SNP rs17228616 in at least one allele of the AChE gene in the genotyped sample obtained in step (a). The third step (c) concerns determining in at least one biological sample of a subject displaying the presence of A at rs17228616 in at least one allele of the AChE gene, as identified and/or determined in step (b), at least one of:

(i) at least one of the level of expression and the catalytic activity of AChE in said sample, to obtain at least one of an expression value or an activity value of said AChE;
(ii) determining the expression level of at least one of CDC42 and IL-6 in said sample to obtain an expression value of said at least one of CDC42 and IL-6 in said sample;

According to certain embodiments, the level of expression and the expression and catalytic activity of BChE is also identified and compared to the activity of AChE.

The next step (d) involves determining at least one of:

(i) if the expression or the activity value of AChE obtained in step (ci) is any one of, positive or negative with respect to a predetermined standard expression value or activity value of AChE, or to an expression value or activity value of AChE in at least one control sample; and
(ii) determining if the expression value of at least one of CDC42 and IL-6 obtained in step (cii) is any one of, positive or negative with respect to a predetermined standard expression value of said at least one of CDC42 and IL-6 or to the expression value of said at least one of CDC42 and IL-6 in at least one control sample;

In certain embodiments where the expression and catalytic activity of BChE is also identified, this step further involves determining if the expression and activity values of BChEA obtained in step (ci) is any one of, positive or negative with respect to a predetermined standard expression value or activity value of said BChE or to an expression value or activity value of AChE in at least one control sample. It should be noted that wherein at least one of a positive expression or activity value of said AChE (but not BChE) and a negative expression value of at least one of CDC42 and IL-6 indicates that said subject belongs to a predetermined population associated with said AChE-signaling associated disorders. In cases the expression and activity values of BChE are determined, the values of said marker are within the same range in population of patients and in healthy population, and therefore, BChE or the ratio between AChE and BChE may serve as a control.

In certain embodiments, the methods of the invention further comprise the steps of: determining in at least one biological sample of a subject displaying the presence of at least one of A at rs17228616, A at rs17228602, C at rs17883268, A at rs17228609 and A at rs17235010 in at least one allele of the AChE gene, as identified in step (b), at least one of blood pressure (diastolic and systolic), anxiety (state and trait), serum levels of TNF-α, serum levels of C reactive protein (CRP) and serum cortisol levels.

For diagnosing patients suffering from an AChE-associated disorder, the method of the invention involves in step (c) thereof, determining the expression level of a specific biomarker, that may be at least one of AChE and of at least one miR target gene (specifically, any one of the target genes disclosed by the invention herein before), in a sample. Moreover, where other parameters are also examined as discussed above, the levels of for example, CRP, TNF-α and cortisol, may also be determined as described herein. These molecules may also be referred to by the invention as suitable additional biomarkers that may be combined with the biomarkers of the invention.

The terms “level of expression” or “expression level” are used interchangeably and generally refer to the amount of a polynucleotide or a protein in a biological sample. “Expression” generally refers to the process by which gene-encoded information is converted into the structures present and operating in the cell. Therefore, according to the invention “expression” of a gene, specifically, a gene encoding at least one of AChE and of at least one miR target may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis.

It should be noted that the expression level is reflected by measurement and determination of an expression value. As used herein, the term “expression value”, “level of expression” or “expression level” refers to numerical representation of a quantity of a gene product, which herein is a protein, but may also be an mRNA.

In order to perform the steps of determining the expression level of at least one of AChE and of at least one miR target gene, the method of the invention further involves contacting the biological sample or any protein or nucleic acid product obtained therefrom with detecting molecules specific for said at least one of AChE and of at least one miR target gene and optionally for at least one reference control, e.g., BChE. More specifically, the method of invention relies on the detection of at least one of AChE and of at least one miR target gene by contacting detecting molecules specific for at least one of AChE and of at least one miR target gene) with a biological sample. The term “contacting” means to bring, put, incubates or mixes together. As such, a first item is contacted with a second item when the two items are brought or put together, e.g., by touching them to each other or combining them. In the context of the present invention, the term “contacting” includes all measures or steps, which allow interaction between at least one detecting molecule specific for at least one of AChE and of at least one miR target gene (for example, CDC42 and IL-6) and the tested sample, or any component derived therefrom. In certain embodiments where reference controls are also used, for example, BChE, the method of the invention further involves contacting steps, which allow interaction between at least one detecting molecule specific for a suitable reference control and the tested sample or any component derived therefrom. The contacting is performed in a manner by which the at least one of detecting molecule of at least one of AChE and of at least one miR target gene and at least one suitable reference control (BChE) can interact with or bind to the peptide molecules or nucleic acid molecule in the tested sample. The binding will preferably be non-covalent, reversible binding, e.g., binding via salt bridges, hydrogen bonds, hydrophobic interactions or a combination thereof.

It should be appreciated that determination of the level of at least one of AChE and of at least one miR target gene (CDC42 and IL-6) expression in the biological sample can be effected at the transcriptional level (i.e., mRNA) using detecting molecules that are based on nucleic acids (an oligonucleotide probe or primer), or alternatively, at the translational level (i.e. protein level or activity, such as enzymatic activity) using amino acid or substrate-hydrolysis based detecting molecules, as also demonstrated by the present invention. Thus, according to one specific embodiment, the detecting molecules used by the methods of the invention may be isolated detecting amino acid molecules or isolated detecting nucleic acid molecules, or any combinations thereof.

According to one specific embodiment, the detection of at least one of AChE and of at least one miR target gene expression can be affected at the protein level. Therefore, the detecting molecules used by the method of the invention may be amino acid molecules, specifically, an isolated antibodies that specifically recognize and binds said markers (at least one of AChE and of at least one miR target gene).

As indicated above, the detecting molecules of the invention may be amino acid based molecules that may be referred to as protein/s or polypeptide/s. As used herein, the terms “protein” and “polypeptide” are used interchangeably to refer to a chain of amino acids linked together by peptide bonds. It should be noted that peptide bond as described herein is a covalent amid bond formed between two amino acid residues.

The invention further contemplates the use of amino acid based molecules such as proteins or polypeptides as detecting molecules disclosed herein and would be known by a person skilled in the art to measure the protein marker of the invention, specifically, at least one of AChE and of at least one miR target gene. Techniques known to persons skilled in the art (for example, techniques such as Western Blotting, Immunoprecipitation, ELISAs, protein microarray analysis, Flow cytometry and the like) can then be used to measure the level of protein products corresponding to the biomarker of the invention. As would be understood to a person skilled in the art, the measure of the level of expression of the protein products of the biomarkers of the invention requires a protein, which specifically and/or selectively binds to the biomarker of the invention.

In specific embodiments, the detecting amino acid molecules may be isolated antibodies, with specific binding selectively to at least one of AChE and of at least one miR target gene. Using these antibodies, the level of expression of said at least one marker gene may be determined using an immunoassay which may be any one of FACS, a Western blot, an ELISA, a RIA, a slot blot, a dot blot, immunohistochemical assay, immunofluorescent assay and a radio-imaging assay.

The term “antibody” as used in this invention includes whole antibody molecules as well as functional fragments thereof, such as Fab, F(ab′)2, and Fv that are capable of binding with antigenic portions of the target polypeptide, i.e. at least one of AChE and of at least one miR target gene (. The antibody is preferably mono-specific, e.g., a monoclonal antibody, or antigen-binding fragment thereof. The term “mono-specific antibody” refers to an antibody that displays a single binding specificity and affinity for a particular target, e.g., epitope. This term includes a “monoclonal antibody” or “monoclonal antibody composition”, which are used herein refer to a preparation of antibodies or fragments thereof of single molecular composition.

It should be recognized that the antibody can be a human antibody, a chimeric antibody, a recombinant antibody, a humanized antibody, a monoclonal antibody, or a polyclonal antibody. The antibody can be an intact immunoglobulin, e.g., an IgA, IgG, IgE, IgD, 1 gM or subtypes thereof. The antibody can be conjugated to a functional moiety (e.g., a compound which has a biological or chemical function. The antibody used by the invention interacts with a polypeptide that is at least one of AChE and of at least one miR target gene, with high affinity and specificity. As noted above, the term “antibody” also encompasses antigen-binding fragments of an antibody. The term “antigen-binding fragment” of an antibody (or simply “antibody portion,” or “fragment”), as used herein, may be defined as follows:

(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, defined as a genetically engineered fragment containing the variable region of the light chain and the variable region of the heavy chain expressed as two chains; and
(5) Single chain antibody (“SCA”, or ScFv), a genetically engineered molecule containing the variable region of the light chain and the variable region of the heavy chain, linked by a suitable polypeptide linker as a genetically fused single chain molecule.

Methods of generating such antibody fragments are well known in the art (See for example, Harlow and Lane, Antibodies: A Laboratory Manual, Cold Spring Harbor Laboratory, New York, 1988, incorporated herein by reference).

Still further, for determination and monitoring uses described herein after, the antibodies specific for at least one of AChE and of at least one miR target gene and optionally for the reference controls (BChE, GAPDP) used by the present invention may optionally be covalently or non-covalently linked to a detectable label. The term “labeled” can refer to direct labeling of the antibody via, e.g., coupling (i.e., physically linking) a detectable substance to the antibody, and can also refer to indirect labeling of the antibody by reactivity with another reagent that is directly labeled.

Means of detecting such labels are well known to those of skill in the art. Thus, for example, radiolabels may be detected using photographic film or scintillation counters, fluorescent markers may be detected using a photo-detector to detect emitted illumination. Enzymatic labels are typically detected by providing the enzyme with a substrate and detecting the reaction product produced by the action of the enzyme on the substrate, and colorimetric labels are detected by simply visualizing the colored label.

The antibody used as a detecting molecule according to the invention, specifically recognizes and binds said marker (specifically, at least one of AChE and of at least one miR target gene). It should be therefore noted that the term “binding specificity”, “specifically binds to an antigen”, “specifically immuno-reactive with”, “specifically directed against” or “specifically recognizes”, when referring to an epitope, specifically, a recognized epitope within the marker molecule (specifically, at least one of AChE and of at least one miR target gene), refers to a binding reaction which is determinative of the presence of the epitope in a heterogeneous population of proteins and other biologics. More particularly, “selectively bind” in the context of proteins encompassed by the invention refers to the specific interaction of any two of a peptide, a protein, a polypeptide an antibody, wherein the interaction preferentially occurs as between any two of a peptide, protein, polypeptide and antibody preferentially as compared with any other peptide, protein, polypeptide and antibody. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

According to one embodiment, where amino acid-based detection molecules are used, the expression level of the marker protein, in the tested sample can be determined using different methods known in the art, specifically method disclosed herein below as non-limiting examples.

### Enzyme-Linked Immunosorbent Assay (ELISA)

This method involves fixation of a sample containing a protein substrate (e.g., fixed cells or a proteinaceous solution) to a surface such as a well of a microtiter plate. A substrate-specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

### Western Blot

This method involves separation of a substrate from other protein by means of an acrylamide gel followed by transfer of the substrate to a membrane (e.g., nitrocellulose, nylon, or PVDF). Presence of the substrate is then detected by antibodies specific to the substrate, which are in turn detected by antibody-binding reagents. Antibody-binding reagents may be, for example, protein A or secondary antibodies. Antibody-binding reagents may be radiolabeled or enzyme-linked, as described hereinafter. Detection may be by autoradiography, colorimetric reaction, or chemiluminescence. This method allows both quantitation of an amount of substrate and determination of its identity by a relative position on the membrane indicative of the protein's migration distance in the acrylamide gel during electrophoresis, resulting from the size and other characteristics of the protein.

### Radioimmunoassay (RIA)

In one version, this method involves precipitation of the desired protein (i.e., the substrate) with a specific antibody and radiolabeled antibody-binding protein (e.g., protein A labeled with I¹²⁵) immobilized on a precipitable carrier such as agarose beads. The radio-signal detected in the precipitated pellet is proportional to the amount of substrate bound.

In an alternate version of RIA, a labeled substrate and an un-labeled antibody-binding protein are employed. A sample containing an unknown amount of substrate is added in varying amounts. The number of radio counts from the labeled substrate-bound precipitated pellet is proportional to the amount of substrate in the added sample.

### Fluorescence-Activated Cell Sorting (FACS)

This method involves detection of a substrate in situ in cells bound by substrate-specific, fluorescently labeled antibodies. The substrate-specific antibodies are linked to fluorophores. Detection is by means of a flow cytometry machine, which reads the wavelength of light emitted from each cell as it passes through a light beam. This method may employ two or more antibodies simultaneously, and is a reliable and reproducible procedure used by the present invention.

### Immunohistochemical Analysis

This method involves detection of a substrate in situ in fixed cells by substrate-specific antibodies. The substrate specific antibodies may be enzyme-linked or linked to fluorophores. Detection is by microscopy, and is either subjective or by automatic evaluation.

According to one specific embodiment, the detection of at least one of AChE and of at least one miR target gene expression can be affected at the nucleic acid level. Thus, the detecting molecules for assessing the expression of at least one of the biomarkers of the invention (AChE and of at least one miR target gene), may be isolated detecting nucleic acid molecules. According to some embodiments, such detecting nucleic acid molecules may be isolated oligonucleotides, each oligonucleotide specifically hybridizes to a nucleic acid sequence of the RNA products of said at least one of AChE and of at least one miR target gene. More specifically, the oligonucleotide used as a detecting molecule may be any one of a pair of primers or nucleotide probe. In such case, the level of expression of said markers may be determined using a nucleic acid amplification assay selected from the group consisting of: a Real-Time PCR, micro arrays, PCR, in situ Hybridization and Comparative Genomic Hybridization. It should be noted that in particular embodiments, the invention further encompasses the use of aptamers as a nucleic acid based detection molecules that specifically recognize and bind the marker protein.

As used herein, “nucleic acids” is interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. It should be noted that the nucleic acid sequences herein recited are depicted in the 5′ to 3′ direction (from the 5′ terminus to the 3′ terminus), unless otherwise stated.

As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from about 8 to about 1,000 nucleotides long. More specifically, the detecting oligonucleotides molecule used by the composition of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 bases in length.

Different methods of determining levels of the expression of the biomarkers of the invention (specifically, at least one of AChE and of at least one miR target gene) (i.e., RNA or protein) in biological samples using nucleic acid base detection methods may be applicable for performing the method of the invention. Such procedures include, but are not limited to, Northern Blot Analysis, Polymerase chain reaction (PCR)-based methods (e.g., RT-PCR, using oligonucleotide primers or probes as the detection molecules), RNA In Situ Hybridization Stain, In Situ RT-PCR Stain, and Oligonucleotide Microarray procedures (for example, the Affymetrix® GeneChip® Microarray).

More specifically, these methods use nucleic acid based detecting molecules, for example, oligonucleotides that specifically hybridize to nucleic acid sequences of these marker genes. As used herein, the term “hybridize” refers to a process where two complementary nucleic acid strands anneal to each other under appropriately stringent conditions.

As used herein “selective or specific hybridization” in the context of this invention refers to a hybridization which occurs between a polynucleotide encompassed by the invention as detecting molecules, and any one of the marker genes any control reference gene, wherein such hybridization is such that the polynucleotide binds to at least one of the marker genes of the invention, or to any control reference gene preferentially to any other RNA in the tested sample. A polynucleotide which “selectively hybridizes” is one which hybridizes with a selectivity of greater than 60%, and even more, specifically, 100%. The terms, “specifically hybridizes”, “specific hybridization” refers to hybridization which occurs when two nucleic acid sequences are substantially complementary (at least about 60% complementary over a stretch of at least 5 to 25 nucleotides, preferably at least about 70%, 75%, 80% or 85% complementary, more preferably at least about 90% complementary, and most preferably, about 95% complementary).

The measuring of the expression of any one of the markers of the invention and any control reference gene and combination thereof can be done by using those polynucleotides as detecting molecules, which are specific and/or selective for said at least one marker gene or any control reference gene to quantitate expression thereof or of any control reference gene. In a specific embodiment of the invention, such polynucleotides may be probes or primers. It should be further appreciated that the methods, as well as the compositions and kits of the invention may comprise, as an oligonucleotide-based detection molecule, both primers and probes.

The term, “primer”, as used herein refers to an oligonucleotide, whether occurring naturally as in a purified restriction digest, or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 10-30 or more nucleotides, although it may contain fewer nucleotides. More specifically, the primer used by the methods, as well as the kits of the invention may comprise 10, 11, 12, 13, 14, 15, 16, 17, 18, 19, 20, 21, 22, 23, 24, 25, 26, 27, 28, 29 or 30 nucleotides or more.

As used herein, the term “probe” means oligonucleotides and analogs thereof and refers to a range of chemical species that recognize polynucleotide target sequences through hydrogen bonding interactions with the nucleotide bases of the target sequences. The probe or the target sequences may be single- or double-stranded RNA or single- or double-stranded DNA or a combination of DNA and RNA bases. A probe may be 5, 6, 7, 8, 9, 10, 11, 12, 13, 14, 15, 16, 17, 18, 19, 20, 21, 22, 23, 24, 25, 26, 27, 28, 29 and up to 30 nucleotides in length as long as it is less than the full length of the target marker or any gene encoding the same. Probes can include oligonucleotides modified so as to have a tag which is detectable by fluorescence, chemiluminescence and the like. The probe can also be modified so as to have both a detectable tag and a quencher molecule, for example TaqMan® and Molecular Beacon® probes, that will be described in detail below.

Thus, according to one embodiment, the level of expression of at least one of the markers of the invention, is determined using a nucleic acid amplification assay selected from the group consisting of: a Real-Time PCR, micro array, PCR, in situ hybridization and comparative genomic hybridization.

The term “amplify”, with respect to nucleic acid sequences, refers to methods that increase the representation of a population of nucleic acid sequences in a sample. Nucleic acid amplification methods, such as PCR, isothermal methods, rolling circle methods, etc., are well known to the skilled artisan. More specifically, as used herein, the term “amplified”, when applied to a nucleic acid sequence, refers to a process whereby one or more copies of a particular nucleic acid sequence is generated from a template nucleic acid, preferably by the method of polymerase chain reaction. “Polymerase chain reaction” or “PCR” refers to an in vitro method for amplifying a specific nucleic acid template sequence. The PCR reaction involves a repetitive series of temperature cycles and is typically performed in a volume of 50-100 μL. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers”, “a set of PCR primers” or “pair of primers” can comprise two, three, four or more primers.

Real time nucleic acid amplification and detection methods are efficient for sequence identification and quantification of a target since no pre-hybridization amplification is required. Amplification and hybridization are combined in a single step and can be performed in a fully automated, large-scale, closed-tube format.

Methods that use hybridization-triggered fluorescent probes for real time PCR are based either on a quench-release fluorescence of a probe digested by DNA Polymerase (e.g., methods using TaqMan®, MGB-TaqMan®), or on a hybridization-triggered fluorescence of intact probes (e.g., molecular beacons, and linear probes). In general, the probes are designed to hybridize to an internal region of a PCR product during annealing stage (also referred to as amplicon). For those methods utilizing TaqMan® and MGB-TaqMan® the 5′-exonuclease activity of the approaching DNA Polymerase cleaves a probe between a fluorophore and a quencher, releasing fluorescence.

Thus, a “real time PCR” assay provides dynamic fluorescence detection of amplified markers or any control reference gene in a PCR amplification reaction. During PCR, the amplified products created using suitable primers hybridize to probe nucleic acids (TaqMan® probe, for example), which may be labeled according to some embodiments with both a reporter dye and a quencher dye. When these two dyes are in close proximity, i.e. both are present in an intact probe oligonucleotide, the fluorescence of the reporter dye is suppressed. However, a polymerase, such as AmpliTaq Gold™, having 5′-3′ nuclease activity can be provided in the PCR reaction. This enzyme cleaves the fluorogenic probe if it is bound specifically to the target nucleic acid sequences between the priming sites. The reporter dye and quencher dye are separated upon cleavage, permitting fluorescent detection of the reporter dye. Upon excitation by a laser provided, e.g., by a sequencing apparatus, the fluorescent signal produced by the reporter dye is detected and/or quantified. The increase in fluorescence is a direct consequence of amplification of target nucleic acids during PCR. The method and hybridization assays using self-quenching fluorescence probes with and/or without internal controls for detection of nucleic acid application products are known in the art, for example, U.S. Pat. Nos. 6,258,569; 6,030,787; 5,952,202; 5,876,930; 5,866,336; 5,736,333; 5,723,591; 5,691,146; and 5,538,848.

More particularly, QRT-PCR or “qPCR” (Quantitative RT-PCR), which is quantitative in nature, can also be performed to provide a quantitative measure of gene expression levels. In QRT-PCR reverse transcription and PCR can be performed in two steps, or reverse transcription combined with PCR can be performed. One of these techniques, for which there are commercially available kits such as TaqMan® (Perkin Elmer, Foster City, Calif.), is performed with a transcript-specific antisense probe. This probe is specific for the PCR product (e.g. a nucleic acid fragment derived from a gene, or in this case, from a pre-miRNA) and is prepared with a quencher and fluorescent reporter probe attached to the 5′ end of the oligonucleotide. Different fluorescent markers are attached to different reporters, allowing for measurement of at least two products in one reaction.

When Taq DNA polymerase is activated, it cleaves off the fluorescent reporters of the probe bound to the template by virtue of its 5-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions can be performed in any solid support, for example, slides, microplates, 96 well plates, 384 well plates and the like so that samples derived from many individuals are processed and measured simultaneously. The TaqMan® system has the additional advantage of not requiring gel electrophoresis and allows for quantification when used with a standard curve.

A second technique useful for detecting PCR products quantitatively without is to use an intercalating dye such as the commercially available QuantiTect SYBR Green PCR (Qiagen, Valencia Calif.). RT-PCR is performed using SYBR green as a fluorescent label which is incorporated into the PCR product during the PCR stage and produces fluorescence proportional to the amount of PCR product.

Additionally, other known systems to quantitatively measure mRNA expression products include Molecular Beacons® which uses a probe having a fluorescent molecule and a quencher molecule.

In one embodiment, the polynucleotide-based detection molecules of the invention may be in the form of nucleic acid probes which can be spotted onto an array to measure RNA from the sample of a subject to be diagnosed.

As defined herein, a “nucleic acid array” refers to a plurality of nucleic acids (or “nucleic acid members”), optionally attached to a support where each of the nucleic acid members is attached to a support in a unique pre-selected and defined region. These nucleic acid sequences are used herein as detecting nucleic acid molecules.

As indicated above, assay based on micro array or RT-PCR may involve attaching or spotting of the probes in a solid support. As used herein, the terms “attaching” and “spotting” refer to a process of depositing a nucleic acid onto a substrate to form a nucleic acid array such that the nucleic acid is stably bound to the substrate via covalent bonds, hydrogen bonds or ionic interactions.

As used herein, “substrate” or “support” or “solid support”, when referring to an array, refers to a material having a rigid or semi-rigid surface. The support may be biological, non-biological, organic, inorganic, or a combination of any of these, existing as particles, strands, precipitates, gels, sheets, tubing, spheres, beads, containers, capillaries, pads, slices, films, plates, slides, chips, etc. Often, the substrate is a silicon or glass surface, (poly)tetrafluoroethylene, (poly) vinylidendifmoride, polystyrene, polycarbonate, a charged membrane, such as nylon or nitrocellulose, or combinations thereof. Preferably, at least one surface of the substrate will be substantially flat. The support may optionally contain reactive groups, including, but not limited to, carboxyl, amino, hydroxyl, thiol, and the like. In one embodiment, the support may be optically transparent.

It should be further noted that a standard Northern blot assay can also be used to ascertain an RNA transcript size and the relative amounts of the markers of the invention, or any control gene product, in accordance with conventional Northern hybridization techniques known to those persons of ordinary skill in the art.

It should be appreciated that all the detecting molecules (either nucleic acid based or amino acid based) used by the methods and kits (kits will be described herein after) of the invention are isolated and/or purified molecules. As used herein, “isolated” or “purified” when used in reference to a nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95% pure) of non-nucleotide material naturally associated with it, and thus is distinguished from isolated chromosomes. As used herein, the terms “isolated” and “purified” in the context of a proteinaceous agent (e.g., a peptide, polypeptide, protein or antibody) refer to a proteinaceous agent which is substantially free of cellular material and in some embodiments, substantially free of heterologous proteinaceous agents (i.e. contaminating proteins) from the cell or tissue source from which it is derived, or substantially free of chemical precursors or other chemicals when chemically synthesized.

As indicated above, the methods of the invention may further comprise the step of determining the catalytic activity of AChE (and optionally of BChE). Assessment of the level of catalytic activity of AChE may be performed by any method known in the art. For example, assessment of the level of catalytic activity of AChE may be carried out with analogues of acetylcholine, e.g. the acetylcholine analogue acetylthiocholine used in the “Ellman Esterase assay”.

The “Ellman Esterase assay”, described in detail by Ellman, G. L., et al. [16], is based on measurement of the change in absorbance at 405 nm. Briefly, the assay uses the thiol ester acetylthiocholine instead of the oxy ester acetylcholine. AChE hydrolyses the acetylthiocholine to produce thiocholine and acetate. The thiocholine in turn reduces the Dithiobis-Nitrobenzoic Acid (DTNB) liberating nitrobenzoate, which absorbs at 405 nm. Determining the level of catalytic activity of AChE may be also performed using indoxylacetate as an alternative substrate to the substrate used by the above Ellman Esterase assay, as described in Pohanka, M. et al. [17].

An alternative method which may be used to determine the level of AChE catalytic activity involves the natural substrate of AChE and is performed by monitoring the increase in light emission produced by the accumulation of choline in the presence of the enzyme AChE or by determining the amount of choline generated after a short interval, as describe in Birman, S. [18].

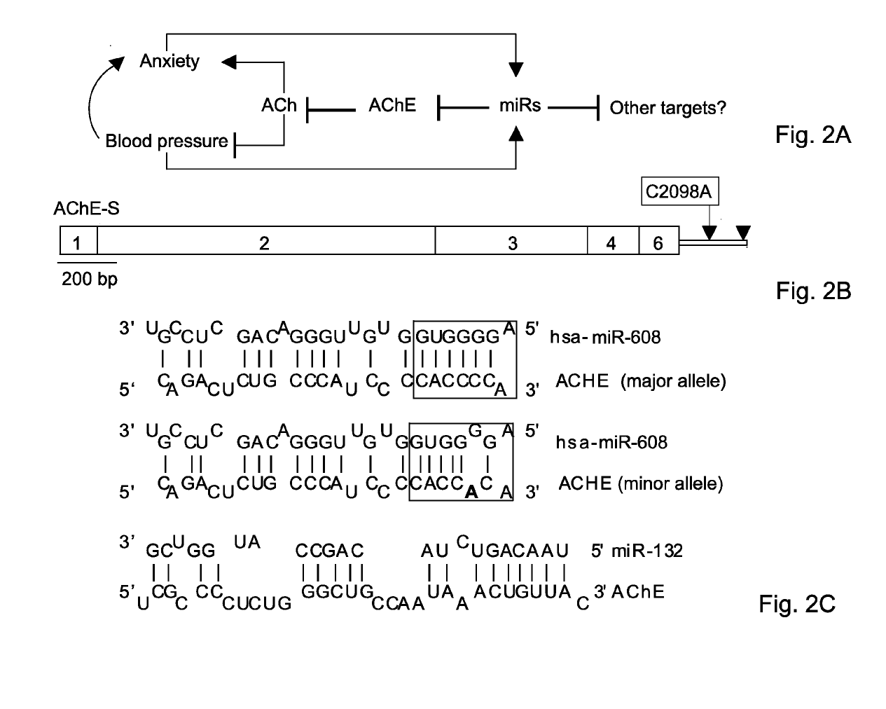

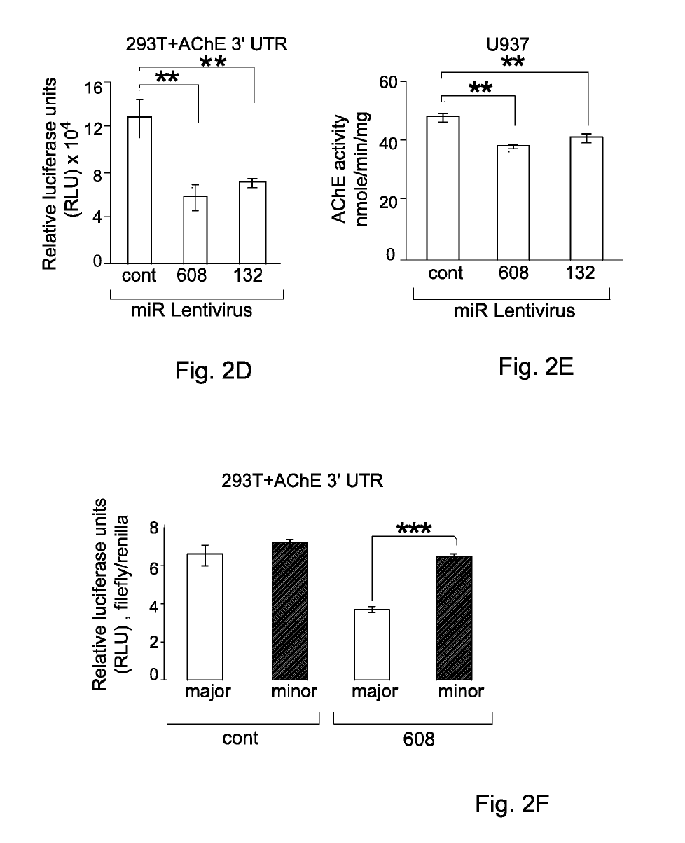

In some embodiments, and as exemplified below (e.g. the results presented in FIG. 2E), the levels of catalytic activity of AChE may be measured using an adaptation of the Ellman assay as described in Ofek et al. [19]. In brief, hydrolysis of acetylthiocholine is followed by spectrofluorometry at 405 nm, after 20 min of pre-incubation with a specific BChE inhibitor (i.e. tetra isopropyl pyrophosphoramide) to assay for AChE-specific as distinct from total cholinesterase activity.

In more specific embodiments, step (d) of the methods of the invention involves determining and calculating if the expression value obtained in step (c) is any one of, positive, negative or equal to a predetermined standard expression value (that is also referred to herein as a cutoff value) or to an expression value of said markers of the invention, specifically, at least one of AChE and at least one miR target gene (e.g., CDC42 and IL-6) in a control sample. Determination of a positive or negative expression value may be performed by comparing the expression value obtained in step (c) to a predetermined standard expression value or to an expression value of at least one of the markers of the invention in a control sample. Such a step involves calculating and measuring the difference between the expression values of the examined sample and the cutoff value and determining whether the examined sample can be defined as positive or negative. More specifically, as used herein the term “comparing” denotes any examination of the expression level and/or expression values obtained in the samples of the invention as detailed throughout in order to discover similarities or differences between at least two different samples. It should be noted that comparing according to the present invention encompasses the possibility to use a computer based approach.

It should be noted that in certain embodiments, at least one of, a positive expression or activity value of AChE and a negative expression value of at least one miR target gene, in the tested sample indicates that the subject is suffering of said at least one AChE-associated disorder. More specifically, it should be noted that in certain embodiments, the predetermined standard values (cutoff values) are calculated and obtained from populations of subjects suffering from the same AChE-associated disorder, subjects suffering from a different AChE-associated disorder, healthy subjects and subjects suffering from an unrelated disorder. Similarly, where control samples are used instead of, or in addition to predetermined cutoff values, such controls may include subjects suffering from the same AChE-associated disorder, subjects suffering from a different AChE-associated disorder, healthy subjects and subjects suffering from an unrelated disorder. Therefore, at least one of a positive expression or activity value or an equal value (or in other words, “within the range of”, when compared to cutoff representing the population of patients suffering from the same disorder) of AChE and a negative expression value of at least one miR target gene (i.e., specific embodiments for miR-608 are CDC42 and/or IL-6), reflects up-regulation of AChE expression and down regulation of the expression of said at least one miR target gene, and indicates that the examined subject belongs to a pre-established population associated with said specific AChE-associated disorder. It should be understood that such up-regulation or alternatively, down regulation, should be considered as up-regulation or down-regulation relatively to the expression/activity of healthy subjects. In more specific embodiments, such elevated expression or activity of AChE and reduced expression of at least one miRNA target gene may be expression and activity of at least one of said markers in the range of or similar to the levels of the expression in subjects suffering from the same specific AChE-associated disorder (ethnic group, age and gender matched). In contrast, at least one of a negative expression or activity value of AChE and a positive expression value of at least one miR target gene, that is a result of a proper binding of the specific miRNA to its site at AChE 3′ UTR and thereby a proper regulation of the expression of all the gene targets of said miRNA (for example, miR-608), indicates that the examined subject is healthy, or at least not suffering from the same specific AChE-associated disorder. Similarly, up-regulation of AChE followed by down regulation in the expression of other gene targets of said miRNA indicates that binding of said miRNA to its site at the AChE 3′UTR is disturbed by the presence of a particular SNP, that leads to enhanced inhibition of other targets of said miRNA. Thereby, the method of the invention provides diagnosis and prognosis of the specific AChE-associated disorder in a subject.

More specifically, a subject will be considered as suffering from a specific AChE-signaling associated condition when the expression or activity value of AChE (or as will be explained herein after, a normalized expression value of AChE) determined in a biological sample obtained from said subject is equal, within the range of or above (“positive”) a corresponding predetermined (normalized) expression/activity value of AChE obtained for a population of patients suffering from the same AChE-signaling associated disorder. Moreover, the examined subject will be considered as suffering from an AChE-disorder, when the expression value of least one miR target gene, determined in a biological sample obtained from said subject is equal, within the range of or below (“negative”) a corresponding predetermined (normalized) expression value of said at least one miR target gene obtained for a population of patients suffering from the same AChE-signaling associated disorder. In a similar manner, a subject will be considered as healthy (or at least not suffering from a specific AChE-signaling associated condition) when the expression or activity value of AChE determined in a biological sample obtained from said subject is equal, within the range of or below (“negative”) a corresponding predetermined (normalized) expression/activity value of AChE obtained for a population of healthy subjects or patients suffering from a different disorder. In certain embodiments BChE may serve as a reference control since as shown in the following Examples, the activity and expression thereof are equal in subjects displaying the minor and the major alleles.

In an alternative embodiment, when control samples are used instead of—or in addition to—a predetermined standard (cutoff), the expression value of the sample may be compared to the expression values obtained for the control sample (for example, patients suffering from the same AChE-signaling associated disorder, healthy subjects, subjects suffering from a different AChE-signaling associated disorder and subjects suffering from an unrelated disorder.

“Standard” or a “predetermined standard” as used herein, denotes either a single standard value or a plurality of standards with which the level of at least one of AChE and of at least one miR target gene expression from the tested sample is compared. The standards may be provided, for example, in the form of discrete numeric values or is calorimetric in the form of a chart with different colors or shadings for different levels of expression; or they may be provided in the form of a comparative curve prepared on the basis of such standards (standard curve). The standards may be prepared by determining the level of expression of at least one of AChE and of at least one miR target gene (CDC42 and IL-6) present in a sample obtained from a plurality of patients that were diagnosed or determined (by other means, for example by a physician, by histological techniques etc.) as suffering from a certain AChE-associated condition. The level of expression for the preparation of the standards may also be determined by various conventional methods known in the art. The methods of the invention may be carried out in parallel to a number of standards of healthy subjects and subjects of different AChE-signaling associated conditions or state and the level determined in the assayed sample is then compared to such standards. After such standards are prepared, it is possible to compare the expression level of at least one of the markers of the invention obtained from a specific tested subject to the corresponding value of the standards, and thus obtain an assaying tool. Similar approach is applied when the rate of change in the expression value is calculated in response to treatment with the therapeutic agent as will be described herein after.

Some embodiments refer to a predetermined standard expression value, or when applicable, a predetermined standard rate of change, that in other words may be defined as predetermined “cutoff” values. It should be noted that a “cutoff value”, sometimes referred to simply as “cutoff” herein, is a value that meets the requirements for both high sensitivity (true negative rate) and high specificity (true positive rate).

It should be noted that the terms “sensitivity” and “specificity” are used herein with respect to the ability of the biomarkers of the invention, at least one of AChE and of at least one miR target genes, to correctly classify a sample as belonging to a pre-established population associated with a specific condition or as will be explained herein after, a probability to respond (exhibit a beneficial effect) to a specific therapeutic agent.

According to certain and specific embodiments, the method of the invention may further comprise an additional and optional step of normalization. According to this embodiment, after determination of the expression levels of at least one of AChE and of at least one miR target gene, the expression levels of a reference control (for example, BChE, as compared to AChE, or GAPDH, when compared with miR-target genes) are also determined, and subsequently, the expression levels of at least one of AChE and of at least one miR target gene may be normalized according to the expression value of said at least one reference control/s, in the same test sample. Optionally, similar normalization is performed also relative to a control sample or a representing standard when applicable, thereby obtaining a normalized value. According to this particular embodiment, a normalized expression value of at least one of AChE and of at least one miR target gene (CDC42 and IL-6) in the test sample, and optionally relative to a control sample is obtained.

As mentioned above, the optional normalization step of the method of the invention involves normalization of the measured expression values of at least one of AChE and of at least one miR target genes, to obtain normalized expression value. In general scientific context, normalization is a process by which a measurement raw data is converted into data that may be directly compared with other so normalized data. In the context of the present invention, measurements of the expression of the marker genes used herein are prone to errors caused by, for example, unequal degradation of measured samples, different loaded quantities per assay, and other various errors. More specifically, any assayed sample may contain more or less biological material than is intended, due to human error and equipment failures. Thus, the same error or deviation applies to both the biomarker of the invention and to the control reference, whose expression is essentially constant.

Thus, division of the at least one of the marker genes raw expression value by the control reference raw expression value yields a quotient, which is essentially free from any technical failures or inaccuracies (except for major errors which destroy the sample for testing purposes) and constitutes a normalized expression value of said biomarker. This normalized expression value may then be compared with normalized cutoff values, i.e., cutoff values calculated from normalized expression values. Since control reference expression values are equal in different samples, they constitute a common reference point that is valid for such normalization.

More specifically, in the present case, the expression of control references used by the invention, i.e., BChE and/or GAPDH are equal and stable in samples displaying the minor allele of the specific examined SNP (for example C2098A) and in control samples of subjects displaying the major allele of said SNP. In certain embodiments, other control references may be used, for example, any one of actin or TAU to denote neuronal components or any other hematopoietic genes in the case of blood disorders.

It should be appreciated that similar reference to cutoff and standard values are also applicable where other parameters are being examined, for example, blood pressure, cortisol levels, etc.

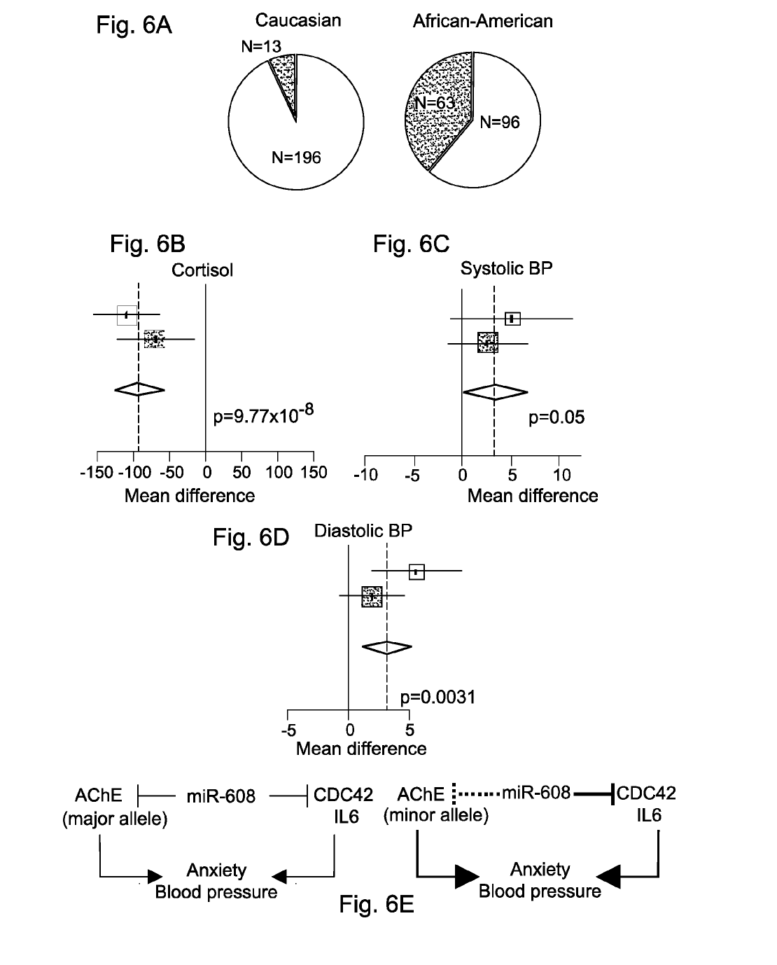

As shown in FIG. 6, when different parameters, specifically, blood pressure and cortisol levels were tested, subjects that belong to different ethnic groups show different ranges of cutoff values. Therefore, determination of the ethnic group of the examined subject may be important for accurate evaluation of the results. Thus, according to some embodiments, the methods of the invention may further comprise the step of determining the ethnic group of said subject. In more specific embodiments, the ethnic group may be any one of African, Caucasian, Ashkenazi Jews, Sepharadic Jews and Israeli Arabs.

The term Caucasian race (also Caucasoid, Europid, or Europoid) has been used herein to denote the general physical type of some or all of the populations of Europe, North Africa, the Horn of Africa, Western Asia (the Middle East), parts of Central Asia and South Asia. Historically, the term has been used to describe many peoples from these regions, without regard necessarily to skin tone. This term is often used interchangeably with “White,” although the terms are technically not synonymous.

In more specific embodiments, the invention relates to Caucasian American (CA). Caucasian Americans or White Americans are people of the United States who are considered or consider themselves White. The United States Census Bureau defines White people as those “having origins in any of the original peoples of Europe, the Middle East, or North Africa. It includes people who reported “White” or wrote in entries such as Irish, German, Italian, Lebanese, Near Easterner, Arab, or Polish.” Like all official U.S. racial categories, “White” has a “Not Hispanic or Latino” and a “Hispanic or Latino” component, the latter consisting mostly of White Mexican Americans and white Cuban Americans. More specifically, German Americans, Irish Americans, English Americans, Italian Americans, French Americans, Polish Americans, Scottish Americans, Dutch Americans, Norwegian Americans, and Swedish Americans constitute the ten largest White American ancestries.

The term African or black people is used in some socially-based systems of racial classification for humans of a dark-skinned phenotype. As a biological phenotype being “black” is often associated with the very dark skin colors of some people who are classified as “black”. But, particularly in the United States and Canada, the racial or ethnic classification also refers to people with all possible kinds of skin pigmentation from the darkest through to the very lightest skin colors, including albinos, if they are believed by others to have African ancestry, or to exhibit cultural traits associated with being “African-American”. As a result, in North America, the term “black people” is not an indicator of skin color but of socially based racial classification.

In more specific embodiments, the method of the invention is applicable for African Americans (AA) (also referred to as Black Americans or Afro-Americans, and formerly as American Negroes). These terms include citizens or residents of the United States who have total or significant partial ancestry from any of the native populations of Sub-Saharan Africa. The term is not usually used for black residents of other countries in the Americas.

Some specific embodiments the method of the invention may be based on SNPs that disturb the binding of miR-125b-3p to the 3′UTR of the AChE gene. According to these embodiments, the method of the invention comprises the steps of: First step (a), genotyping in a biological sample of said subject SNP rs17228602. In the second step (b) determining and/or identifying the presence or the absence of G2165A of SNP rs17228602 in at least one allele of the AChE gene in the genotyped sample obtained in step (a). It should be noted that the presence of at least one A allele at rs17228602 indicates that the tested subject belongs to a predetermined population associated with an AChE-signaling associated disorder.

In yet other embodiments, the method of the invention may further comprise the steps of: A third step (c) that involves determining in at least one biological sample of a subject displaying the presence of at least one of A at rs17228602, in at least one allele of the AChE gene, as identified in step (b) of the method of the invention, at least one of: (i) at least one of the level of expression and the catalytic activity of AChE in said sample, to obtain at least one of an expression value or an activity value; and (ii) the expression level of at least one gene target of miR-125b-3p in said sample to obtain an expression value of said at least one gene target;

The next step (d) involves determining at least one of: (i) if the expression or the activity value of AChE obtained in step (ci) is any one of, positive or negative with respect to a predetermined standard expression value or activity value of AChE or to an expression value or activity value of AChE in at least one control sample; and (ii) determining if the expression value of at least one gene target of miR-125b-3p obtained in step (cii) is any one of, positive or negative with respect to a predetermined standard expression value of said at least one gene target of 125b-3p or to an expression value of said target genes in at least one control sample.

It should be noted that wherein at least one of, a positive expression or activity value of said AChE and a negative expression value of at least one gene target of 125b-3p, indicates that said subject belongs to a predetermined population associated with said at least one AChE-signaling associated disorder. In certain specific embodiments, the method of the invention may further use BChE as a control marker as described above.

According to certain embodiments, the method of the invention may further comprise the steps of: determining in at least one biological sample of a subject displaying the presence of at least one of A at rs17228616, A at rs17228602, C at rs17883268 and A at rs17235010 in at least one allele of the AChE gene, as identified in step (b), at least one of blood pressure (diastolic and systolic), anxiety (state and trait), serum levels of TNF-α, serum levels of C reactive protein (CRP) and serum cortisol levels.

Still further, the method of the invention may comprise an additional step of determining the ethnic group of said subject. Such ethnic group may be any one of African, Caucasian, Ashkenazi Jews, Sepharadic Jews and Israeli Arabs.

According to certain embodiments, the methods of the invention are applicable for AChE-signaling associated disorders that are further characterized with at least one of elevated blood pressure (diastolic and systolic), elevated anxiety (trait and state), reduced cortisol levels and elevated inflammation.

In more specific embodiments these disorders may be any one of anxiety, hypertension and any immune-related disorders, specifically, inflammatory disorders and proliferative disorders. In should be understood that the methods of the invention further relates to any related conditions.

It should be noted that certain embodiments of the invention contemplate the use of different biological samples for the diagnosis and prognosis methods of the invention. The term “sample” in the present specification and claims is meant to include biological samples. Biological samples may be obtained from mammal, specifically, a human subject, include fluid, solid (e.g., stool) or tissues. The term “sample” may also include body fluids such as whole blood sample, blood cells, bone marrow, lymph fluid, serum, plasma, urine, sputum, saliva, faeces, semen, spinal fluid or CSF, the external secretions of the skin, respiratory, intestinal, and genitourinary tracts, tears, milk, any human organ or tissue, any biopsy, for example, brain, lymph node or spleen biopsies, any sample taken from any tissue or tissue extract, any sample obtained by lavage optionally of the breast ductal system, plural effusion, samples of in vitro or ex vivo cell culture and cell culture constituents. Some samples that are a priori not liquid are contacted with a liquid buffers which are then used according to the diagnostic method of the invention.

Biological samples may be obtained from all of the various families of domestic animals, as well as feral or wild animals, including, but not limited to, such animals as ungulates, bear, fish, lagamorphs, rodents, etc. Preferably, the sample is liquid, specifically, a body fluid sample, most preferably, a serum sample and of mammalian origin, specifically, human. In specific embodiment, a serum sample is being used by the invention.

Taken together, the detecting molecules used in the diagnostic and prognostic methods of the present invention (e.g., antibodies and oligonucleotide, described above) can be packaged in a diagnostic kit. Such diagnostic kits can include an antibody (e.g., labeled) and/or primers or probes of the present invention in one container and a solid phase for attaching multiple biological samples packaged in a second container as well as imaging reagent in a third container (e.g., secondary labeled antibody) with appropriate buffers and preservatives and used for diagnosis. Thus, in yet another aspect, the invention provides a kit comprising:

(a) reagents for genotyping in a biological sample of a mammalian subject at least one SNP in the 3′UTR of the AChE gene. In more specific embodiments such SNP may be at least one of rs17228616, rs17228602, rs17883268 and rs17235010.
(b) reagents for determining at least one of:
(i) at least one of the expression level or the catalytic activity of AChE in the sample; and
(ii) the expression level of at least one gene target of at least one of miR-608, miR-125b-3p, miR-4319, miR-125a-5p, miR-761, miR-214, miR-298 and miR-4283.

In some embodiments, combination of the diagnostic tools provided by the invention with standard diagnostic methods may increase sensitivity of the diagnostic methods currently known in the art. Thus, the kits of the invention may further comprise regents for determining at least one of blood pressure (diastolic and systolic), anxiety (state and trait), serum levels of TNF-α, serum levels of C reactive protein (CRP) and serum cortisol levels.

In certain embodiments, the kit of the invention may comprise at least one calibration curve. Such calibration curves may include at least one of a pre-determined calibration curve providing normalized standard expression values of the expression levels of the markers of the invention. According to other specific embodiments, the kits provided by the invention may be also designed for performing methods for assessing the state of the specific AChE disorders in a subject, or for methods of evaluating the efficacy of a given therapeutic treatment regimen or compound. These specific methods are described herein after. Accordingly, the kits of the invention may further comprise a predetermined standard [cutoff] rate of change between expression values of at least one of AChE and of at least one miR target gene (CDC42 and IL-6) obtained for at least one subject prior to said therapy and after the initiation of said therapy. In further embodiments, the kits of the invention may further comprise a predetermined calibration curve providing standard [cut-off] values of rate of change between expression values of at least one of AChE and of at least one miR target gene obtained for at least one subject in at least two temporally-separated samples obtained for said at least one subject.

Still further, in specific embodiments, where an active calibration curve is being used, the kit of the invention may further comprise any reagents necessary for preparing such calibration curve. It should be understood that such active calibration curve serves as a reference curve adjusted for each particular method used for determining the expression of at least one of AChE and of at least one miR target gene in the examined sample. In one particular example, the kit of the invention may include beads/or fixed cells stained with different fluorescent intensities representing the range of the expression levels of at least one of AChE and of at least one miR target gene in the healthy population of ethnic group, age and gender matched subjects.

According to one embodiment, the kit of the invention may be particularly applicable for use in a method for the diagnosis and prognosis of a genetic predisposition of a mammalian subject for at least one AChE-signaling associated disorder. It should be further noted that the kit of the invention may be suitable for use in any of the methods described herein.

In yet more specific embodiments, the kit of the invention may be specifically based on detecting SNPs that disturb regulation of different target genes by miR-608. Such specific kit may comprise: (a) reagents for genotyping in a biological sample of a mammalian subject SNP rs17228616. Optionally, means for determining and/or identifying the presence or the absence of C2098A of SNP rs17228616 in at least one allele of the AChE gene in the examined sample.

(b) reagents for determining at least one of: (i) at least one of the expression level or the catalytic activity of AChE in the examined sample; and (ii) the expression level of at least one gene target of miR-608.

According to specific embodiments, the target of miR-608 may be at least one of the target genes described herein before. In specific embodiments, the kit of the invention may comprise specific reagents for determining the expression level of at least one of CDC42 and IL-6.

It should be further noted that according to optional embodiments, the kit of the invention may also comprise reagents for determining the level of expression and/or the activity of BChE. As noted above, BChE may serve as a reference control.

In yet more specific embodiments, the kit of the invention may be specifically based on detecting SNPs that disturb regulation of different target genes by miR-125b-3p. Such specific kit may comprise: (a) reagents for genotyping in a biological sample of a mammalian subject SNP rs17228602. Optionally, means for determining and/or identifying the presence or the absence of G2165A of SNP rs17228602 in at least one allele of the AChE gene in the examined sample.

(b) reagents for determining at least one of: (i) at least one of the expression level or the catalytic activity of AChE in the examined sample; and (ii) the expression level of at least one gene target of miR-125b-3p (for example, miR-125b-3p that may be p53, MUC1 and TNF-α).

The kit of the invention comprises detecting molecules specific for detecting particular SNPs and for detecting the expression level of at least one of AChE and of at least one miR target gene and optionally detecting molecules specific for at least one reference control. In certain embodiments, such target genes may be any one of IL-6, CDC42, CDC44, interleukin-1 alpha (IL1A), growth hormone receptor (GHR), and TP53, that are targets of miR-608; targets of miR-125b-3p that may be p53, MUC1 and TNF-α; LIN28A, NTRK3 and CD34 that are targets of miR-125a-5p; APOBEC2, and CCDC21 that are targets of miR-761; the miR-298 target BACE1 and the targets of miR-4283 that include SIGLEC8, GNG13, and SENP1. In some specific embodiments, such detecting molecules may be selected from isolated detecting amino acid molecules and isolated detecting nucleic acid molecules.

In yet another embodiment, the kit of the invention may be adapted for examining different biological samples. In some embodiments the biological sample may be any one of a whole blood sample, blood cells, bone marrow, lymph fluid, Spleen lymph nodes tissue samples, serum, plasma, urine, sputum, saliva, faeces, semen, spinal fluid or CSF, the external secretions of the skin, respiratory, intestinal, and genitourinary tracts, tears, milk, any human organ or tissue, any sample obtained by lavage optionally of the breast ductal system, plural effusion, samples of in vitro or ex vivo cell culture and cell culture constituents.

According to specific embodiments, the biological sample may be a blood sample. The kit of the invention may therefore optionally comprise suitable mans for obtaining said sample. More specifically, for using the kit of the invention, one must first obtain samples from the tested subjects. To do so, means for obtaining such samples may be required. Such means for obtaining a sample from the mammalian subject (a) can be any means for obtaining a sample from the subject known in the art. Examples for obtaining e.g. blood or bone marrow samples are known in the art and could be any kind of finger or skin prick or lancet based device, which basically pierces the skin and results in a drop of blood being released from the skin. In addition, aspirating or biopsy needles may be also used for obtaining spleen lymph nodes tissue samples. Samples may of course be taken from any other living tissue, or body secretions comprising viable cells, such as biopsies (specifically, brain biopsies), saliva or even urine.

The present invention considers the kit of the invention in compartmental form. It should be therefore noted that the detecting molecules used for detecting the specific SNP as described herein and the expression levels of at least one of AChE and of at least one miR target gene may be provided in a kit attached to an array. As defined herein, a “detecting molecule array” refers to a plurality of detection molecules that may be nucleic acids based or protein based detecting molecules (specifically, antibodies), optionally attached to a support where each of the detecting molecules is attached to a support in a unique pre-selected and defined region.

For example, an array may contain different detecting molecules, such as specific antibodies or primers. It should be noted that each detecting molecule may be spatially arranged in a predetermined and separated location in an array. For example, an array may be a plurality of vessels (test tubes), plates, micro-wells in a micro-plate, each containing different detecting molecules, specifically, primers for detecting specific SNPs and primers, probes or antibodies specific for at least one of AChE and of at least one miR target gene (CDC42 and IL-6). An array may also be any solid support holding in distinct regions (dots, lines, columns) different and known, predetermined detecting molecules. As used herein, “solid support” is defined as any surface to which molecules may be attached through either covalent or non-covalent bonds. Different materials useful as solid support were described herein before.

It should be further appreciated that any of the reagents, substances or ingredients included in any of the methods and kits of the invention may be provided as reagents embedded, linked, connected, attached, placed or fused to any of the solid support materials described above.

Thus, according to another optional embodiment, the kit of the invention may further comprise at least one reagent for performing nucleic acid amplification based assay, such as DNA polymerase, buffer, nucleotides, PCR reaction modifiers such as polyethylene glycol (PEG), DMSO, purified water, or pre-made mixes of at least two of said PCR reagents. Such nucleic acid amplification assay may be any one of Real Time PCR, micro arrays, PCR, in situ Hybridization and Comparative Genomic Hybridization.

In other embodiments, the kits of the invention may comprise at least one reagent for any protein based assay, specifically, any immunoassay described herein before.

A further aspect of the invention relates to a method for the treatment of an AChE-signaling associated disorder in a mammalian subject. It should be noted that this therapeutic method comprises an early diagnostic step selecting the appropriate patients suitable for the specific treatment. Thus, in some embodiments, the method of the invention may comprise the steps of: In first step (a), genotyping in a biological sample of the tested subject at least one SNP in the 3′ UTR of the AChE gene. In certain embodiments, such SNP may be at least one of rs17228616, rs17228602, rs17883268 and rs17235010. In step (b) identifying and/or detecting in the genotyped sample obtained in step (a) at least one SNP that modulates the binding affinity of at least one of miR-608, miR-125b-3p, miR-4319, miR-125a-5p, miR-761, miR-214, miR-298 and miR-4283 to the 3′UTR of the AChE gene. Step (c) involves administering to a subject displaying the presence of at least one of A at rs17228616, A at rs17228602, C at rs17883268 and A at rs17235010 in at least one allele of the AChE gene, as identified in step (b), a therapeutic effective amount of at least one of: (i) at least one antagonist of at least one of miR-608, miR-125b-3p, miR-4319, miR-125a-5p, miR-761, miR-214, miR-298 and miR-4283; and (ii) at least one AChE antagonist.

It should be appreciated that any of the antagonists described in connection with the diagnostic method of the invention may also be applicable as the active ingredients in the therapeutic methods described herein. It should be further noted that certain embodiments of the invention encompass the administration of agents that modulate the expression of at least one gene target of any of the miRNAs described herein. It should be noted that the invention encompass any of the gene targets disclosed by the invention.

In more specific embodiments that relate to disorders caused by disturbed regulation of AChE and other gene targets by miR-608, the method of the invention may comprise the steps of:

In the first step (a), genotyping in a biological sample of the tested subject SNP rs17228616. The second step (b) involves determining and/or identifying the presence or the absence of C2098A of SNP rs17228616 in at least one allele of the AChE gene in the genotyped sample obtained in step (a). In third step (c) administering to a subject displaying the presence of at least one A allele at rs17228616, as identified in step (b), a therapeutic effective amount of at least one of: (i) at least one antagonist of miR-608; and (ii) at least one AChE antagonist. The invention thus provides methods for treating AChE associated disorders, specifically, anxiety, hypertension or immune-related disorders, specifically, inflammatory conditions and proliferative disorders that involve a diagnostic step for selecting the appropriate patient population for a certain appropriate treatment. The methods of the invention further involve administration of an antagonist of at least one miRNA (specifically of at least one of miR-608, miR-125b-3p, miR-4319, miR-125a-5p, miR-761, miR-214, miR-298 and miR-4283) and/or at least one AChE antagonist or an antagonist of any protein that is regulated by said miR whose binding is modified by a SNP in the AChE 3′-UTR. In certain embodiments, each of the antagonists may be either applied as the sole therapy or in combination. It should be further appreciated that any of the antagonists described herein may be further combined with any therapeutic agent in any therapeutic regimen.

The term “in combination with” such as when used in reference to a therapeutic regimen, refers to administration of two or more therapies over the course of a treatment regimen, where the therapies may be administered together or separately, and, where used in reference to drugs, may be administered in the same or different formulations, by the same or different routes, and in the same or different dosage form type.

In yet other specific embodiments that relate to disorders caused by disturbed regulation of AChE and other gene targets by miR-125b-3p, the method of the invention may comprise the steps of: First (a), genotyping in a biological sample of the subject SNP rs17228602. Second step (b) involves determining and/or identifying the presence or the absence of G2165A of SNP rs17228602 in at least one allele of the AChE gene in the genotyped sample obtained in step (a).

In step (c) administering to a subject displaying the presence of at least one A allele at rs17228602, as identified in step (b), a therapeutic effective amount of at least one of: (i) at least one antagonist of miR-125b-3p; and (ii) at least one AChE antagonist.

According to certain embodiments, the methods of the invention are applicable for treating AChE-signaling associated disorders that are further characterized with at least one of elevated blood pressure (diastolic and systolic), elevated anxiety (trait and state), reduced cortisol levels and elevated inflammation. In more specific embodiments these disorders may be any one of anxiety, hypertension and any immune-related disorders, specifically, inflammatory disorders and proliferative disorders. In should be understood that the methods of the invention further relates to any related conditions, and specifically, any disorder disclosed by the invention.

In certain and particular embodiment, the method of the invention may be specifically suitable for treating any AChE-associated disorder with the proviso that such disorder is other than hypertension. It should be appreciated that the invention further encompasses the treatment of such patients with the antagonists described above combined with any further standard treatment offered for the specific disease. In non-limiting examples, proliferative disorders that are associated with AChE-signaling may be treated with the conventional chemotherapeutic or immunotherapeutic agents in combination with the active ingredients suggested by the invention, as disclosed herein. For inflammatory disorders associated with AChE-signaling, the present invention offers combination of the agents of the invention as described herein (e.g., antagonist of the different miRNAs and/or AChE antagonist/s) with conventional anti-inflammatory agents.

The term “treatment or prevention” as used herein, refers to the complete range of therapeutically positive effects of administrating to a subject including inhibition, reduction of, alleviation of, and relief from, an AChE-signaling associated condition and illness, symptoms associated with AChE-signaling disorder or undesired side effects of such disorders. More specifically, treatment or prevention of progression or deterioration of the disease in response to a treatment with a non-effective, or deleterious therapeutic agent, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms.

The therapeutic agents described by the invention, the different miRNA and/or AChE antagonists can be administered to the individual per se or as part of a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism. As used herein, the term “active ingredient” refers to the agent, specifically, any miRNA or AChE antagonist described herein before, for example, polynucleotide and/or expression vector as described by the present invention accountable for the intended biological effect. Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases. Herein, the term “excipient” refers to an inert substance added to a pharmaceutical composition to further facilitate administration of an active ingredient.

Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference. Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intracardiac, intranasal, or intraocular injections. Alternately, one may administer the pharmaceutical composition in a local rather than systemic manner, for example, via injection of the pharmaceutical composition directly into a tissue region of a patient.

Pharmaceutical compositions that may be used by the present invention may be manufactured by processes well known in the art, e.g., by means of conventional mixing, dissolving, granulating, dragee-making, levigating, emulsifying, encapsulating, entrapping, or lyophilizing processes. Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen. For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art. For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofiuoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

The pharmaceutical composition of the present invention may also be formulated in rectal compositions such as suppositories or retention enemas, using, for example, conventional suppository bases such as cocoa butter or other glycerides. Pharmaceutical compositions suitable for use in the context of the present invention include compositions wherein the active ingredients are contained in an amount effective to achieve the intended purpose. More specifically, a “therapeutically effective amount” means an amount of active ingredients (e.g., the agent, specifically, any of the antagonists described by the invention) effective to prevent, alleviate, or ameliorate symptoms of the pathology or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of which follows.

As shown by the examples, subjects displaying SNP rs17228616 that disturbs binding of miR-608 to the 3′UTR of AChE, are characterized with reduced cortisol levels (FIG. 6) and elevated blood pressure, that are clear markers of anxiety. Since at least one target of the miR-608, specifically, CDC42 is also involved in anxiety, measuring and monitoring the levels of this marker, or any gene target of miR-608, may provide sensitive evaluation and assessment of the status and condition of the patient, level of risk etc. In certain embodiments, such evaluation may provide an early and active (real-time) assessment of the patient even before the appearance of any symptoms of the disease.

Thus, in yet another aspect the invention provides a method for assessing the anxiety level of a mammalian subject. In certain embodiments the method of the invention may comprise the steps of: In step (a) genotyping in a biological sample of said subject SNP rs17228616. In step (b) determining and/or identifying the presence or the absence of C2098A of SNP rs17228616 in at least one allele of the AChE gene in the genotyped sample of step (a). Step (c) involves determining in at least one biological sample of a subject displaying the presence of A at rs17228616 in at least one allele of the AChE gene, as identified or detected in step (b), at least one of: (i) at least one of the level of expression and the catalytic activity of AChE in the sample, to obtain at least one of an expression value or an activity value; and (ii) determining the expression level of at least one of miR-608 target genes, for example, any one of CDC42, IL-6, CDC44, IL1A, GHR and TP53, in said sample to obtain an expression value of at least one of these target genes.

The next step (d) involves determining at least one of: (i) if the expression or the activity value of AChE obtained in step (ci) is any one of, positive or negative with respect to a predetermined standard expression value or activity value of AChE, or to an expression value or activity value of AChE in at least one control sample; and (ii) determining if the expression value of at least one of the miR-608 target genes obtained in step (cii) is any one of, positive or negative with respect to a predetermined standard expression value of said at least one of CDC42 and IL-6 or to the expression value of said at least one of the miR-608 target genes in at least one control sample; It should be noted that wherein at least one of a positive expression or activity value of said AChE and a negative expression value of at least one of the miR-608 target genes, specifically, CDC42 and IL-6 indicates that said subject belongs to a predetermined population associated with a specific level of anxiety.

Similar approach may be applied and adapted for assessing the level of an inflammatory condition or of any of the AChE-associated disorders described herein, when applicable.

It should be further noted that in addition to assessment of the disease state or condition, the methods of the invention may further provide a tool for monitoring and early prognosis of disease deterioration. According to such methods, the detection step may be performed using several samples obtained from the patients in different time intervals (temporally separated samples). In certain embodiments, such patient is treated with a therapeutic agent and therefore, the method further provides a tool for assessing efficacy of such treatment.

Thus, a further aspect of the invention relates to a method for assessing the efficacy of a treatment of a mammalian subject suffering from at least one AChE-signaling associated disorder, with a therapeutic agent and monitoring the disease progression. More specifically, the method of the invention may comprise the steps of: In step (a) genotyping in a biological sample of said subject at least one of the following SNPs, rs17228616, rs17228602, rs17883268, rs17228609 and rs17235010. In step (b) determining and/or identifying the presence or the absence of at least one of A at rs17228616, A at rs17228602, C at rs17883268, A at rs17228609 and A at rs17235010 in at least one allele of the AChE gene in the genotyped sample of step (a).

In step (c) determining in at least one biological sample of a subject displaying the presence of A at rs17228616 in at least one allele of the AChE gene, as identified or detected in step (b), at least one of: (i) at least one of the level of expression and the catalytic activity of AChE in the sample, to obtain at least one of an expression value or an activity value; and (ii) determining the expression level of at least one target gene of any of the miRNAs described herein in the sample to obtain an expression value of at least one of said target genes in the sample;

In step (d) repeating step (c) to obtain expression values of at least one of AChE and any of the miR-target genes described by the invention, for example, CDC42 and IL-6, for at least one more temporally-separated test sample. It should be notes that at least one of such temporally separated sample is obtained after the initiation of the treatment. Step (e) involves calculating the rate of change of the expression values of at least one of AChE and miR-target genes between the temporally-separated test samples. Step (f) involves determining if the rate of change obtained in step (e) is positive or negative with respect to a predetermined standard rate of change determined between at least two temporally separated samples or to the rate of change calculated for expression values in at least one control sample obtained from at least two temporally separated samples. It should be noted that at least one sample of said at least two samples is obtained after the initiation of the treatment.

In more specific embodiments, where at least one of a negative rate of change in the expression value of AChE and a positive rate of change at least one of in the expression value of at least one of the miR-target genes, indicates that the subject exhibits a beneficial response to the specific treatment; thereby monitoring the efficacy of a treatment with a therapeutic agent and the disease progression.

In certain embodiments, the invention provides specific methods for monitoring the disease progression and assessing efficacy of treatment of AChE-associated disorders displaying SNP rs17228616 that disturbs binding of miR-608 to AChE 3′UTR. According to some specific embodiments, miR-608 target genes that may be used as suitable markers may include any of the miR-608 target genes and specifically, any one of CDC42 and IL-6.

In certain embodiments, the methods of the invention may further comprise the steps of: determining in at least one biological sample of a subject displaying the presence of at least one of A at rs17228616, A at rs17228602, C at rs17883268, A at rs17228609 and A at rs17235010 in at least one allele of the AChE gene, as identified in step (b), at least one of blood pressure (diastolic and systolic), anxiety (state and trait), serum levels of TNF-α, serum levels of C reactive protein (CRP) and serum cortisol levels.

The method of the invention provides determining the suitability for treatment of a patient suffering from a disease associated with AChE-signaling a-priori, i.e., before the onset of such treatment, or in most cases, in early stages of the treatment, enabling a personalized treatment. Thus, the methods of the invention will enable avoiding a treatment that will potentially aggravate the AChE-signaling associated condition in a given patient and provides the selection of an appropriate treatment that will be beneficial to the specific patient.

Similarly, the method of the invention provides determining the suitability and efficacy of treatment of a patient suffering from an AChE-signaling associated condition during said treatment, i.e. after the onset thereof, to monitor the effect of the treatment on the patient. Wherein, by using the method of the invention, the expression value of at least one miR target gene, decline below a predetermined value or below the expression value in a control sample obtained from the patient prior to the onset of the treatment, or during the treatment, the treatment may be ceased, and alternative treatments may be sought for by the method of the invention, thus avoiding the deleterious effect that may accompany ensuing such treatment on said patient.

The method of the invention provides the use of the specific SNPs, AChE and at least one gene target of at least one miRNA, as biomarkers (optionally combined biomarkers) for sensing the effect of a therapeutic agent, on a patient and thereby determining the efficacy of a suggested treatment on a particular patient.

As mentioned above, final step (f) involves determination if the rate of change obtained for the temporally-separated samples, is any one of, positive, negative or equal to a predetermined standard rate of change (cutoff value). More specifically, such standard rate of change is determined prior and after the initiation of a specific treatment with the same therapeutic agent, for a population of subjects suffering from the same pathologic disorder defined as “responders”. It should be also understood that in certain embodiments, the level of at least one of AChE and of at least one miR target gene (CDC42 and IL-6, that are specific and non-limiting examples for miR-608 target genes) in a sample after treatment, may also be evaluated as compared to standard value obtained for a population of ethnic group, age and gender matched healthy subject that do not suffer from any pathologic disorder. In an alternative embodiment, where control samples are used, this step involves determining if the rate of change is positive, negative or equal as compared to the rate of change calculated for expression values in at least one control sample obtained prior and following the specific treatment. Such step involves calculating and measuring the difference between the rate of change in the expression values of the examined sample and the standard rate of change (cutoff value) and determining whether the examined sample can be defined as positive or negative. It must be understood that the standard rate of change (cutoff value) were calculated for populations of patients suffering from the same pathologic disorder (that are not treated), subjects treated with the same therapeutic agent (responders or non-responders) and healthy subjects (ethnic group, age and gender matched). It should be also appreciated that the predetermined standard values (cutoff) may be presented in a standard curve. Similarly, if control samples are used in the method of the invention, such controls may include samples from patients suffering from the same pathologic disorder (untreated or treated with the specific therapeutic agent), subjects suffering from the same condition treated with the same therapeutic agent that are responders, samples of non-responders and samples of healthy subjects (ethnic group, age and gender matched).

According to certain embodiments, a positive rate of change of at least one miR target gene (CDC42 and IL-6) expression value reflects reduced suppression of the specific miRNA (miR-608, in case of CADC42 and IL-6) in response to said treatment relative to prior sample and or cut offs as above and therefore indicates that the examined subject responds to the certain treatment and specifically exhibits a beneficial response to the treatment. More particularly, a positive rate of change indicates that the examined subject belongs to a pre-established population associated with a higher probability to exhibit a beneficial response to the specific examined treatment. A negative rate of change in the expression value relative to prior sample and or cut offs as above indicates that the examined subject does not respond and specifically, does not exhibit a beneficial response to the treatment. In more particular embodiments, a negative rate of change reflects reduction of at least one miR target gene (CDC42 and IL-6) expression in response to treatment with a specific therapeutic compound revealing a pathologic state in the patient and thereby indicating that the specific therapeutic agent is not appropriate for said examined patient. The efficacy of the specific treatment in a specific subject is thereby determined.

In sensing the specific effect of a particular therapeutic agent on a certain subject, the method of the invention may be used for personalized medicine, namely adjusting and customizing healthcare with decisions and practices being suitable to the individual patient by use of any additional information collected at different stages of the disease.

A higher expression value of at least one miR target gene in response to a certain treatment (up-regulated expression) is determined as “positive” rate of change and a lower expression value of at least one miR target gene in response to said treatment (down-regulated expression) is determined as “negative” rate of change. As noted herein, before calculating rates of change in at least one of AChE and of at least one miR target gene expression in response to treatment, the rate of change should be compared to the standard rates obtained for populations of subjects suffering from the same condition untreated or treated with the same compound that are defined as responders or non-responders, as well as for healthy control subjects.

To disambiguate, a “positive”, “higher” or “up-regulated” expression value of at least one miR target gene, or rate of change in the expression of at least one miR target gene or a “negative”, “lower” or “down-regulated” expression/activity value of AChE in response to treatment determined in the biological sample indicates that the subject is suitable for the specific treatment and exhibits a beneficial response. In other embodiments, the specific therapeutic agent is suitable and effective to the particular examined patient, where the expression value (specifically, the normalized expression values or rate of change) of at least one of AChE and of at least one miR target gene determined in the biological sample is in the range of the normalized expression value of at least one of AChE and of at least one miR target gene obtained in a suitable control sample or a corresponding predetermined standard expression value (cutoff values) of at least one of AChE and of at least one miR target gene.

As shown by the present invention as disclosed in the following Examples, in case of SNPs that reduced, or lower expression of at least one miR target gene (for example the gene targets of miR0-608, CDC42 and IL-6) and optionally, increased or higher expression/activity level of AChE, is correlated with elevated blood pressure and reduced cortisol levels, and thereby indicates that the tested subject suffers from an AChE-associated disorder. Thus, “Low level of expression”, or “negative” expression values or rate of change, when applicable, as used herein for at least one miR target gene (CDC42 and IL-6, denotes a level significantly (e.g. as determined by statistical determination) lower than a predetermined standard.

On the other hand, increased, or higher expression of at least one miR target gene (CDC42 and IL-6) and optionally, reduced or lower expression/activity level of AChE, in response to treatment with a therapeutic agent is correlated with an improvement in the status of the specific an AChE-associated disorder, and thereby indicates that the tested subject would respond and exhibit a beneficial response to treatment with the specific therapeutic agent. Thus, “high level of expression”, or “positive” expression values, or rate of change, when applicable, as used herein for at least one miR target gene, denotes a level significantly (e.g. as determined by statistical determination) higher than the expression before treatment that is in the range of a predetermined standard. It should be noted that such standard value is predetermined for a population of patients suffering from the same pathologic condition that perform beneficial effect “responders”. Therefore, expression value or rate of change that is within the range of such cutoff indicates that the examined subject belongs to a population of responders. A particular example for such SNP may be rs17228616 that disturbs binding of miR-608 to AChE 3′UTR, and thereby the regulation of other gene targets of said miR, for example, CDC42 and IL-6.

However, it should be understood that the invention further encompasses the option of SNPs that enhance or tightens binding or interaction of a specific miR to the AChE 3′UTR and thereby lead to reduction in AChE expression (activity). Moreover, the tight enhanced binding to AChE 3′UTR weakens the suppression of other targets of such miR that results in elevation of the expression level of such other targets of the same miR. Thus, “high level of expression”, or “positive” expression values or rate of change, when applicable, as used herein for at least one miR target gene my be correlated with non-responsiveness or disease progression, while reduced, or lower expression of at least one miR target gene and optionally, elevated or higher expression/activity level of AChE, in response to treatment with a therapeutic agent is correlated with an improvement in the status of the specific an AChE-associated disorder.

Cutoff values may be used as a control sample, said cutoff values being the result of a statistical analysis of at least one of AChE and of at least one miR target gene (CDC42 and IL-6) expression value differences in pre-established populations with either an AChE associated condition, reflecting “non-responsiveness” or deleterious effects of the specific therapeutic agent or alternatively, populations of “responsive” or “healthy” subjects with no symptoms of an AChE-associated disorder in response to a successful treatment.

It should be emphasized that the nature of the invention is such that the accumulation of further patient and/or healthy donors data may improve the accuracy of any obtained cutoff values, which are usually based on an ROC (Receiver Operating Characteristic) curve generated according to accumulated patient and/or healthy donors data using, for example, a commercially available analytical software program. The at least one of AChE and of at least one miR target gene expression values are selected along the ROC curve for optimal combination of sensitivity and specificity, which are as close to 100% as possible, and the resulting values are used as the cutoff values that distinguish between patients who will display an AChE-associated condition or symptoms thereof at a certain rate in response to a specific treatment, and those who will display a beneficial response to the same therapeutic agent that is reflected by reduced symptoms (with said given sensitivity and specificity), for example, reduced blood pressure and increased cortisol levels. The ROC curve may evolve as more and more patient and healthy donor data and related at least one of AChE and of at least one miR target gene expression values are recorded and taken into consideration, modifying the optimal cutoff values and improving sensitivity and specificity. Thus, any cutoff values should be viewed as a starting point that may shift as more patient-AChE disorder data in response to a specific treatment allows more accurate cutoff value calculation. It should be noted that healthy subjects used in the invention as control samples or standard curves are ethnic group, age and gender matched subjects. It should be further appreciated that standard curves for “responders” may be prepared specifically for each pathologic condition, and in some embodiments, also for each therapeutic agent (or treatment regimen).

In a specific example of SNP rs17228616 that disturbs the interaction of miR-608 to AChE 3′UTR, elevation of AChE levels/activity and reduced expression of other targets of said miR, correlate with an AChE disorder. Moreover, a “positive” rate of change of at least one miR target gene (CDC42 and IL-6) and a “negative” rate of change in AChE expression values in response to a certain treatment as specified above indicates that the specific examined treatment is effective and results in a beneficial response in the tested subject. A “negative” rate of change in at least one miR target gene (CDC42 and IL-6) and a “positive” rate of change in AChE expression/activity value indicate that said treatment is not effective. The method of the invention thereby provides monitoring the efficacy of a treatment with a therapeutic agent and the disease progression.

It should be noted that the term “response”, “responsiveness”, “responsive” or “responder” to treatment with a specific therapeutic agent refers to an improvement in at least one relevant clinical parameter as compared to an untreated subject diagnosed with the same pathology (e.g., the same type, stage, degree and/or classification of the anxiety or inflammatory condition), or as compared to the clinical parameters of the same subject prior to said treatment.

The term “non-responder” or “non-responsive” to treatment using a specific therapeutic agent, refers to a patient not experiencing an improvement in at least one of the clinical parameter and is diagnosed with the same condition as an untreated subject diagnosed with the same pathology (e.g., the same type, stage, degree and/or classification of the chronic inflammatory condition), or experiencing the clinical parameters of the same subject prior to such treatment.

Of course, more samples taken in more time-points may provide a statistically robust analysis of said expression trends, and may also be utilized as a method for continuous monitoring of subjects, especially those still undergoing and those that have undergone therapy. The more samples are available over a given time period, the higher is the resolution of the expression patterns of least one of AChE and of at least one miR target gene (CDC42 and IL-6) during said period.

Interestingly, an example for the human variability in this pathway is presented in a previous publication of the inventors [19]. This publication shows that human volunteers subjected to injection of interleukin-6 showed either enhancement or suppression of AChE expression. Without being bound by any theory, it is likely that at least some of the SNPs disclosed in the present invention Moreover, patients showing enhanced AChE, showed faster regain of white blood cell counts when compared to patients displaying reduced levels of AChE, following this experimental inflammation-causing insult.

It should be understood that the number of samples collected and used for evaluation of the subject may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. Furthermore, to assess the trend in expression rates according to the invention, it is understood that the rate of change may be calculated as an average rate of change over at least three samples taken in different time points, or the rate may be calculated for every two samples collected at adjacent time points. It should be appreciated that the sample may be obtained from the monitored patient in the indicated time intervals for a period of several months or several years. More specifically, for a period of 1 year, for a period of 2 years, for a period of 3 years, for a period of 4 years, for a period of 5 years, for a period of 6 years, for a period of 7 years, for a period of 8 years, for a period of 9 years, for a period of 10 years, for a period of 11 years, for a period of 12 years, for a period of 13 years, for a period of 14 years, for a period of 15 years or more.

In practice, for monitoring purpose, to detect a decline or elevation in least one of AChE and of at least one miR target gene expression, at least two “temporally-separated” test samples must be collected from the treated patient, and preferably more. The expression of at least one of AChE and of at least one miR target gene is then determined using the method of the invention, applied for each sample. The rate of change in this biomarker expression is then calculated by determining the difference in expression values (specifically, normalized values) of said least one of AChE and of at least one miR target gene between any two samples obtained from the same patient in different time-points or time intervals. This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. It should be noted that such interval could be as indicated herein above.

When calculating the rate of change, one may use any two samples collected at different time points from the patient. To ensure more reliable results and reduce statistical deviations to a minimum, averaging the calculated rates of several sample pairs is preferable.

As indicated above, in order to execute the diagnostic and prognostic methods of the invention, at least two different samples, and preferably, more than two, must be obtained, from the subject in order to calculate the rate of change in the expression of at least one of AChE and of at least one miR target gene (herein defined as the markers or biomarkers of the invention) in response to treatment with a specific compound. By obtaining at least two and preferably more biological samples from a subject and analyzing them according to the methods of the invention, may be effective for predicting, monitoring and early diagnosing molecular alterations indicating an AChE-associated condition, such as anxiety, hypertension or inflammation state in said patient in response to a specific treatment. Thus, the prognostic method of the invention may be applicable for early, sub-symptomatic diagnosis of an AChE-associated condition in a treated subject, when used for analysis of more than a single sample along the time-course of diagnosis, treatment and follow-up. An “early diagnosis” provides diagnosis prior to appearance of clinical symptoms. Prior as used herein is meant days, weeks, months or even years before the appearance of such symptoms. More specifically, at least 1 week, at least 1 month, 2 months, 3 months, 4 months, 5 months, 6 months, 7 months, 8 months, 9 months, 10 months, 11 months, 12 months, or even few years before clinical symptoms appear.

Thus, the invention therefore further provides a prognostic method. “Prognosis” is defined as a forecast of the future course of a disease or disorder, based on medical knowledge. This highlights the major advantage of the instant invention over prior art, namely, the ability to predict an AChE-associated condition, such as anxiety, immune-related disorder or hypertension indicating regression or deterioration of the condition in patients treated with a non-appropriate therapeutic agent. This early prognosis facilitates the selection of appropriate treatment regimens that may minimize the regression or deterioration of the condition, individually to each patient, as part of personalized medicine.

As disclosed herein above, the invention thus provides a method for predicting the efficacy and suitability of a specific treatment. As used herein the phrase “predicting or evaluating efficacy of a treatment” refers to determining the likelihood that a specific treatment using a therapeutic agent is efficient or non-efficient in treating the AChE-associated condition, e.g., the success or failure of the treatment in treating the AChE-associated condition in a subject in need thereof.

The term “efficacy” as used herein refers to the extent to which the treatment with a specific therapeutic compound, for example, an antagonist of at least one miRNA (specifically of at least one of miR-608, miR-125b-3p, miR-4319, miR-125a-5p, miR-761, miR-214, miR-298 and miR-4283) and/or at least one AChE antagonist or alternatively, any standard therapy, produces a beneficial result, e.g., an improvement in one or more symptoms of the pathology (caused by the AChE-associated condition) and/or clinical parameters related to the pathology as described herein below.

According to some embodiments of the invention, a treatment with a therapeutic agent that either directly or indirectly affects an AChE-associated disorder, specifically, anxiety, immune-related disorder, hypertension or inflammation, is considered efficient in treating said condition if it exerts an improvement in at least one relevant clinical parameter related to said condition in the treated subject as compared to an untreated subject diagnosed with the same condition, or as compared to the clinical parameters related to the said condition of the same subject prior to said treatment.

The present invention relates to the diagnosis and monitoring and treatment of subjects or patients, in need thereof. By “patient” or “subject in need” it is meant any organism who may be affected by the above-mentioned conditions, and to whom the monitoring, diagnosis and therapeutic methods herein described are desired, including humans, domestic and non-domestic mammals such as canine and feline subjects, bovine, simian, equine and murine subjects, rodents, domestic birds, aquaculture, fish and exotic aquarium fish. It should be appreciated that the diagnosed or monitored subject may be also any reptile or zoo animal. More specifically, the methods of the invention are intended for mammals. By “mammalian subject” is meant any mammal for which the proposed therapy is desired, including human, livestock, equine, canine, and feline subjects, most specifically humans.

The diagnostic kits and methods of the invention further provide a tool for a “tailor-made” or personalized therapy, by identifying subjects suffering from a specific AChE-associated disorder that are likely to be benefit from treatment with a specific therapeutic agent.

Thus, according to a further aspect of some embodiments of the present invention there is provided a method of selecting a treatment regimen for treating a subject diagnosed with an AChE-signaling associated condition according to the invention, the method comprising: (a) determining and evaluating the efficacy of a treatment with a therapeutic agent given to a subject suffering from said condition according to the method of some embodiments of the invention, and (b) selecting a treatment regimen based on the evaluation; thereby selecting the treatment regimen for treating the subject diagnosed with said condition.

The following Tables 1 and 2, provide information regarding different SNPs encompassed by the present invention.

TABLE 1
SNPs of AChE-S (NM_000665.3)

| | | | | | | | Amino |
|---|---|---|---|---|---|---|---|
| Chr. | mRNA | dbSNP rs# | | dbSNP | Protein | Codon | acid |
| position | pos | cluster id | Function | allele | residue | pos | pos |
| 100487889 | 1930 | rs147386700 | synonymous | T | His [H] | 3 | 597 |
| | | | contig | C | His [H] | 3 | 597 |
| | | | reference | | | | |
| 100489989 | 1658 | rs11554090 | synonymous | T | Leu [L] | 1 | 507 |
| | | | contig | C | Leu [L] | 1 | 507 |
| | | | reference | | | | |
| 100490015 | 1632 | rs139429533 | missense | T | Val [V] | 2 | 498 |
| | | | contig | C | Ala [A] | 2 | 498 |
| | | | reference | | | | |
| 100490059 | 1588 | rs145048252 | synonymous | A | Glu [E] | 3 | 483 |
| | | | contig | G | Glu [E] | 3 | 483 |
| | | | reference | | | | |
| 100490074 | 1573 | rs151107784 | synonymous | T | His [H] | 3 | 478 |
| | | | contig | C | His [H] | 3 | 478 |
| | | | reference | | | | |
| 100490077 | 1570 | rs7636 | synonymous | T | Pro [P] | 3 | 477 |
| | | | contig | C | Pro [P] | 3 | 477 |
| | | | reference | | | | |
| 100490080 | 1567 | rs149813374 | synonymous | C | Val [V | 3 | 476 |
| | | | contig | G | Val [V] | 3 | 476 |
| | | | reference | | | | |
| 100490093 | 1554 | rs145811992 | missense | C | Pro [P] | 2 | 472 |
| | | | contig | T | Leu [L] | 2 | 472 |
| | | | reference | | | | |
| 100490131 | 1516 | rs113533105 | synonymous | T | Tyr [Y] | 3 | 459 |
| | | | contig | C | Tyr [Y] | 3 | 459 |
| | | | reference | | | | |
| 100490136 | 1511 | rs148991713 | missense | A | Thr [T] | 1 | 458 |
| | | | contig | G | Ala [A] | 1 | 458 |
| | | | reference | | | | |
| 100490151 | 1496 | rs3028261 | frame shift | | | 1 | 453 |
| | | | frame shift | GG | Gly [G] | 1 | 453 |
| | | | contig | G | Gly [G] | 1 | 453 |
| | | | reference | | | | |
| 100490152 | 1495 | rs145683970 | synonymous | A | Gln [Q] | 3 | 452 |
| | | | contig | G | Gln [Q] | 3 | 452 |
| | | | reference | | | | |
| 100490171 | 1476 | rs61729577 | missense | G | Gly [G] | 2 | 446 |
| | | | contig | C | Ala [A] | 2 | 446 |
| | | | reference | | | | |
| 100490242 | 1405 | rs74418820 | synonymous | A | Pro [P] | 3 | 422 |
| | | | contig | G | Pro [P] | 3 | 422 |
| | | | reference | | | | |
| 100490322 | 1325 | rs41281001 | missense | T | Phe [F] | 1 | 396 |
| | | | contig | G | Val [V] | 1 | 396 |
| | | | reference | | | | |
| 100490407 | 1240 | rs138349745 | synonymous | A | Ser [S] | 3 | 367 |
| | | | contig | G | Ser [S] | 3 | 367 |
| | | | reference | | | | |
| 100490809 | 1184 | rs116131706 | missense | A | Thr [T] | 1 | 349 |
| | | | contig | G | Ala [A] | 1 | 349 |
| | | | reference | | | | |
| 100490810 | 1183 | rs144995873 | synonymous | T | Asn [N] | 3 | 348 |
| | | | contig | C | Asn [N] | 3 | 348 |
| | | | reference | | | | |
| 100490822 | 1171 | rs17880119 | synonymous | A | Glu [E] | 3 | 344 |
| | | | contig | G | Glu [E] | | 344 |
| | | | reference | | | | |
| 100490823 | 1170 | rs17228588 | missense | G | Gly [G] | 2 | 344 |
| | | | contig | A | Glu [E] | 2 | 344 |
| | | | reference | | | | |
| 100490856 | 1137 | rs8286 | missense | A | Glu [E] | 2 | 333 |
| | | | contig | T | Val [V] | 2 | 333 |
| | | | reference | | | | |
| 100490936 | 1057 | rs141785210 | synonymous | G | Thr [T] | 3 | 306 |
| | | | contig | A | Thr [T] | 3 | 306 |
| | | | reference | | | | |
| 100490980 | 1013 | rs150598944 | missense | A | Ser [S] | 1 | 292 |
| | | | contig | G | Gly [G] | 1 | 292 |
| | | | reference | | | | |
| 100490981 | 1012 | rs17881163 | synonymous | T | Gly [G] | 3 | 291 |
| | | | contig | C | Gly [G] | 3 | 291 |
| | | | reference | | | | |
| 100490982 | 1011 | rs142818130 | missense | A | Asp [D] | 2 | 291 |
| | | | contig | G | Gly [G] | 2 | 291 |
| | | | reference | | | | |
| 100491014 | 979 | rs147370685 | synonymous | A | Thr [T] | 3 | 280 |
| | | | contig | G | Thr [T] | 3 | 280 |
| | | | reference | | | | |
| 100491047 | 946 | rs17228581 | synonymous | A | Thr [T] | 3 | 269 |
| | | | contig | G | Thr [T] | 3 | 269 |
| | | | reference | | | | |
| 100491092 | 900 | rs144986926 | missense | G | Arg [R] | 2 | 254 |
| | | | contig | A | His [H] | 2 | 254 |
| | | | reference | | | | |
| 100491114 | 879 | rs143875983 | missense | T | Leu [L] | 2 | 247 |
| | | | contig | C | Pro [P] | 2 | 247 |
| | | | reference | | | | |
| 100491149 | 844 | rs113406515 | synonymous | A | Ala [A] | 3 | 235 |
| | | | contig | G | Ala [A] | 3 | 235 |
| | | | reference | | | | |
| 100491152 | 841 | rs142452543 | synonymous | T | Ser [S] | 3 | 234 |
| | | | contig | C | Ser [S] | 3 | 234 |
| | | | reference | | | | |
| 100491167 | 826 | rs151335006 | synonymous | A | Thr [T] | 3 | 229 |
| | | | contig | G | Thr [T] | 3 | 229 |
| | | | reference | | | | |
| 100491179 | 814 | rs139672629 | synonymous | A | Pro [P] | 3 | 225 |
| | | | contig | G | Pro [P] | 3 | 225 |
| | | | reference | | | | |
| 100491361 | 632 | rs144426398 | missense | A | Asn [N] | 1 | 165 |
| | | | contig | G | Asp [D] | 1 | 165 |
| | | | reference | | | | |
| 100491406 | 587 | rs77109413 | missense | C | His [H] | 1 | 150 |
| | | | contig | T | Tyr [Y] | 1 | 150 |
| | | | reference | | | | |
| 100491415 | 578 | rs146564868 | missense | A | Ile [I] | 1 | 147 |
| | | | contig | G | Val [V] | 1 | 147 |
| | | | reference | | | | |
| 100491441 | 552 | rs141299237 | missense | T | Leu [L] | 2 | 138 |
| | | | contig | G | Arg [R] | 2 | 138 |
| | | | reference | | | | |
| 100491451 | 542 | rs17885778 | missense | G | Ala [A] | 1 | 135 |
| | | | contig | C | Pro [P] | 1 | 135 |
| | | | reference | | | | |
| 100491491 | 502 | rs145703834 | synonymous | C | Arg [R] | 3 | 121 |
| | | | contig | T | Arg [R] | 3 | 121 |
| | | | reference | | | | |
| 100491520 | 473 | rs61729575 | missense | C | Gln [Q] | 1 | 112 |
| | | | contig | G | Glu [E] | 1 | 112 |
| | | | reference | | | | |
| 100491550 | 443 | rs1056867 | nonsense | T | xxx [X] | 1 | 102 |
| | | | contig | C | Gln [Q] | 1 | 102 |
| | | | reference | | | | |
| 100491588 | 405 | rs114367422 | missense | A | Glu [E] | 2 | 89 |
| | | | contig | G | Gly [G] | 2 | 89 |
| | | | reference | | | | |
| 100491637 | 356 | rs143172740 | missense | G | Val [V] | 1 | 73 |
| | | | contig | A | Met [M] | 1 | 73 |
| | | | reference | | | | |
| 100491671 | 322 | rs17228574 | synonymous | C | Ser [S] | 3 | 61 |
| | | | contig | T | Ser [S] | 3 | 61 |
| | | | reference | | | | |
| 100491685 | 308 | rs17234982 | missense | A | Arg [R] | 1 | 57 |
| | | | contig | G | Gly [G] | 1 | 57 |
| | | | reference | | | | |
| 100491687 | 306 | rs114782198 | missense | T | Leu [L] | 2 | 56 |
| | | | contig | C | Pro [P] | 2 | 56 |
| | | | reference | | | | |
| 100491729 | 264 | rs116298479 | missense | T | Met [M] | 2 | 42 |
| | | | contig | C | Thr [T] | 2 | 42 |
| | | | reference | | | | |
| 100491742 | 251 | rs13246682 | missense | A | Lys [K] | 1 | 38 |
| | | | contig | G | Glu [E] | 1 | 38 |
| | | | reference | | | | |
| 100491753 | 240 | rs17881553 | missense | A | Gln [Q] | 2 | 34 |
| | | | contig | G | Arg [R] | 2 | 34 |
| | | | reference | | | | |

TABLE 2
SNPs of AChE-R (NM_015831.2)

| | | | | | | | Amino |
|---|---|---|---|---|---|---|---|
| Chr. | mRNA | dbSNP rs# | | dbSNP | Protein | Codon | acid |
| position | pos | cluster id | Function | allele | residue | pos | pos |
| 100488638 | 1934 | rs17886728 | missense | A | Thr [T] | 1 | 599 |
| | | | contig | C | Pro [P] | 1 | 599 |
| | | | reference | | | | |
| 100488658 | 1914 | rs1799806 | missense | G | Arg [R] | 2 | 592 |
| | | | contig | C | Pro [P] | 2 | 592 |
| | | | reference | | | | |
| 100489989 | 1658 | rs11554090 | synonymous | T | Leu [L] | 1 | 507 |
| | | | contig | C | Leu [L] | 1 | 507 |
| | | | reference | | | | |
| 100490015 | 1632 | rs139429533 | missense | T | Val [V] | 2 | 498 |
| | | | contig | C | Ala [A] | 2 | 498 |
| | | | reference | | | | |
| 100490059 | 1588 | rs145048252 | synonymous | A | Glu [E] | 3 | 483 |
| | | | contig | G | Glu [E] | 3 | 483 |
| | | | reference | | | | |
| 100490074 | 1573 | rs151107784 | synonymous | T | His [H] | 3 | 478 |
| | | | contig | C | His [H] | 3 | 478 |
| | | | reference | | | | |
| 100490077 | 1570 | rs7636 | synonymous | T | Pro [P] | 3 | 477 |
| | | | contig | C | Pro [P] | 3 | 477 |
| | | | reference | | | | |
| 100490080 | 1567 | rs149813374 | synonymous | C | Val [V] | 3 | 476 |
| | | | contig | G | Val [V] | 3 | 476 |
| | | | reference | | | | |
| 100490093 | 1554 | rs14581192 | missense | C | Pro [V] | 2 | 472 |
| | | | contig | T | Leu [L] | 2 | 472 |
| | | | reference | | | | |
| 100490131 | 1516 | rs113533105 | synonymous | T | Tyr [Y] | 3 | 459 |
| | | | contig | C | Tyr [Y] | 3 | 459 |
| | | | reference | | | | |
| 100490136 | 1511 | rs148991713 | missense | A | Thr [T] | 1 | 458 |
| | | | contig | G | Ala [A] | 1 | 458 |
| | | | reference | | | | |
| 100490151 | 1496 | rs3028261 | frame shift | | | 1 | 453 |
| | | | frame shift | GG | Gly [G] | 1 | 453 |
| | | | contig | G | Gly [G] | 1 | 453 |
| | | | reference | | | | |
| 100490152 | 1495 | rs145683970 | synonymous | A | Gln [Q] | 3 | 452 |
| | | | contig | G | Gln [Q] | 3 | 452 |
| | | | reference | | | | |
| 100490171 | 1476 | rs61729577 | missense | G | Gly [G] | 2 | 446 |
| | | | contig | C | Ala [A] | 2 | 446 |
| | | | reference | | | | |
| 100490242 | 1405 | rs74418820 | synonymous | A | Pro [P] | 3 | 422 |
| | | | contig | G | Pro [P] | 3 | 422 |
| | | | reference | | | | |
| 100490322 | 1325 | rs41281001 | missense | T | Phe [F] | 1 | 396 |
| | | | contig | G | Val [V] | 1 | 396 |
| | | | reference | | | | |
| 100490407 | 1240 | rs138349745 | synonymous | A | Ser [S] | 3 | 367 |
| | | | contig | G | Ser [S] | 3 | 367 |
| | | | reference | | | | |
| 100490809 | 1184 | rs116131706 | missense | A | Thr [T] | 1 | 349 |
| | | | contig | G | Ala [A] | 1 | 349 |
| | | | reference | | | | |
| 100490810 | 1183 | rs144995873 | synonymous | T | Asn [N] | 3 | 348 |
| | | | contig | C | Asn [N] | 3 | 348 |
| | | | reference | | | | |
| 100490822 | 1171 | rs17880119 | synonymous | A | Glu [E] | 3 | 344 |
| | | | contig | G | Glu [E] | 3 | 344 |
| | | | reference | | | | |
| 100490823 | 1170 | rs17228588 | missense | G | Gly [G] | 2 | 344 |
| | | | contig | A | Glu [E] | 2 | 344 |
| | | | reference | | | | |
| 100490856 | 1137 | rs8286 | missense | A | Glu [E] | 2 | 333 |
| | | | contig | T | Val [V] | 2 | 333 |
| | | | reference | | | | |
| 100490936 | 1057 | rs141785210 | synonymous | G | Thr [T] | 3 | 306 |
| | | | contig | A | Thr [T] | 3 | 306 |
| | | | reference | | | | |
| 100490980 | 1013 | rs150598944 | missense | A | Ser [S] | 1 | 292 |
| | | | contig | G | Gly [G] | 1 | 292 |
| | | | reference | | | | |
| 100490981 | 1012 | rs17881163 | synonymous | T | Gly [G] | 3 | 291 |
| | | | contig | C | Gly [G] | 3 | 291 |
| | | | reference | | | | |
| 100490982 | 1011 | rs142818130 | missense | A | Asp [D] | 2 | 291 |
| | | | contig | G | Gly [G] | 2 | 291 |
| | | | reference | | | | |
| 100491014 | 979 | rs147370685 | synonymous | A | Thr [T] | 3 | 280 |
| | | | contig | G | Thr [T] | 3 | 280 |
| | | | reference | | | | |
| 100491047 | 946 | rs17228581 | synonymous | A | Thr [T] | 3 | 269 |
| | | | contig | G | Thr [T] | 3 | 269 |
| | | | reference | | | | |
| 100491093 | 900 | rs144986926 | missense | G | Arg [R] | 2 | 254 |
| | | | contig | A | His [H] | 2 | 254 |
| | | | reference | | | | |
| 100491114 | 879 | rs143875983 | missense | T | Leu [L] | 2 | 247 |
| | | | contig | C | Pro [P] | 2 | 247 |
| | | | reference | | | | |
| 100491149 | 844 | rs113406515 | synonymous | A | Ala [A] | 3 | 235 |
| | | | contig | G | Ala [A] | 3 | 235 |
| | | | reference | | | | |
| 100491152 | 841 | rs142452543 | synonymous | T | Ser [S] | 3 | 234 |
| | | | contig | C | Ser [S] | 3 | 234 |
| | | | reference | | | | |
| 100491167 | 826 | rs151335006 | synonymous | A | Thr [T] | 3 | 229 |
| | | | contig | G | Thr [T] | 3 | 229 |
| | | | reference | | | | |
| 100491179 | 814 | rs139672629 | synonymous | A | Pro [P] | 3 | 225 |
| | | | contig | G | Pro [P] | 3 | 225 |
| | | | reference | | | | |
| 100491361 | 632 | rs144426398 | missense | A | Asn [N] | 1 | 165 |
| | | | contig | G | Asp [D] | 1 | 165 |
| | | | reference | | | | |
| 100491406 | 587 | rs77109413 | missense | C | His [H] | 1 | 150 |
| | | | contig | T | Tyr [Y] | 1 | 150 |
| | | | reference | | | | |
| 100491415 | 578 | rs146564868 | missense | A | Ile [I] | 1 | 147 |
| | | | contig | G | Val [V] | 1 | 147 |
| | | | reference | | | | |
| 100491441 | 552 | rs141299237 | missense | T | Leu [L] | 2 | 138 |
| | | | contig | G | Arg [R] | 2 | 138 |
| | | | reference | | | | |
| 100491451 | 542 | rs17885778 | missense | G | Ala [A] | 1 | 135 |
| | | | contig | C | Pro [P] | 1 | 135 |
| | | | reference | | | | |
| 100491491 | 502 | rs145703834 | synonymous | C | Arg [R] | 3 | 121 |
| | | | contig | T | Arg [R] | 3 | 121 |
| | | | reference | | | | |
| 100491520 | 473 | rs61729575 | missense | C | Gln [Q] | 1 | 112 |
| | | | contig | G | Glu [E] | 1 | 112 |
| | | | reference | | | | |
| 100491550 | 443 | rs1056867 | nonsense | T | xxx[X] | 1 | 102 |
| | | | contig | C | Gln [Q] | 1 | 102 |
| | | | reference | | | | |
| 100491588 | 405 | rs114367422 | missense | A | Glu [E] | 2 | 89 |
| | | | contig | G | Gly [G] | 2 | 89 |
| | | | reference | | | | |
| 100491637 | 356 | rs143172740 | missense | G | Val [V] | 1 | 73 |
| | | | contig | A | Met [M] | 1 | 73 |
| | | | reference | | | | |
| 100491671 | 322 | rs17228574 | synonymous | C | Ser [S] | 3 | 61 |
| | | | contig | T | Ser [S] | 3 | 61 |
| | | | reference | | | | |
| 100491685 | 308 | rs17234982 | missense | A | Arg [R] | 1 | 57 |
| | | | contig | G | Gly [G] | 1 | 57 |
| | | | reference | | | | |
| 100491687 | 306 | rs114782198 | missense | T | Leu [L] | 2 | 56 |
| | | | contig | C | Pro [P] | 2 | 56 |
| | | | reference | | | | |
| 100491729 | 264 | rs116298479 | missense | T | Met [M] | 2 | 42 |
| | | | contig | C | Thr [T] | 2 | 42 |
| | | | reference | | | | |
| 100491742 | 251 | rs13246682 | missense | A | Lys [K] | 1 | 38 |
| | | | contig | G | Glu [E] | 1 | 38 |
| | | | reference | | | | |
| 100491753 | 240 | rs17881553 | missense | A | Gln [Q] | 2 | 34 |
| | | | contig | G | Arg [R] | 2 | 34 |
| | | | reference | | | | |

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Unless defined otherwise, all technical and scientific terms used herein have the same meaning as commonly understood by one of ordinary skill in the art to which this invention is related. The following terms are defined for purposes of the invention as described herein.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook & Russell, 2001.

### ABBREVIATIONS

### Acetylcholine—ACh or Ach

### Acetylcholinesterase—AchE or ACHE

Single nucleotide polymorphism(s)—SNP(s)
Micro-ribonucleic acid—miRNA or miR

### Dulbecco's Modified Eagle Medium—DMEM

### Polyethylenimine—PEI

Phosphate buffered saline—PBS
Fetal bovine serum—FBS

### Butylcholinesterase—BChE

3′ terminus untranslated-region—3′ UTR
Surface plasmon resonance—SPR

### Streptavidin—SA

Polymerase chain reaction—PCR

### Health, Risk Factors, Exercise Training, and Genetics—HERITAGE

### Experimental Procedures

### AChE SNP Localization

Acetylcholinesterase (AChE) single nucleotide polymorphisms (SNPs) were described in Hasin et al. [20] and in the NCBI dbSNP database (www.ncbi.nlm.nih.gov/snp), and were co-localized with predicted miR binding sites to AChE according to Hanin and Soreq 2011 [21]. The primate specificity of this miR was determined using miRbase (www.mirbase.org).

### Lentivirus Preparation

miRNA expression vectors containing overexpression cassettes for pre-miR-132, -608, -125b or a scrambled sequence for control, were obtained from GeneCopoeia (MD, USA). 1 μg of packaging, 0.7 μg of envelope plasmid, and 1 μg miR vector were added to 250 μl of serum-free Dulbecco's Modified Eagle Medium (DMEM) supplemented with 1 mM glutamine and 50 mg/ml gentamycin. 10 μl of 1 mg/ml polyethylenimine (PEI) (Sigma, Israel) was added to the mix, which was then vortexed and incubated for 10 min at room temperature. HEK-293T cells, grown to 90% confluence, were washed with PBS and 5 ml of serum-free medium was added. Cells were then transduced with the PEI/plasmid mix. After 4 hours of incubation, 0.5 ml of fetal bovine serum (FBS) was added. Virus-containing medium was collected 24 and 48 hours later, filtered through 0.45 mm-pore filters, and stored in aliquots at −80° C.

### Cell Lines

Cells were grown in a humidified atmosphere at 37° C., 5% CO₂. U937 cells were grown in RPMI-1640 (Sigma-Aldrich) and HEK-293T cells were grown in DMEM. All media was supplemented with 10% FBS, 2 mM L-glutamine, 1,000 units/ml penicillin, 0.1 mg/ml streptomycin sulfate, and 0.25 microgram/ml amphotericin B. All reagents except RPMI were obtained from Biological Industries (Beit Haemek, Israel).

### Cholinesterase Activity

The levels of catalytic activity of AChE in human serum and brain samples and in U937 cells (assayed 96 hours post-lentiviral infection) were measured using an adaptation of the Ellman assay as described in Ofek et al. [19]. In brief, hydrolysis of acetylthiocholine (ATCh, Sigma) was followed by spectrofluorometry (GENios Pro, Tecan, Maennedorf, Switzerland) at 405 nm, after 20 mM of pre-incubation with 5×10⁻⁵ M of a specific BChE inhibitor (tetra isopropyl pyrophosphoramide, Sigma) to assay for AChE-specific as distinct from total cholinesterase activity. (N=3/treatment).

### Luciferase and Life-Death Assay

The AChE 3′UTR sequence was cloned into the MicroRNA Target Selection System plasmid (System Biosciences, CA, USA), a dual reporter system containing Firefly luciferase and a cytotoxic sensor, in which cells survive only if tested miRs bind the 3′UTR. HEK-293T cells transfected with miRNA Target Selection-AChE 3′UTR were selected for 3 weeks using 5 μg/ml puromycin. Stable cells were then infected with lentiviruses encoding miR-132, -125b, -608, or control sequence. 72 hours post-infection cytotoxic drug was added according to manufacturer's instructions. Cell survival was determined 8 days post-infection. Luciferase activities were measured 72 hours post-infection using the DualLuciferase® Reporter Assay System (Promega, WI, USA) according to manufacturer's instructions.

### Site-Directed Mutagenesis and Luciferase Assays

The C2098A SNP AChE 3′UTR sequence (also referred to herein as C2851A or rs17228616) (in pUC57) was constructed by mutagenesis of the major allele of the AChE 3′UTR, using the Quickchange II protocol (Stratagene, CA, USA) with the forward primer CCATCCCCACCACACCCCGACGTCCCC (as denoted by SEQ ID NO. 1) and reverse primer GGGGACGTCGGGGTGTGGTGGGGATGG (as denoted by SEQ ID NO. 2). Major or minor allele fragments were then cloned into the psiCHECK-2 vector (Promega) using the XhoI and NotI restriction enzymes. Luciferase activities were measured 72 hours post-infection by DualLuciferase® Reporter Assay System as above.

### MicroRNA-Target Predicted Structure and Binding Energy

miR-target binding energy and structure were predicted using the RNAhybrid algorithm (http://bibiserv.techfak.uni-bielefeld.de/rnahybrid/).

### Surface Plasmon Resonance (SPR)

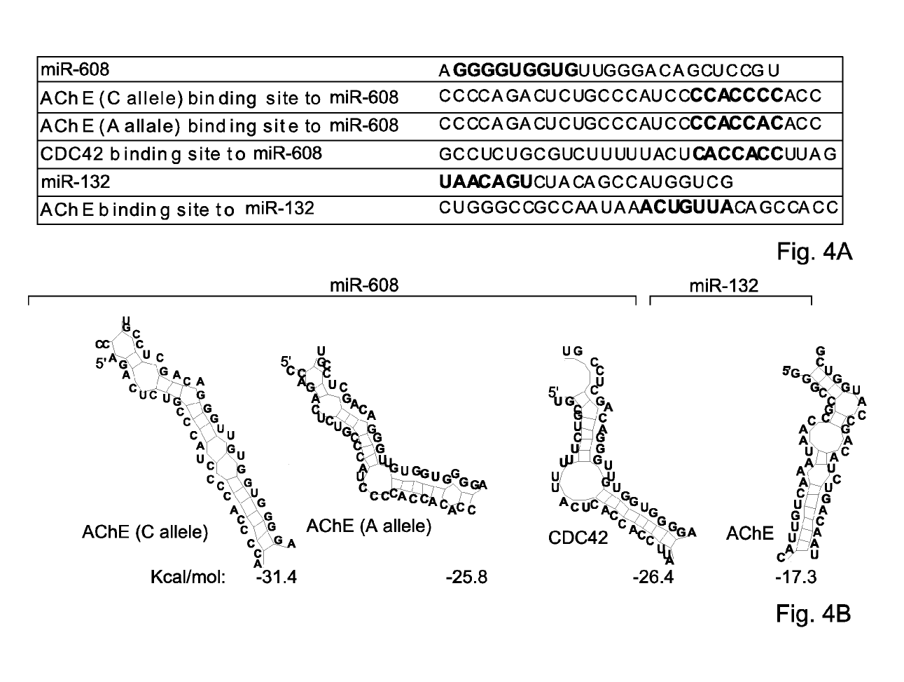

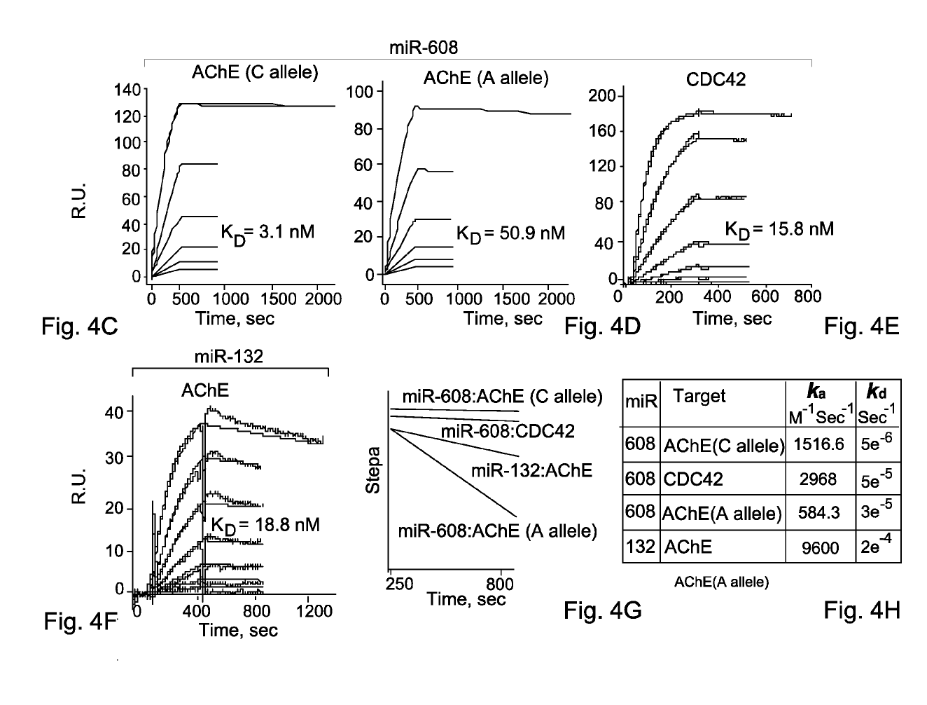

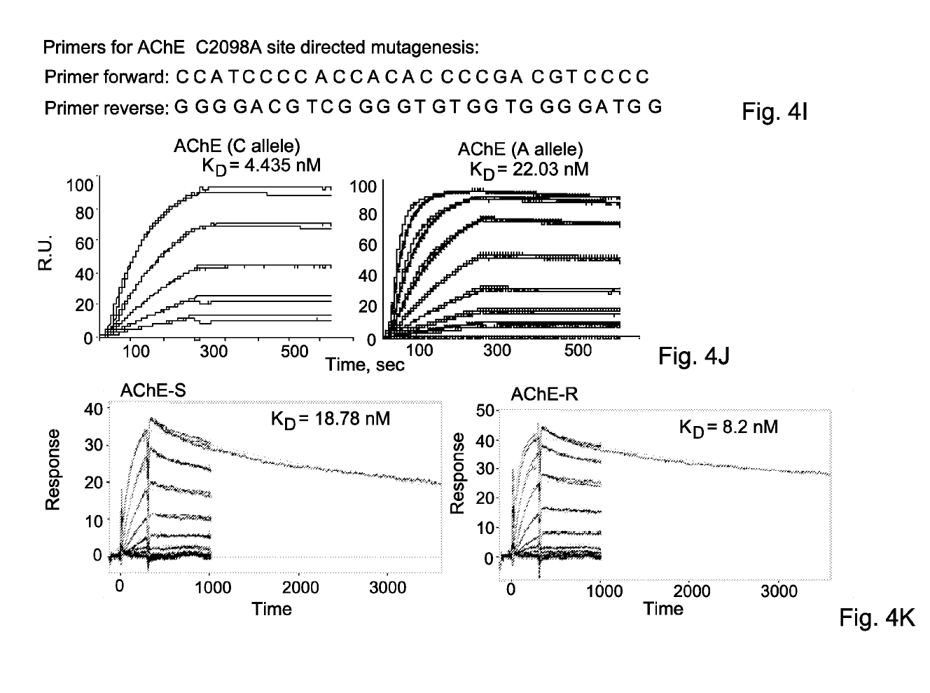

SPR biosensor experiments for characterization of the interactions between microRNAs and their mRNA targets were conducted using a Biacore 3000 instrument docked with research-grade streptavidin (SA) chips (Biacore AB, Uppsala, Sweden). Oligonucleotides were synthesized as RNA oligos and were fully 2′O-methylated for protection. Oligos representing miR binding sites on target mRNAs were 5′ biotinylated for immobilization to the SA chips whereas oligos representing miR sequence were not biotinylated (Syntezza-IDT, Israel). All sequences appear in FIG. 4. Standard buffer HBS (10 mM HEPES, 150 mM NaCl, 3.4 mM EDTA, 0.005% surfactant P20, pH 7.4) was used for the analyses, all of which were carried out at 25° C. Biotinylated oligonucleotides were dissolved in 100% 1,1,1,3,3,3-hexafluoro-2-propanol (HFIP) to 1 mM, diluted (1:5000) into 10 mM sodium acetate pH 5.0, and injected at 10 μL/min. The levels of C-allele AChE and A-allele AChE, CDC42, and AChE-miR-132 binding site captured on the chip were 325, 305, 296, and 113 RU, respectively. The surface was stabilized with a continuous flow of HBS buffer at a rate of 10 μL/min. The miR-608 or miR-132 oligos were diluted in buffer (serial two-fold dilutions, 0.3125, 0.625, 1.25, 2.5, 5, and 10 μM) and injected over the flow cells for 2 min at 10 μL/min, with 5-min association & 5-min dissociation, except for the highest concentration that was allowed to dissociate for 1 hr. The sensograms were double-referenced (responses were corrected with both blank buffer injections and the response from the reference flow cell) and were fit using a mathematical model of a simple 1:1 interaction (Scrubber 2 software). All experiments were run in duplicates.

### Immunoblots

Cells and brain samples were lysed using a solution containing 0.01 M Tris HCl pH=7.4, 1 M NaCl, 1 mM EGTA, and 1% TX-100. SDS-PAGE separation and transfer to nitrocellulose followed standard procedures. Proteins were visualized using primary antibodies against CDC42 (ab64533, Abcam, MA, USA, 1:1000), IL6 (ab6672, Abcam, 1:1000), and GAPDH for normalization (2118, Cell Signaling, MA, USA, 1:2000), followed by horseradish peroxidase-conjugated goat anti rabbit antibodies (Jackson Laboratories, PA, USA, 1:10,000) and enhanced chemiluminescence (EZ-EC1, Biological Industries, Beit-Haemek, Israel).

### Human Brain Samples

Postmortem cortical samples of apparently healthy aged volunteers were obtained from The Netherlands Brain Bank (NBB, Netherlands Institute for Neuroscience, Amsterdam). All material was collected from donors for or from whom a written informed consent for brain autopsy and the use of the material and clinical information for research purposes had been obtained by the NBB.

### Human Brain Tissue Genotyping

DNA was extracted using Direct PCR reagent (Viagen Biotech, CA, USA) supplemented with 0.3 mg/ml proteinase K (Roche, USA) according to the manufacturer's instructions. Genotyping of the A-allele of rs17228616 (C2098A) versus the C-allele was performed using TaqMan genotyping primers and AccuStart genotyping ToughMix low ROX (Quanta BioSciences, MD, USA). Due to the location of the SNP in a GC-rich sequence the TaqMan assay distinguishes between the prevalent (CC) and SNP (AA and CA) alleles. To differentiate further between homozygous (AA) and heterozygous (CA) rs17228616, sequencing of PCR-amplified DNA was performed.

### MicroRNA Quantification

MicroRNA levels were determined using the TaqMan MicroRNA Assay system (Applied Biosystems, CA, USA) according to the manufacturer's instructions, and quantified using the comparative (delta delta) Ct method with normalization to snoRNA135.

### HERITAGE Family Study Cohort

### HERITAGE Sample Description

The Health, Risk Factors, Exercise Training, and Genetics (HERITAGE) Family Study was designed to investigate the role of genetic variation on the response to regular exercise of multiple risk factors and cardio respiratory fitness. A total of 461 individuals (198 men and 263 women) from 150 two-generation families of African-American (172) or Caucasian (289) origin with complete data were available for this study.

### Serum Analyses

Blood samples were collected in the morning after a 12-hour fast and serum was separated by centrifugation at 2,000 g (15 min at 4° C.). Serum aliquots were stored at −80° C. until use.

### HERITAGE Sample Genotyping

For DNA analysis purposes, the inventors used DNA from previously screened individuals [8] for whom DNA, stress and inflammation data was available. Genomic DNA was prepared from lymphoblastoid cell lines generated from HERITAGE samples. DNA genotyping was performed by the SNaPshot™ method (Applied Biosystems) and by sequencing. Sequencing of PCR-amplified DNA was performed, using the primers Fwd GTTCGACCACTACAGCAAGC (as denoted by SEQ ID NO. 17), and Rev CCCGCTCCTGCATAGACTC (as denoted by SEQ ID NO. 18).

### Statistics

P values for the difference between the genotypes of the subjects were calculated using the likelihood ratio test. The P value was the exact conditional tail probability given the marginal, as was assessed by 100,000 Monte Carlo simulations. Multiple regression analysis was performed using R statistical software. Other analysis was done using R software, including meta-analysis of both populations of the cohort: African-Americans and Caucasians. Meta-analysis was performed using the “Meta” package, with fixed effects and continuous outcome data. Inverse variance weighting was used for pooling. The DerSimonian-Laird estimate for the between-study variance was used in the random effects model by default. Statistical significance was calculated using Student's t-test or by one- or two-way ANOVA with LSD post-hoc, where appropriate. Unless otherwise noted, ±SEM is shown for all graphs.

Determining the Level of Factors Associated with Inflammation and of Factors Associated with Anxiety

Measures of state and trait anxiety were obtained on a subset of the families as part of the study questionnaire. Cortisol and Estradiol (E₂) were assayed directly using a commercially available kit (Diagnostic Systems Laboratories Inc., Webster, Tex.). Plasma C-reactive protein (CRP) at baseline was measured with a high-sensitivity solid-phase chemiluminescent immunometric assay (IMMULITE 2000 High Sensitivity CRP, Diagnostic Products Corporation, Los Angeles, Calif.) implemented on an automated immunoassay instrument (Diagnostic Products Corporation, Los Angeles, Calif.). In a sample of 48 blind duplicates, the intra-class correlation was 0.98 and the coefficient of variation was 6.4%. TNF-α levels were determined in duplicate, using commercially available enzyme linked immunosorbent assay (ELISA) kits (Quantikine; R&D Systems; Minneapolis, Minn.) according to the manufacturer's protocol. Each plate included a standard curve and known positive and negative controls. Absorbance (A) was read against the blank at a wavelength of 450 nm using a microtiter ELISA reader. Intra- and inter-assay coefficients of variation were less than 10% for all analyses. The assay sensitivity was 0.5˜5.5 pg/ml. Blood-pressure measures were taken using the Colin STBP-780 automated BP unit (San Antonio, Tex.). This system employs two electrocondenser microphones embedded in the cuff. The sound signal is synchronized to the electrocardiograph (ECG) R wave, and a detection algorithm is used to determine both SBP and DBP. Earphones worn by the technician allowed for a manual confirmation of the algorithm-determined BP values. In addition, the instrument's printer function enabled the technician to visually confirm the accuracy of the reading with its graphic representation of the Korotkoff sounds (K-sounds).

### Example 1

### Clustered miR-Silencing Mutations in the 3′-UTR of the AChE Gene

Previous studies suggested a genetic predisposition for acute stress reactions in certain individual carriers of acetylcholinesterase (AChE) polymorphisms [1, 22, 23], placing them at higher risk of developing post-traumatic stress disorder (PTSD) and inflammatory diseases.

The identification of patients with genetic polymorphisms that affect the expression of serum enzymes involved in regulating the levels of ACh, including AChE and BChE, may hence identify patients with a higher tendency to develop such diseases. Established polymorphisms that directly affect activity levels include ‘atypical’ BChE, which bears the D70G mutation that confers acute sensitivity to anti-AChEs [24, 25], and the BChE K variant, which increases the risk of drug irresponsive Alzheimer's disease[26].

However, single nucleotide polymorphism (SNP) silencers of miR interactions were not yet reported in this gene family. Statistically significant prevalence of certain known or previously undetected SNPs in the tested populations may enable those and the new 3′-UTR SNPs to serve as markers for the future development of PTSD and inflammatory diseases in a tested population, and may allow to identify new therapeutic targets.

Therefore, the 3′-untranslated region (UTR) of the AChE gene of healthy young volunteers was analyzed, were a cluster of SNPs was identified, several of which may interrupt miR functions. The cluster of putative miR-silencing SNPs in the 3′-UTR of the human AChE gene is schematically shown in FIG. 1. FIG. 1A shows a schematic structure of the human AChE gene and FIGS. 1B and 1C show schematic structures of its AChE-S (having the accession number NM_{—}000665.3) and AChE-R (having the accession number NM_{—}015831.2) transcripts, respectively, as well as the site location of the mapped SNPs (indicated by numbers).

Interestingly, five of the SNPs located in the 3′-UTR region may potentially silence at least one miR, namely the SNPs referred to in FIG. 1D, which are shown with their complementary regions in the respective miR. Presented in FIG. 1D are ACHE G2165A (SNP rs17228602, included in the corresponding binding sites for miR-4319, miR-125a-5p and miR-125b-3p, having the nucleic acid sequence denoted by SEQ ID NOs. 9, 10, and 11, respectively. The location of said SNP is also indicated in FIG. 1D by the number 11), ACHE G2071A (SNP rs17235010, included in the corresponding binding sites for miR-4283, having the nucleic acid sequence denoted by SEQ ID NO. 12. The location of said SNP is also indicated in FIG. 1D by the number 14, and also indicated herein as G2042A), ACHE T2402C (SNP rs17883268, included in the corresponding binding sites for miR-761, miR-214 and miR-298 having the nucleic acid sequence denoted by SEQ ID NO. 13, 14 and 15, respectively. The location of said SNP is also indicated in FIG. 1D by the number 13) and ACHE C2098A (SNP rs17228616, included in the corresponding binding sites for miR-608, having the nucleic acid sequence denoted by SEQ ID NO. 16. The location of said SNP is also indicated in FIG. 1D by the number 15 and also denoted herein as C2851A). As also shown in FIG. 1, SNP G2165A and SNP T2402C are located in the 3′ region of AChE-R, while SNP G2071A and SNP C2098A are located in the 3′ region of AChE-S. It is noted that of the 8 sequences shown below, three (namely the binding site of miR-4319, miR-4283 as well as the binding site of miR-607) are human-specific, one (namely the binding site of miR-298) which also exists in mice but is not conserved and four evolutionarily conserved SNPs (namely the binding site of miR-125a-5p, miR-125b-3p, miR-761 and the binding site of miR-214). It is noted that none of the SNPs were found in the miR-132 binding site.

### Example 2

### The Primate-Specific miR-608 Potently Targets AChE

One of the many currently known SNPs on the AChE gene [20] is the 3′-UTR C2098A substitution, also known as ACHE C2851A (rs17228616, www.ncbi.nlm.nih.gov/projects/SNP). The C2098A substitution is located in the seed region of a putative miR-608 binding site, as schematically presented in FIG. 2C and in FIG. 1D (the nucleic acid sequence of AChE major allele in the putative miR-608 binding site is denoted by SEQ ID NO. 52), where this SNP is indicated as “ACHE minor allele” (included in the putative miR-608 binding site as denoted by SEQ ID NO. 16) [21]. The C2098A substitution is also located close to the binding site of the AChE-targeting miR-132 (see FIG. 2B and FIG. 2C). Interestingly, this C-to-A mismatch in the 3′-untranslated region of the AChE-S transcript of rs17228616 SNP in the AChE gene has been reported to appear in about 20% of the US population, but no function had been yet reported for it.

To validate this predicted miR-target interaction, AChE 3′UTR was cloned into a MicroRNA Target Selection vector carrying an upstream cytotoxic sensor and firefly luciferase. Human embryonic kidney 293T (HEK-293T) cells were stably-transfected with the above plasmid carrying the AChE 3′UTR, and then infected with miR-expressing lentiviruses, as described under the Experimental procedure section above.

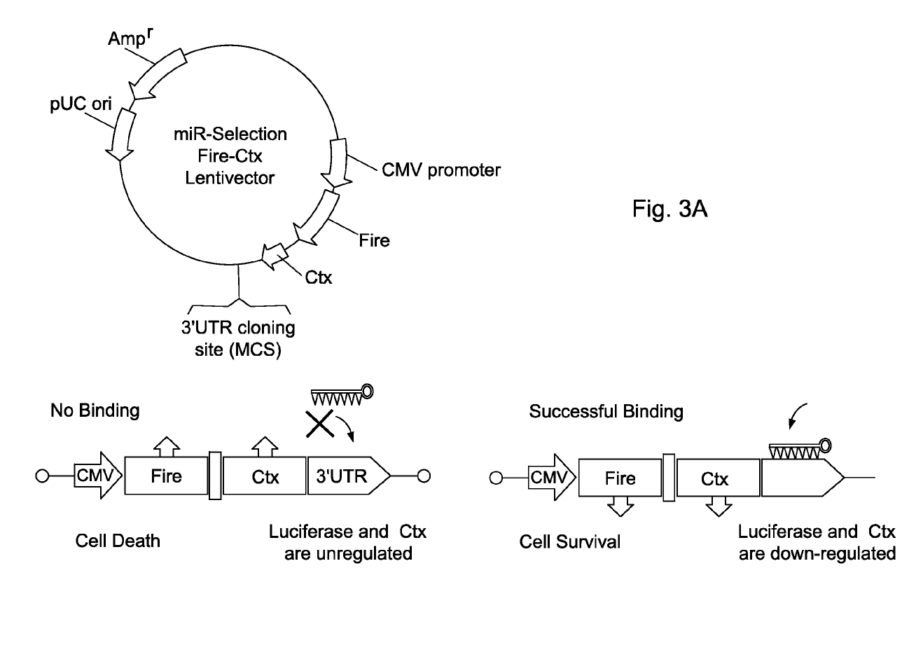

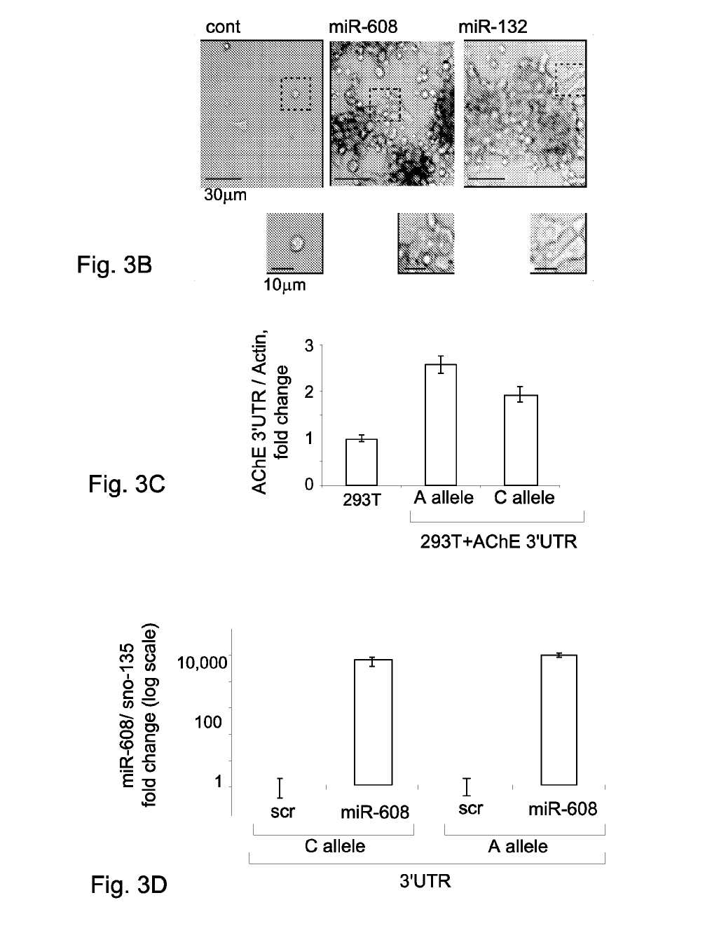

FIG. 3A shows a schematic presentation of the above vector, used for analyzing miR binding to 3′-UTR region of AChE (termed “miR-Selection Fire-Ctx Lentivector”). As illustrated in the lower panel of FIG. 3A, successful binding of miR to the 3′-UTR region of AChE, situated downstream to the upstream to the cytotoxic sensor and firefly luciferase is expected to result in down-regulation of these genes and in cell survival. Micrographs showing cell survival are shown in FIG. 3B.

As presented in FIG. 2D, cells expressing either miR-608 or miR-132 survived and showed 55% and 45% reduction in luciferase activity, respectively, (n=6, one-way ANOVA: p=0.01, p=0.008), reflecting functional miR/target interactions. In contrast, as shown in FIG. 3B, left panel, cells infected with a negative control lentivirus died.

In addition, as demonstrated in FIG. 2E, human-originated U937 cells infected with miR-608 or miR-132 lentiviruses secreted less AChE compared to control cells (by 21.4% and 15.1%, respectively, n=5, one-way ANOVA: p=0.007, p=0.003). Together these results validate miR-608 as a potent AChE suppressor.

Stably transfected 293T cells that do not express AChE [27] carrying comparable copy numbers of either the major or the minor allele (SNP rs17228616) were also prepared, as shown in FIG. 3C. As shown in FIG. 2F, when the cells were co-transfected with miR-608 or control plasmid, only cells carrying the prevalent AChE 3′UTR allele (i.e. the “major allele”) showed reduced luciferase activity (by 44%, n=5, one-way ANOVA:p<0.001), indicating that the minor allele of rs17228616 (i.e. C2098A) reduces miR-608/AChE interaction. As demonstrated in FIG. 3D, the copy number of the transfected miR-608 was comparable in both cell types.

### Example 3

### The Minor Rs17228616 Allele (C2098A) Disrupts AChE/miR-608 Interaction

Bioinformatics analysis (RNAhybrid, http://bibiserv.techfak.uni-bielefeld.de/rnahybrid/) predicted relatively tight binding for both the C-allele (also denoted herein as the “major allele”) and A-allele (also denoted herein as the “minor allele”) sequences (−31.4 and −25.8 Kcal/mol), comparable to the interactions of CDC42 (−26.4 Kcal/mol) and miR-132/AChE (−17.3 Kcal/mol) (see FIG. 4A and FIG. 4B). In order to achieve a quantifiable approach for studying the interactions of miR-608 with targets thereof, an in vitro surface plasmon resonance (SPR) assay was adapted for hybridization tests [28].

Since miR-target interactions may involve longer regions than the seed itself [3], biotin-linked 30-mer RNA sequences of the corresponding regions in the major C-allele of AChE or CDC42 were immobilized onto SPR chips and injected with a 25-mer RNA miR-608 oligonucleotide. In parallel, biotin-linked 30-mer RNA sequence of the corresponding region in the minor A-allele of AChE was immobilized onto another SPR chip. The outcome interaction was compared to that of miR-132 with its AChE target area. As shown in FIG. 4C and FIG. 4D, approximately 15-fold reduction in the affinity between miR-608 and the minor A-allele compared to affinity between miR-608 and the major C-allele AChE sequences was demonstrated (K_{D} of 3.1 vs 50.9 nM, in FIG. 4C and FIG. 4D and comparable K_{D}s in FIG. 4F). These results clearly demonstrate that the A-allele interferes with miR-608/AChE interaction but does not completely ablate it.

The interaction between CDC42 and miR-608 presented an intermediate affinity (15.8 nM, FIG. 4E), close to that of miR-132/AChE binding (18.8 nM, FIG. 4F), predicting a hierarchical binding preference of miR-608 to the C-allele AChE, CDC42 and the A-allele AChE target site (FIG. 4G and FIG. 4H). Similar results are shown in FIG. 4J.

A comparison of the interaction between miR-132 and AChE-R and between miR-132 and AChE-S presented higher affinity between miR-132 and AChE-R (8.2 nM, FIG. 4K, right panel), compared to miR-132/AChE-S binding (18.78 nM, FIG. 4K, left panel).

### Example 4

### The Minor rs17228616 Allele (C2098A) Elevates AChE while Reducing CDC42 and IL6 in Vitro and In Vivo

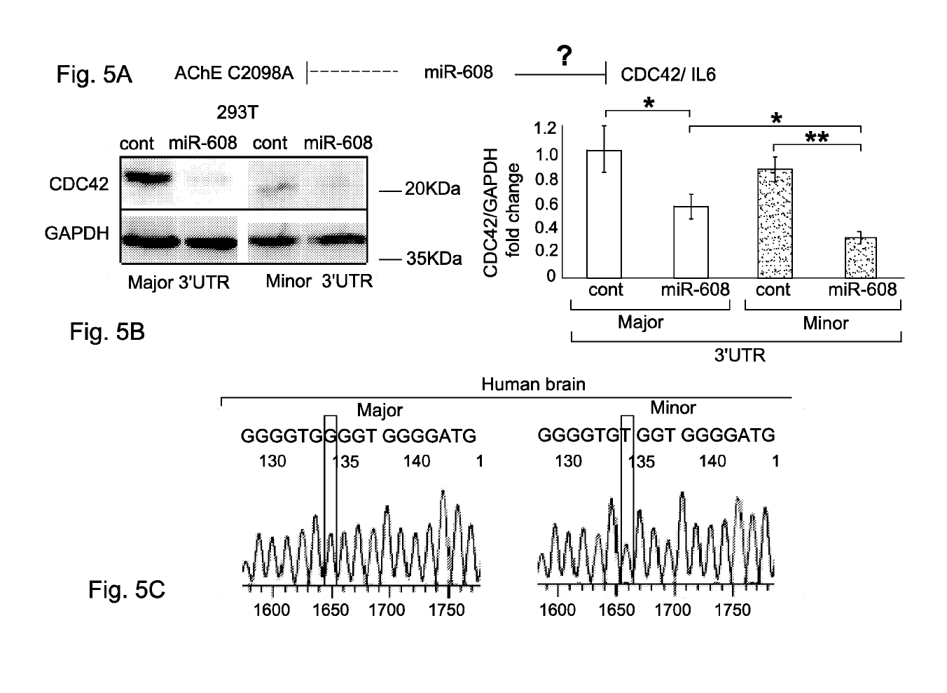

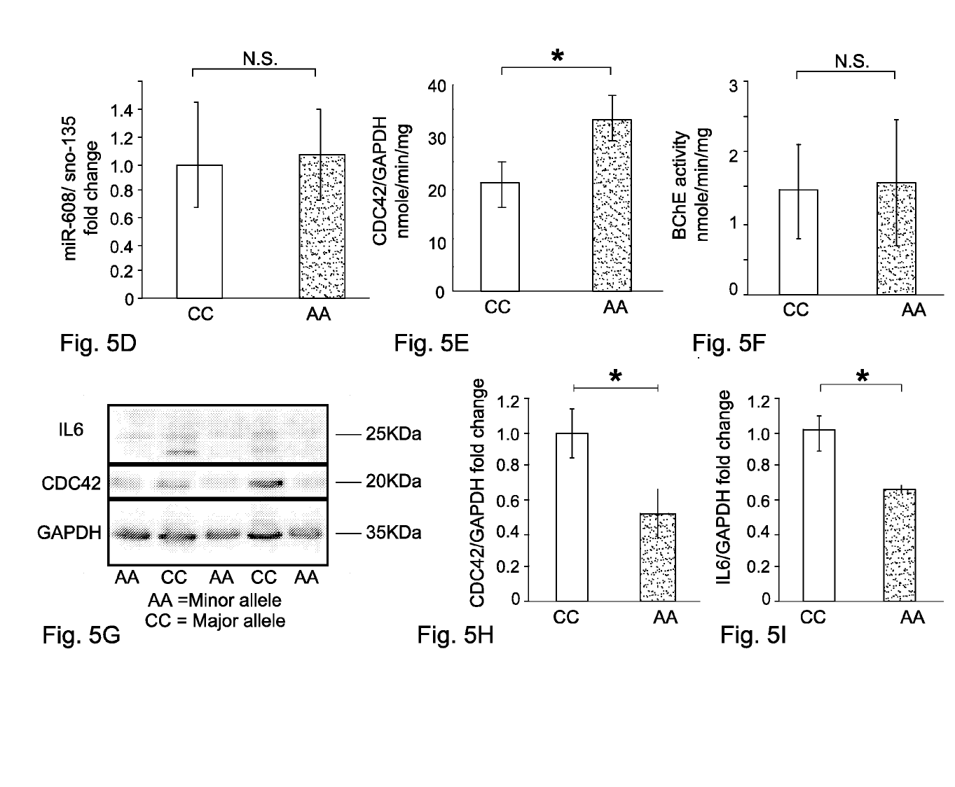

Predictably, miR-608 shows thousands of potential targets (miRwalk: http://www.umm.uni-heidelberg.de/apps/zmf/mirwalk). Of those, the validated miR-608 targets Rho GTPase CDC42 [29] and interleukin-6 (IL6) [30] emerged as those likely to reflect changes in anxiety and parasympathetic signaling. Specifically, the impaired interaction of the AChE A-allele (namely, C2098A) with miR-608 predicted that the presence of this allele would both weaken AChE suppression and free more miR-608 molecules for suppressing other targets, such as CDC42 and IL6, as schematically illustrated in FIG. 5A. Correspondingly, miR-608 transfected HEK293T cells carrying the A-allele 3′UTR AChE sequence (i.e. C2098A) showed intensified CDC42 suppression compared to those carrying the major C-allele (n=6, one-way ANOVA: P<0.05, FIG. 5B).

Both CDC42 and IL6 are expressed in the human brain, where they are involved in GABAergic neurotransmission and inflammatory reactions, respectively [31, 32]. In order to compare the brain levels of CDC42 and IL6, adult brain samples from apparently healthy homozygotes for the minor and major rs17228616 alleles were used. Nucleotide sequence analysis of brain samples is schematically presented in FIG. 5C (where the indicated regions are complementary to those of the major and minor rs17228616 alleles). The analysis of the brain samples is summarized in Table 5 below (samples obtained from The Netherlands Brain Bank [33]).

TABLE 3
post mortem human samples from the Netherland Brain Bank

| NBB | Autopsy | | | BRAAK | | | | | AChE |
|---|---|---|---|---|---|---|---|---|---|
| number | number | sex | age | number | PMD | pH | apoE | Diagnosis | C2098A |
| 2001-016 | S01/054 | M | 77 | 1 | 08:25 | 7.19 | 32 | Non- | No |
| | | | | | | | | demented | |
| | | | | | | | | control | |
| 2009-005 | S09/009 | M | 82 | 1 | 05:10 | 6.75 | | Non- | Yes |
| | | | | | | | | demented | |
| | | | | | | | | control | |
| 2005-055 | S05/195 | M | 84 | 1 | 07:05 | 5.90 | 33 | Non- | No |
| | | | | | | | | demented | |
| | | | | | | | | control | |
| 2003-035 | S03/084 | M | 96 | 1 | 06:30 | 6.65 | 33 | Non- | No |
| | | | | | | | | demented | |
| | | | | | | | | control | |
| 97-115 | S97/266 | F | 89 | 4 | 6:55 | 6.52 | 33 | Non- | Yes |
| | | | | | | | | demented | |
| | | | | | | | | control | |
| 01-008 | S02/016 | M | 62 | 0 | 9:35 | 6.58 | 33 | Non- | Yes |
| | | | | | | | | demented | |
| | | | | | | | | control | |

The A-allele is relatively abundant, particularly in African-originated populations (Frequency of the A allele in individuals with African ancestry (YRI, HapMap population)=0.323; Frequency in Europeans (CEU)=0.04). This enabled the identification of brain samples from three matched pairs of homozygotes for these two alleles. As demonstrated in FIG. 5D, although homozygotes to the major C and the minor A alleles (CC and AA, respectively) showed indistinguishable miR-608 levels, samples homozygous for the minor allele (AA) presented 65% more hydrolytic activity of AChE (Student's t test: p<0.05), but not of the homologous enzyme butyrylcholinesterase (BChE) (see FIG. 5E and FIG. 5F, respectively) [34, 35], compared to homozygote major allele tissues (CC), demonstrating specificity of the induced change.

In addition, immunoblot analyses presented in FIG. 5G and the respective quantification thereof presented in FIGS. 5H and 5I further showed conspicuously lower levels of IL6 and CDC42 in the tissues homozygous for the minor allele (Student's t test: p<0.05 for both), indicating that A-allele-related reduction in AChE suppression can result in increased suppression of other targets of the relevant miR.

### Example 5

### Heterozygotes and Homozygotes for the Minor Rs17228616 A-Allele Show Reduced Cortisol and Elevated Blood Pressure

Anxiety [8, 31, 36] and parasympathetic phenotypes were studied in the HERITAGE Family Study cohort (HEalth, RIsk factors, exercise Training And GEnetics) of young, healthy adults, of Caucasian or African-American ethnic origins [8]. Table 4 and Tables 5A-B below summarize population characteristics.

TABLE 4
HERITAGE Family Study sample characteristics

| | Non carriers | Carriers | p-value |
|---|---|---|---|
| N (SD) | 292 | 76 | |
| Age, years | 34.8 (13.5) | 34.6 (12.6) | 0.913 |
| Gender, % male | 44.2 | 31.6 | 0.047 |
| BMI, kg/m² | 26.3 (5.5) | 27.7 (5.8) | 0.060 |
| Systolic blood | 117.8 (11.3) | 123.4 (12.8) | <0.001 |
| pressure, mmhg | | | |
| diastolic blood | 67.5 (8.4) | 73.5 (8.3) | <0.001 |
| pressure, mmhg | | | |
| Heart rate, bytes/min | 65.6 (8.7) | 67.5 (9.4) | 0.095 |
| State anxiety score | 34.9 (12.0) | 37.1 (12.5) | 0.164 |
| Trait anxiety score | 33.8 (8.5) | 37.0 (9.5) | 0.005 |
| Cortisol, nmol/L | 429.1 (206.3) | 328.5 (123.1) | <0.001 |
| C-reactive protein, | 0.254 (0.373) | 0.452 (1.09) | 0.025 |
| mg/L | | | |
| TNF | 2.07 (0.6) | 2.6 (2.9) | 0.216 |
| AChE activity, nmol | 348.8 (158.9) | 145.7 (17.5) | 0.390 |
| hyd/min/ml | | | |

TABLE 5A
population characteristics by ethnic origin (African American)

| | Non carriers | Carriers | p-value |
|---|---|---|---|
| N (SD) | 96 | 63 | |
| Age, years | 32.0 (10.2) | 34.0 (11.9) | 0.277 |
| Gender, % male | 71.9 | 71.4 | 0.951 |
| BMI, kg/m² | 28.5 (6.46) | 28.2 (6.08) | 0.745 |
| Systolic blood | 121.1 (11.8) | 123.7 (13.3) | 0.208 |
| pressure, mmhg | | | |
| diastolic blood | 71.8 (8.0) | 73.8 (8.5) | 0.151 |
| pressure, mmhg | | | |
| Heart rate, bytes/min | 67.4 (8.4) | 67.9 (9.3) | 0.552 |
| State anxiety score | 35.6 (10.5) | 36.8 (12.6) | 0.489 |
| Trait anxiety score | 35.5 (9.8) | 37.0 (9.2) | 0.365 |
| Cortisol, nmol/L | 398.8 (202.4) | 328.4 (134.2) | 0.022 |
| C-reactive protein, | 0.322 (0.454) | 0.514 (1.21) | 0.284 |
| mg/L | | | |
| TNF | 1.98 (0.53) | 2.62 (3.21) | 0.297 |
| AChE activity, nmol | 305.0 (143.8) | 324.6 (141.4) | 0.422 |
| hyd/min/ml | | | |

TABLE 5B
population characteristics by ethnic origin (Caucasian)

| | Non carriers | Carriers | p-value |
|---|---|---|---|
| N (SD) | 196 | 13 | |
| Age, years | 36.2 (14.6) | 37.9 (15.5) | 0.686 |
| Gender, % male | 48.0 | 53.8 | 0.681 |
| BMI, kg/m² | 25.2 (4.7) | 25.2 (3.8) | 0.994 |
| Systolic blood | 116.3 (10.7) | 121.4 (11.0) | 0.099 |
| pressure, mmhg | | | |
| diastolic blood | 65.6 (7.9) | 71.3 (6.5) | 0.011 |
| pressure, mmhg | | | |
| Heart rate, bytes/min | 64.8 (8.7) | 65.8 (10.8) | 0.702 |
| State anxiety score | 34.5 (12.8) | 38.1 (12.0) | 0.332 |
| Trait anxiety score | 32.9 (7.6) | 37.0 (11.0) | 0.211 |
| Cortisol, nmol/L | 442.9 (207.1) | 333.9 (63.0) | <0.001 |
| C-reactive protein, | 0.225 (0.331) | 0.202 (0.161) | 0.807 |
| mg/L | | | |
| TNF | 2.1 (0.63) | 2.4 (0.42) | 0.369 |
| AChE activity, nmol | 370.3 (162.4) | 346.6 (169.5) | 0.626 |
| hyd/min/ml | | | |

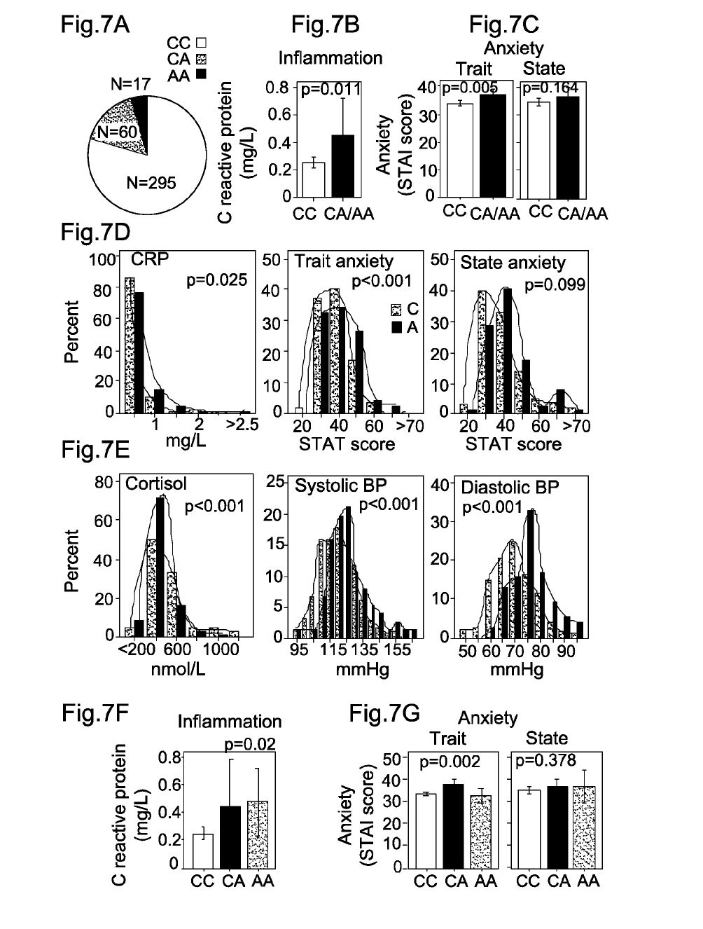

As schematically summarized in FIG. 6A, genotyping indicated that this cohort includes 63 and 13 homozygotes or heterozygotes for the minor A-allele and 96 and 196 homozygotes for the major C-allele in the African-American and Caucasian groups, respectively. Association analysis was done separately for the African-American and Caucasian samples and was combined together using meta-analysis. In support of the working hypothesis of the present inventors, and without being bound by any theory, carriers of the minor A-allele showed reduced serum cortisol levels (FIG. 6B) and higher systolic (FIG. 6C) and diastolic (FIG. 6D) blood pressure compared to homozygotes of the major C-allele (p=9.77×10⁻⁸; p=0.05; p=0.0031), in spite of their young age and generally good health [Sklan, E. H. et al. PNAS 101:5512-5517 (2004)]. These results are also presented in FIG. 7.

AChE is up regulated in anxiety [36] and its elevated levels could suppress ACh levels thus intercepting ACh blockade of inflammation [37]. In comparison, the miR-608 target CDC42 interacts with collybistin in GABAergic neurons and is actively involved in formation of the anxiolytic GABA_{A} receptor synapse [31, 38]. These two pathways may additively increase anxiety and parasympathetic signaling which affects blood pressure [39], as seen in homozygotes and heterozygotes for the minor A-allele who display both AChE increases and CDC42 decreases. Reduced IL6 in the brain may likewise be involved in anxiety [37], as schematically presented in FIG. 6E. Other as-yet non-validated targets of miR-608, and downstream changes in more miRs and their targets, could also contribute to the complex phenotype of elevated anxiety and parasympathetic function.

Of note, the higher incidence of the A-allele in African-Americans compared to Caucasians coincides with a genome-wide association study (GWAS) that reported a significant association between a SNP in complete correlation with rs17228616 and hypertension in African-Americans [40]. The question if these SNPs also involve elevated risk of other aging-related diseases awaits further studies.

### Example 6

### Human Individuals Heterozygotes and Homozygotes for the Minor Allele Showed Reduced Cognitive Recovery Post Stroke

As demonstrated in FIG. 8A, subjects heterozygotes and homozygotes for the minor allele of SNP rs17228616, showed similar cognitive performance upon stroke hospitalization to patients with major allele (Global cognitive score, p=0.148, Memory p=0.371, Executive function p=0.354).

Surprisingly, two years following acute ischemic stroke, the cognitive performance of these subjects were re-tested and as demonstrated in FIG. 8B, while subjects homozygotes for the major allele gained cognitive performance, subjects heterozygotes and homozygotes for the minor allele showed reduced cognitive recovery (i.e. cognitive deterioration). It is hence anticipated that inhibiting AChE activity in such patients may improve their recovery post ischemic stroke.

These results clearly establish the feasibility of using the methods of the invention for early prognosis of high-risk for mal-recovery. Moreover, such methods enable determination of the appropriate treatment regimen by selecting the appropriate patients population that may benefit from a specific therapeutic regimen.

### Example 7

### miR-125b Silencing SNP in the AChE 3′-UTR Shows Differential Effects on Inflammation and on Anxiety

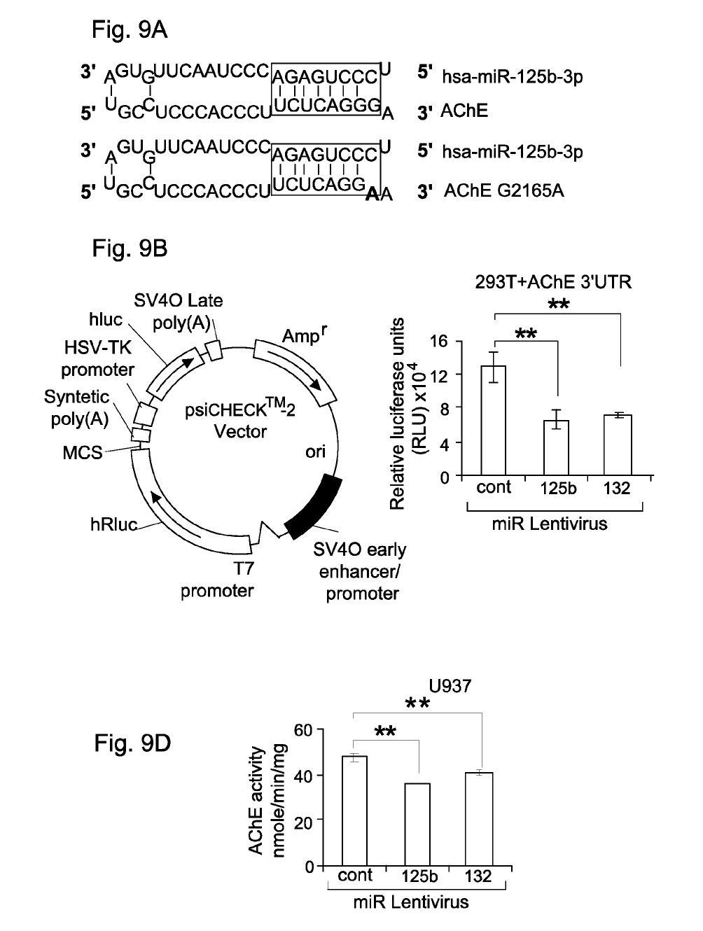

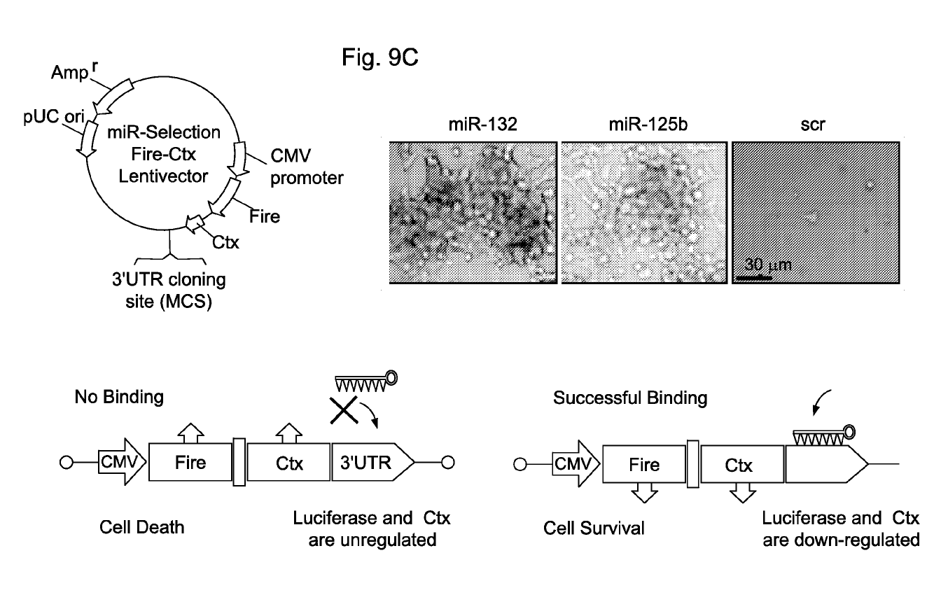

The inventors have further analyzed the potential of SNP rs17228602 in modulating binding of miR-125b, to the 3′-UTR of AChE. As demonstrated in FIG. 9, miR-125b is as active as miR-132 and miR-608 in suppressing luciferase activity associated with the AChE 3′-UTR. More specifically, the direct interaction of miR-125b with the AChE transcript was tested using lentiviruses encoding miR-125b. As shown in FIG. 9, miR-132, -125b and -608 were similarly effective in suppressing AChE activity in infected human macrophage U937 cells, validating AChE as a functional target of all three miRs.

The capacities of two different miR-interrupting SNPs in the AChE 3′-UTR to affect anxiety and inflammation were next measured for the corresponding parameters in carriers of SNPs 11 (G2165A of SNP rs17228602) and 15 (C2098A of SNP rs17228616) which can potentially interrupt the functioning of miR-125b and -608, respectively.

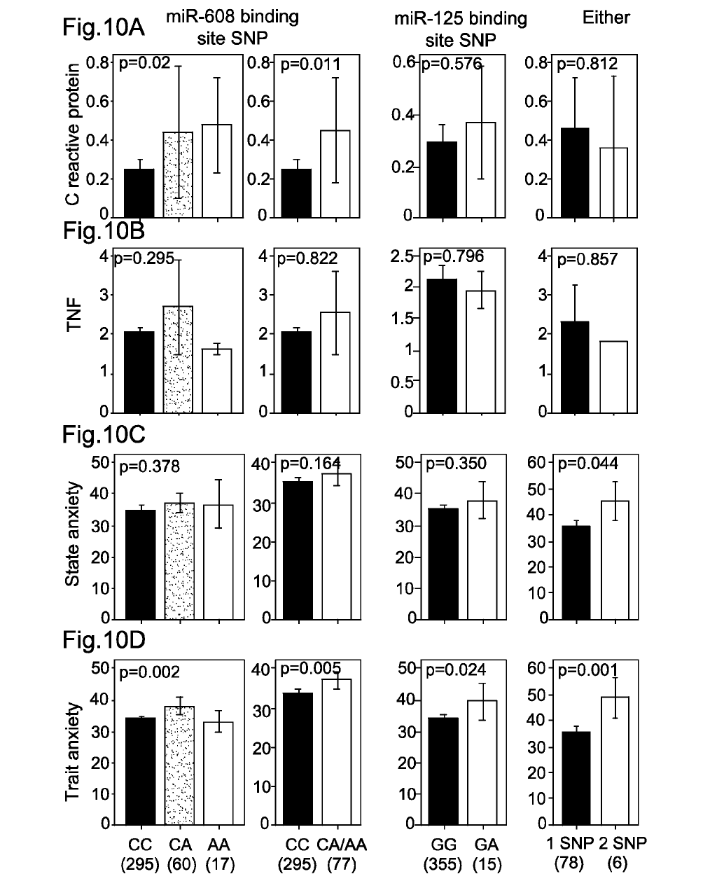

FIG. 10 presents the average outcome for the inflammatory biomarkers C reactive protein (CRP) and tumor necrosis factor (TNF) in the serum, and of state and trait anxiety, for homozygote and heterozygote carriers (or both) of SNP 15 (C2851A or C2098A of SNP rs17228616), and of the heterozygote carriers of SNP 11 (G2165A of SNP rs17228602). For the relatively abundant SNP 15 (76 out of the 372 tested), one or two copies of the mutation sufficed to significantly elevate CRP but not TNF levels; in comparison, SNP 11 caused no significant difference either in CRP or in TNF. This could reflect less potent effect of this SNP on the interaction of miR-125b with AChE, or on the effect of changes in AChE and/or in other targets of these SNPs on inflammation, or both. Likewise, both SNP 11 and SNP 15 sufficed to up-regulate trait, but not state anxiety, and when combined together, these two SNPs caused yet higher elevation (FIG. 10D). This could possibly imply that miR-125b's effect on anxiety is more potent than its effect on inflammation.

It should be noted that the complexity of miR-target interactions merits special attention since inherited, acquired, or therapeutic interference with such interactions may contribute to human diversities and modify phenotypes due to cumulative effects of multiple targets.

### Example 8

### Expose Cultured Stressed Cells to Blocking or Stimulating Agents and Test for Corresponding Inflammation Readouts

miR profiles are cell type-specific; therefore, different miRs will react to similar insults of distinct cell types, and different targets may be affected. The miR changes in insulted cells of neuronal and immune origins are characterized and the various suppressor tools for efficacy in preventing inflammatory outcomes is tested. Primary neurons and splenic macrophages are used, following the induction of threat signals to the brain (by mild foot shock) or to the periphery (by bacterial endotoxin) of both wild type (as control) and conditional transgenic mice with tetracycline-induced excess of miR-132 in peripheral tissues. Alternatively, intra-peritoneal injection of wild type mice with an anti-miR-132 oligonucleotide is performed, which was found to reduce miR-132 and elevate AChE levels in peripheral tissues [41]. The inflammation measurements to be employed are as described above.

1-41. (canceled)

42. A method for the diagnosis of a genetic predisposition of a human subject for at least one AChE-signaling associated disorder, said method comprises the steps of:
a. genotyping in a biological sample of said subject at least one single nucleotide polymorphism (SNP) in the 3′UTR of the acetyl choline esterase (AChE) gene, wherein said SNP is at least one of rs17228616, rs17228602, rs17883268, rs17235010, rs17228609, rs147386700, rs11554090, rs139429533, rs145048252, rs151107784, rs7636, rs149813374, rs145811992, rs113533105, rs148991713, rs3028261, rs145683970, rs61729577, rs74418820, rs41281001, rs138349745, rs116131706, rs144995873, rs17880119, rs17228588, rs8286, rs141785210, rs150598944, rs17881163, rs142818130, rs147370685, rs17228581, rs144986926, rs143875983, rs113406515, rs142452543, rs151335006, s139672629, rs144426398, rs77109413, rs146564868, rs141299237, rs17885778, rs145703834, rs61729575, rs1056867, rs114367422, rs143172740, rs17228574, rs17234982, rs114782198, rs116298479, rs13246682, rs17881553, rs17886728, rs1799806, rs11554090, rs139429533, rs145048252, rs151107784, rs7636, rs149813374, rs14581192, rs113533105, rs148991713, rs3028261, rs145683970, rs61729577, rs74418820, rs41281001, rs138349745, rs116131706, rs144995873, rs17880119, rs17228588, rs8286, rs141785210, rs150598944, rs17881163, rs142818130, rs147370685, rs17228581, rs144986296, rs143875983, rs113406515, rs142452543, rs151335006, rs139672629, rs144426398, rs77109413, rs146564868, rs141299237, rs17885778, rs145703834, rs61729575, rs1056867, rs114367422, rs143172740, rs17228574, rs17234982, rs114782198, rs116298479, rs13246682 and rs17881553;
b. identifying in the genotyped sample of step (a); identifying in the genotyped sample of step (a) at least one SNP that modulates the binding affinity of at least one of miR-608, miR-125b-3p, miR-4319, miR-125a-5p, miR-761, miR-214, miR-298 and miR-4283 to the 3′UTR of the AChE gene;
wherein identifying the presence of at least one of A at rs17228616, A at rs17228602, C at rs17883268 and A at rs17235010 in at least one allele of the AChE gene indicates that said subject belongs to a predetermined population associated with a genetic predisposition for at least one AChE-signaling associated disorder, thereby diagnosing a genetic predisposition of said subject for said disorder.

43. The method according to claim 42, wherein the presence of A allele at rs17228616 in said subject leads to at least one of an increase in the level and catalytic activity of AChE and decrease in the expression level of at least one other gene target of miR-608, said target of miR-608 is at least one of the Rho GTPase CDC42 and Interleukine-6 (IL-6), and wherein identifying the presence of at least one A allele at rs17228616 indicates that said subject belongs to a predetermined population associated with a genetic predisposition for at least one AChE-signaling associated disorder.

44. The method according to claim 42, for the diagnosis and prognosis of mal-recovery of a mammalian subject from at least one AChE-signaling associated disorder, wherein said method comprises the steps of: genotyping in a biological sample of said subject SNP rs17228616; wherein identifying the presence of at least one A allele at rs17228616 indicates that said subject belongs to a predetermined population associated with a mal-recovery from said AChE-signaling associated disorder.

45. The method according to claim 42, wherein the presence of at least one A allele at rs17228602 in said subject leads to at least one of an increase in the level and catalytic activity of AChE and decrease in the expression level of at least one other gene target of miR-125b-3p, and wherein identifying the presence of at least one A allele at rs17228602 in said genotyped sample of (a) indicates that said subject belongs to a predetermined population associated with a genetic predisposition for at least one AChE-signaling associated disorder.

46. The method according to claim 42, wherein said AChE-signaling associated disorders is further characterized with at least one of elevated blood pressure (diastolic and systolic), elevated anxiety (trait and state), reduced cortisol levels and elevated inflammation, and wherein said disorders is any one of anxiety, hypertension, immune-related disorders and any related conditions.

47. The method according to claim 42, for the diagnosis and prognosis of an AChE-signaling associated disorder in a human subject, said method comprises the step of:
a. genotyping in a biological sample of said subject at least one SNP in the 3′UTR of the AChE gene, wherein said SNP is at least one of rs17228616, rs17228602, rs17883268, rs17235010 and any SNP of Tables 1 and 2;
b. identifying in said genotyped sample of (a) at least one SNP that modulates the binding affinity of at least one of miR-608, miR-125b-3p, miR-4319, miR-125a-5p, miR-761, miR-214, miR-298 and miR-4283 to the 3′UTR of the AChE gene;
wherein identifying the presence of at least one of A at rs17228616, A at rs17228602, C at rs17883268 and A at rs17235010 in at least one allele of the AChE gene indicates that said subject belongs to a predetermined population associated with said at least one AChE-signaling associated disorder, thereby diagnosing said disorder in said subject.

48. The method according to claim 47, wherein said method further comprises the steps of:
c. determining in at least one biological sample of a subject displaying the presence of at least one of A at rs17228616, A at rs17228602, C at rs17883268 and A at rs17235010 in at least one allele of the AChE gene, as identified in step (b), at least one of:
(i) at least one of the level of expression and the catalytic activity of AChE in said sample, to obtain at least one of an expression value or an activity value; and
(ii) the expression level of at least one gene target of at least one of miR-608, miR-125b-3p, miR-4319, miR-125a-5p, miR-761, miR-214, miR-298 and miR-4283 in said sample to obtain an expression value of said at least one gene target;
d. determining at least one of:
(i) if the expression or the activity value of AChE obtained in step (ci) is any one of, positive or negative with respect to a predetermined standard expression value or activity value of said AChE or to an expression value or activity value of AChE in at least one control sample; and
(ii) if the expression value of at least one gene target of at least one of miR-608, miR-125b-3p, miR-4319, miR-125a-5p, miR-761, miR-214, miR-298 and miR-4283 obtained in step (cii) is any one of, positive or negative with respect to a predetermined standard expression value of said at least one gene target of at least one of miR-608, miR-125b-3p, miR-4319, miR-125a-5p, miR-761, miR-214, miR-298 and miR-4283, or to an expression value of said target genes in at least one control sample;
wherein at least one of, a positive expression or activity value of said AChE and a negative expression value of at least one gene target of at least one of miR-608, miR-125b-3p, miR-4319, miR-125a-5p, miR-761, miR-214, miR-298 and miR-4283, indicates that said subject belongs to a predetermined population associated with said at least one AChE-signaling associated disorder.

49. The method according to claim 48, comprising:
a. genotyping in a biological sample of said subject SNP rs17228616;
b. identifying the presence or the absence of C2098A of SNP rs17228616 in at least one allele of the AChE gene in said genotyped sample of (a);
c. determining in at least one biological sample of a subject displaying the presence of A at rs17228616 in at least one allele of the AChE gene, as identified in step (b), at least one of:
(i) at least one of the level of expression and the catalytic activity of AChE in said sample, to obtain at least one of an expression value or an activity value of said AChE; and
(ii) the expression level of at least one gene target of miR-608 in said sample to obtain an expression value of said at least one gene target;
d. determining at least one of:
(i) if the expression or the activity value of AChE obtained in step (ci) is any one of, positive or negative with respect to a predetermined standard expression value or activity value, or to an expression value or activity value of AChE in at least one control sample; and
(ii) if the expression value of at least one gene target of miR-608 obtained in step (cii) is any one of, positive or negative with respect to a predetermined standard expression value of said at least one gene target of miR-608, or to an expression value of said target genes in at least one control sample;
wherein the presence of at least one A allele at rs17228616 in said genotyped sample of step (a), and wherein at least one of, a positive expression or activity value of said AChE and a negative expression value of at least one gene target of miR-608, indicate that said subject belongs to a predetermined population associated with said at least one AChE-signaling associated disorder.

50. The method according to claim 49, wherein said target of miR-608 is at least one of CDC42 and IL-6, and wherein said method further comprising at least one of:
(iii) determining in at least one biological sample of a subject displaying the presence of A at rs17228616 in at least one allele of the AChE gene, as identified in step (b), at least one of blood pressure (diastolic and systolic), anxiety (state and trait), serum levels of TNF-α, serum levels of CRP and serum cortisol levels; and
(iv) determining the ethnic group of said subject, wherein said ethnic group is any one of (i) African; and (ii) Caucasian.

51. The method according to claim 47, comprising:
a. genotyping in a biological sample of said subject SNP rs17228602;
b. identifying the presence or the absence of G2165A of SNP rs17228602 in at least one allele of the AChE gene in the genotyped sample of step (a);
c. determining in at least one biological sample of a subject displaying the presence of A at rs17228602 in at least one allele of the AChE gene, as identified in step (b), at least one of:
(i) at least one of the level of expression and the catalytic activity of AChE in said sample, to obtain at least one of an expression value or an activity value; and
(ii) the expression level of at least one gene target of miR-125b-3p in said sample to obtain an expression value of said at least one gene target;
d. determining at least one of:
(i) if the expression or the activity value of AChE obtained in step (ci) is any one of, positive or negative with respect to a predetermined standard expression value or activity value of AChE or to an expression value or activity value of AChE in at least one control sample; and
(ii) if the expression value of at least one gene target of miR-125b-3p obtained in step (cii) is any one of, positive or negative with respect to a predetermined standard expression value of said at least one gene target of 125b-3p or to an expression value of said target genes in at least one control sample;
wherein the presence of at least one A allele at rs17228602, and wherein at least one of, a positive expression or activity value of said AChE and a negative expression value of at least one gene target of 125b-3p, indicates that said subject belongs to a predetermined population associated with an AChE-signaling associated disorder.

52. The method according to claim 47, wherein said AChE-signaling associated disorders is further characterized with at least one of elevated blood pressure (diastolic and systolic), elevated anxiety (trait and state), reduced cortisol levels and elevated inflammation, said disorders is any one of anxiety, immune-related disorders and hypertension and any related conditions.

53. A kit comprising:
a. reagents for genotyping in a biological sample of a mammalian subject at least one SNP in the 3′UTR of the AChE gene, wherein said SNP is at least one of rs17228616, rs17228602, rs17883268 and rs17235010;
b. reagents for determining at least one of:
(i) at least one of the expression level or the catalytic activity of AChE in said sample; and
(ii) the expression level of at least one gene target of at least one of miR-608, miR-125b-3p, miR-4319, miR-125a-5p, miR-761, miR-214, miR-298 and miR-4283; optionally said kit further comprising (iii) regents for determining at least one of blood pressure (diastolic and systolic), anxiety (state and trait), serum levels of TNF-α, serum levels of CRP and serum cortisol levels.

54. The kit according to claim 53, for use in a method for the diagnosis of a genetic predisposition of a mammalian subject for at least one AChE-signaling associated disorder or in a method for the diagnosis and prognosis of at least one AChE-signaling associated disorder in a mammalian subject.

55. The kit according to claim 53, comprising:
a. reagents for genotyping in a biological sample of a mammalian subject SNP rs17228616;
b. reagents for determining at least one of:
(i) at least one of the expression level or the catalytic activity of AChE in said sample; and
(ii) the expression level of at least one gene target of miR-608, said target of miR-608 is at least one of CDC42 and IL-6.

56. A method for the treatment of an AChE-signaling associated disorder in a mammalian subject, said method comprises the step of:
a. genotyping in a biological sample of said subject at least one SNP in the 3′UTR of the AChE gene, wherein said SNP is at least one of rs17228616, rs17228602, rs17883268 and rs17235010;
b. identifying at least one SNP that modulates the binding affinity of at least one of miR-608, miR-125b-3p, miR-4319, miR-125a-5p, miR-761, miR-214, miR-298 and miR-4283 to the 3′UTR of the AChE gene in the genotyped sample of step (a);
c. administering to a subject displaying the presence of at least one of A at rs17228616, A at rs17228602, C at rs17883268 and A at rs17235010 in at least one allele of the AChE gene, as identified in step (b) a therapeutic effective amount of at least one of: (i) at least one antagonist of at least one of miR-608, miR-125b-3p, miR-4319, miR-125a-5p, miR-761, miR-214, miR-298 and miR-4283; and (ii) at least one AChE antagonist.

57. The method according to claim 56, comprising the steps of:
a. genotyping in a biological sample of said subject SNP rs17228616;
b. identifying the presence or the absence of C2098A of SNP rs17228616 in at least one allele of the AChE gene in the genotyped sample of step (a);
c. administering to a subject displaying the presence of at least one A allele at rs17228616, as identified in step (b), a therapeutic effective amount of at least one of: (i) at least one antagonist of miR-608; and (ii) at least one AChE antagonist.

58. The method according to claim 56, wherein said AChE-signaling associated disorders is further characterized with at least one of elevated blood pressure (diastolic and systolic), elevated anxiety (trait and state), reduced cortisol and elevated inflammation, and wherein said disorders is any one of anxiety, an immune-related disorder, hypertension and any related conditions.

59. The method according to claim 42, for assessing the anxiety level of a mammalian subject, said method comprises the steps of:
a. genotyping in a biological sample of said subject SNP rs17228616;
b. identifying the presence or the absence of C2098A of SNP rs17228616 in at least one allele of the AChE gene in the genotyped sample of step (a);
c. determining in at least one biological sample of a subject displaying the presence of A at rs17228616 in at least one allele of the AChE gene, as identified in step (b), at least one of:
(i) at least one of the level of expression and the catalytic activity of AChE in said sample, to obtain at least one of an expression value or an activity value; and
(ii) the expression level of at least one of CDC42 and IL-6 in said sample to obtain an expression value of at least one of CDC42 and IL-6 in said sample;
d. determining at least one of:
(i) if the expression or the activity value of AChE obtained in step (ci) is any one of, positive or negative with respect to a predetermined standard expression value or activity value of AChE, or to an expression value or activity value of AChE in at least one control sample; and
(ii) if the expression value of at least one of CDC42 and IL-6 obtained in step (cii) is any one of, positive or negative with respect to a predetermined standard expression value of said at least one of CDC42 and IL-6 or to the expression value of said at least one of CDC42 and IL-6 in at least one control sample;
wherein at least one of a positive expression or activity value of said AChE and a negative expression value of at least one of CDC42 and IL-6 indicates that said subject belongs to a predetermined population associated with a specific level of anxiety.

60. The method according to claim 42, for monitoring at least one of, the efficacy of a treatment of a mammalian subject suffering from at least one AChE-signaling associated disorder, with a therapeutic agent and the disease progression, said method comprises the steps of:
a. genotyping in a biological sample of said subject at least one of rs17228616, rs17228602, rs17883268 and rs17235010;
b. identifying the presence or the absence of at least one of A at rs17228616, A at rs17228602, C at rs17883268 and A at rs17235010 in at least one allele of the AChE gene in the genotyped sample of step (a);
c. determining in at least one biological sample of a subject displaying the presence of at least one of A at rs17228616, A at rs17228602, C at rs17883268 and A at rs17235010 in at least one allele of the AChE gene, as identified in step (b), at least one of:
(i) at least one of the level of expression and the catalytic activity of AChE in said sample, to obtain at least one of an expression value or an activity value; and
(ii) the expression level of at least one gene target of at least one of miR-608, miR-125b-3p, miR-4319, miR-125a-5p, miR-761, miR-214, miR-298 and miR-4283 in said sample to obtain an expression value of at least one of said gene targets in said sample;
d. repeating step (c) to obtain expression values of at least one of AChE and said at least one gene target, for at least one more temporally-separated test sample; wherein at least one of said temporally separated sample is obtained after the initiation of said treatment;
e. calculating the rate of change of said expression values of at least one of AChE and said at least one gene target between said temporally-separated test samples;
f. determining if the rate of change obtained in step (e) is positive or negative with respect to a predetermined standard rate of change determined between at least two temporally separated samples or to the rate of change calculated for expression values in at least one control sample obtained from at least two temporally separated samples, wherein at least one sample of said at least two samples is obtained after the initiation of said treatment; wherein at least one of a negative rate of change in the expression value of AChE and a positive rate of change at least one of said at least one gene target expression value indicates that said subject exhibits a beneficial response to said treatment; thereby monitoring the efficacy of a treatment with a therapeutic agent and the disease progression.

61. The method according to claim 60, wherein said method further comprises the steps of: determining in at least one biological sample of a subject displaying the presence of at least one of A at rs17228616, A at rs17228602, C at rs17883268, A at rs17228609 and A at rs17235010 in at least one allele of the AChE gene, as identified in step (b), at least one of blood pressure (diastolic and systolic), anxiety (state and trait), serum levels of TNF-α, serum levels of CRP and serum cortisol levels.


SEQUENCE LISTING (63 sequences)

SEQ ID NO: 1 - DNA, 27 bp, Artificial Sequence
  Synthetic
ccatccccaccacaccccgacgtcccc

SEQ ID NO: 2 - DNA, 27 bp, Artificial Sequence
  Synthetic
ggggacgtcggggtgtggtggggatgg

SEQ ID NO: 3 - RNA, 25 nt, Artificial Sequence
  Synthetic
aggggugguguugggacagcuccgu

SEQ ID NO: 4 - RNA, 29 nt, Artificial Sequence
  Synthetic
ccccagacucugcccauccccaccccacc

SEQ ID NO: 5 - RNA, 29 nt, Artificial Sequence
  Synthetic
ccccagacucugcccauccccaccacacc

SEQ ID NO: 6 - RNA, 30 nt, Artificial Sequence
  Synthetic
gccucugcgucuuuuuacucaccaccuuag

SEQ ID NO: 7 - RNA, 22 nt, Artificial Sequence
  Synthetic
uaacagucuacagccauggucg

SEQ ID NO: 8 - RNA, 30 nt, Artificial Sequence
  Synthetic
cugggccgccaauaaacuguuacagccacc

SEQ ID NO: 9 - RNA, 16 nt, Homo sapiens
ccacccuucucaggaa

SEQ ID NO: 10 - RNA, 24 nt, Homo sapiens
cgugccucccacccuucucaggaa

SEQ ID NO: 11 - RNA, 21 nt, Homo sapiens
ugccucccacccuucucagga

SEQ ID NO: 12 - RNA, 18 nt, Homo sapiens
uaacacagacaagcccca

SEQ ID NO: 13 - RNA, 22 nt, Homo sapiens
ccuccucucuccuucccgcugu

SEQ ID NO: 14 - RNA, 23 nt, Homo sapiens
uccuccucucuccuucccgcugu

SEQ ID NO: 15 - RNA, 25 nt, Homo sapiens
uuucuccuccucucuccuucccgcu

SEQ ID NO: 16 - RNA, 24 nt, Homo sapiens
cagacucugcccauccccaccaca

SEQ ID NO: 17 - DNA, 20 bp, Artificial Sequence
  Synthetic
gttcgaccactacagcaagc

SEQ ID NO: 18 - DNA, 19 bp, Artificial Sequence
  Synthetic
cccgctcctgcatagactc

SEQ ID NO: 19 - DNA, 12927 bp, Homo sapiens
tggaacctgtgagaccaggaactgacagccagttggaccagcatgccagggaccatgtcagatggatgtcaggtcataccagggaagatgagtggcaacatggcgttcaatgtactactggtgaattaaattgcatatttcactcctttcagaagcactcaagttcatgggcaaaaaggaatgccaagaaaagtactgatcagttctgatggccgggcgcagtggctcacacctataatcccagcactttgggaggcagaggtgggaggatcgcttgagcccaggagttcaagaccagcctgggcaacatagcaagaccccgtctctacaaattaataaaatatttaaaaattagctgggcatggtggtgccacctgtggtcccagctattcagaaggctgaaggagggttactgcttgagcccaggacttcaaggctgcaatgagatatgatcacaccatttcactccaaccccgggtgacagaatgagaccaaaaaaaaaaaaattctgagcacaagggtatgccacccgcactccactctacccccggccagaaaagaggctaaactaagcaaaaggttatttggcgaattgcatgggaaatactgtatactcgttaaataaataacaaaattgtgaaaataaagtaagactttgtagagacaagctataaaaaatgtgtgaaatgctcataactagcaagagagaaaagggtaaaaaggatggataagccgcaaagagaaatcatcctatcctgtgtaggatgacgtaacttggatccagcaggaaggctggcccaacattaagtaggggacataggaaggggtgggtagagggaggaaagtcctttgtgtggcctcaggctcccaattctgccaggttacaatctcgattaatggggggttgggcaaaggctcatcattaaaggaagagttcggccggatatggttgctcaggcctgtaatcccagcactttgggaggctgaggcaggtggatcacttaaggccaggagtttgagaccagcctggccaacatggtgcaatctcatctctactaaaaatacaaaaaattaaccaggcatggtggtgcgcacctgtaatcccagctactcaggaggctgaggtgggagaatcacttgaacctgggaagtggaggttgcagtgagccgagtgccactgcactccagcctggggcgacagagtgagactctgtcaaataaataaataaataggagatactgttcttcagcccctcctccttcctgctggctggaatgtagatgtgatggctgatgttccagcagccattctatgccatgcggtagaagtcacatgctgaagatggtgcagtaacaagatactgtgcaaggagcaacagggttccgtgtaaagaagcctggaccctgacattgagggcaccatgccaaccctgcaagactcatttctaggtctctttttttttttttttaagacagagtctccctctgtcacccaagctggagtgcagtggcgagatcttggcccactgcaatctccgcctcccaggttcaagcaattcttgtgcctcagcctcccaagtagctaggagtacacgcacgcaccaccataccaagctaatttttgtatttttagtacagacaaggttttaccatgttgcccaggctggtctcaaactcctggcctcaagtgatcctcctgcctcagcctcccaagtagctagaactataagtatgcaccaccacacccagctaattatttttattttttgtagagacagggtctcaccacattggccatgctggcctcatactcctggcctcaagtgatcctctctccttgtcctcccaaagttctgggacgacagtcactgcacctggcccaagaaatacatttctattatttttttttttttttgagacagagtttcgctcttgttgcccaggctggagtacaatggcatgatttccgctcactgcaccctccacctcctgggttcaagcgattctcctgcctcagcctcctgagtagctgggattacaagcatgggccaccatgcccagctaatttttttgtatttttagtagagacagggtttctccatgttggtcaggctggtcttgaactcccaacctcaggtgatctgcctgcctcagcctcccaaaatactgggattacaacaggcatgagccaccatgcccggcctctattatatcttgttaaagccattatttggtttttggtctcatacagctgaattgaatattaaccaataaatagaggaaataagaatcttttgtgttagaagatgtggatcaaggtctgtaatagaagtggggttccctgccaggcatggcagctcatgcctgtaatcccaacactttgggaggctgaggcaggaggatcacctgaggtcaggagttcgagaccagcctggccaacatggtgaaaccccacctctgctaaatatacaaaaattagccaggtgtggtggcaggcgcctctagtcccagctactcaggaggctgggaaaggagaatccttgagcccaggaggcagaggttgcagcgagctgagatggcatcatctctacactctagcctgggcgacagagcgagactctgtctaaaaaaaaaaaatggcgcttccttccaaggtgggagacagagataaaagaggggtagggaaattctctgaattggcagaactggtagtagaagcaaggctcattcctggataaacagaaatggtgagagaagaaatgggaaaaatcaaggacatccctgcagtgacagctgtggagggtggggagtttactatgctagactatgtagggaaacgcctcctggcttgacaggttaggtgcctgtgttgacaacggttgctgagagctcaaggtgaaaagagggatagttccaggaaatgataaccctggtgataaactatccctgccctccctatcttcattgtacaggcctagaaactcgtgacgacagaggttaggtgacacctgcgtggcacagttcgtctggagggtgacaggggcaagccttcatcactagcagggccaaaaatccctaaagcctcaggactggggcaggcctcagagcactcgttcacagtttgtttccatcattttatttttttgcggagaagggagtctcgctatgttgcccaggctagtctccaactcctggactgaagcgatcctcctgtctcggcctcccagagtgctgggattacaggcaccgcactaggggccacacgcggccccatcattttaataattcataattaccacgcttagaaacctgcctcagattcattccccccgcagcaacacccaccatccaccctcctgtcccctcccccatgcacagcccttacatctgctcttcagggatctcaactagggcacatggtctggccagtgaggagccttcaactagtgcggccagaacgctgaaggccggatacaaggagcctcggtgtccaggcaggggatttggggttgcctgagattggggcggggaaagccggagacagtgggcacagatggcgaggggttcacctccccaacacccccaacccagcccagtctccgaggcccgagtggctgctttttgcgccacctggcggccgtctccagcgagcggagcgcgggtgaccgtgcctgcgggacccggttcccgtggggcgagcgggcgtggggcccgcccagcttggagagccgggaaccgcgcggcccagggactggcaagactccggattcaacccctgcgaggccccgcaacccggtgcccgcggtccctggcggcggcgcccgcgtgcccgaggcgctgcggggcgctgcggggcgcgcggcggttagagcgagcgtgtgcgcacgcgcccaccccacgccgcgccccgcaccgccgcgcgcgcgcacgcacgcccgacgctggcctgcgccggccgctctcccacgcggggtctcgggggctcctgagccggccgcctccaggaagcaggcgccgcgcggtattgccgcatgcacctcggtcgtggggaccccgctgcagcagccctgtccacacgggtgaactcctggagggcggggcggggggcaatctgcgcgggtcaggcccctcggagcaggctgggggcgcccgagccaggccgctcccacctgccagccgcgtggccaatgaatgctaggcctggtgatgtcatgccccgaccggaccctggtgacgaaagtccgaagtcacccgtcagggaaccagcacagacccacccgcgggggttccagaagtttccactgccgccgcggagtgcgcctcgcccagcagccttgcgcgtgctaccacgctgtcctgccttctcaggtgtcccaagtcaccccttcctcccccaggggtctcctgtctccatgccctcccacctcatttccactagcgatcccattttacatctccattcccacaggtgtcctttcatccccaggagtgtcccacgtcaccttttctgcacccgtgccacctggacccctaaggggacggtactctgccacactcccacatgcctctcctcccatgccacccgtctgtgtcgtggccggggatgcatgaccccttggccttgcgctaacttcaccagcctcgagcgagaccgacagggtgtccaagtgtgtccgtccgtctgtgaaagtcgtccctgcgtttgtctgtgcctgtcactgcatgaggtcgtccgtctgcgcgtgtctgtctccacgggcttctgtccgtctgtctgtcacgtgggtgtctactccgccgctgcaggtctttgggtcgcgtctgtcactgcgctctgtcgtcggtcggcgtctggcactggagtgcgtctgggccccggggtctccgggtctcggctccaagggagggagagagaggggaggtggccacccgggagagcggggaggggcgggggcggagacagtgggcgggggcgggcgggggcgctgtgaggcccggagggggtgtgtgcggggggccggaggcggcggctgtcagagtcggctcagcctgcgccggggaacatcggccgcctccagctcccggcgcggcccggcccggcccggctcggccgcctcaggtgagtctccctccccgcccgggactctccttaaggcccgggtccggggcaccgtgcgctccccgaggggtccaggcccccctaacgggacggctcccgcttccccgggcctgccccgcgccgccctccgctgccccgcactcgtccggaactcttccccggccgcctcacactcaccgacgcccacgcaggagcccgagcccgcgggaacggccgccgcctctgcgctccgattcccgcggggacccccgagcaccccttccccgatcgctccctagtccggggccactggaagacaccccctaattggggcggtacccgagctgcgcaggttcccggcgccgggctgggcacccccatccccagggccacaagccagcgcccctgcgcagcgccccgcagaggccccctccgaggcccaggacgccccctcccgcggagactccatctctattttttgggaaggggaggctcggcggaagccccgagttataattagcgccactcgggtttcctagttaatctccagcagcaccaccaccccccgctcagggcggcgggggctgggctaggagtgaccggcgggagaggggggagttcccacccggggaattttgatcctttggctggagatgccggaaccgtagcagctgctgcccccaaaatagcgcccccgcccctgcagccggggatctccggagtcccgggaacgcaggcgtcccggctcgcccttcaggcggctctgcaaggccctcgagcccccaggccccctccccaagttcccggctcctggctcggggcggggagcgccgaccttttctcggtctctactgccctccgccggcggcggcgtgcacggcagcttcagacggatggggggctctcgagtcctgggggacgcgggctggcgagcctgtttgctggggctgggctgggagcgtgcgcagctgatgggaacccgcgggcatcagcgctcggtgccagacattggatgagaaggggcagggttgggtggggagtggactaggcaggatttgggggaccccgggacctgaggcttccacaccctgaatgcccagcccaccgccccccagggccccgggtctggccgacccaggagagcggggctgcgcttcaggccgccgcaagctcccccagcggcgctgcccgtccgtgaggccgcgccctgtcgggtggcctgttcgtggactgaggcttctggtgccctcactgcacgagctgggcccggcccagttccgggaagaggtgagggcagtggacccctgcggagcagagagggtgctcctctctgttccctgcatttgtctggatatgggtgtctgcctgatctctttcccctcctgcccgctggcctgtgtccttgtctctgcctgtctccttccctccctccctctctcccctcatctttgccaacctgccccacctcctctgcagctgagcgataacccttgggccgacagtgccctaatctcctccctcctggcttctcgaccgacccttcaccctttccctttctttctcccagcagacgccgcctgccctgcagccatgaggcccccgcagtgtctgctgcacacgccttccctggcttccccactccttctcctcctcctctggctcctgggtggaggagtgggggctgagggccgggaggatgcagagctgctggtgacggtgcgtgggggccggctgcggggcattcgcctgaagacccccgggggccctgtctctgctttcctgggcatcccctttgcggagccacccatgggaccccgtcgctttctgccaccggagcccaagcagccttggtcaggggtggtagacgctacaaccttccagagtgtctgctaccaatatgtggacaccctatacccaggttttgagggcaccgagatgtggaaccccaaccgtgagctgagcgaggactgcctgtacctcaacgtgtggacaccatacccccggcctacatcccccacccctgtcctcgtctggatctatgggggtggcttctacagtggggcctcctccttggacgtgtacgatggccgcttcttggtacaggccgagaggactgtgctggtgtccatgaactaccgggtgggagcctttggcttcctggccctgccggggagccgagaggccccgggcaatgtgggtctcctggatcagaggctggccctgcagtgggtgcaggagaacgtggcagccttcgggggtgacccgacatcagtgacgctgtttggggagagcgcgggagccgcctcggtgggcatgcacctgctgtccccgcccagccggggcctgttccacagggccgtgctgcagagcggtgcccccaatggaccctgggccacggtgggcatgggagaggcccgtcgcagggccacgcagctggcccaccttgtgggctgtcctccaggcggcactggtgggaatgacacagagctggtagcctgccttcggacacgaccagcgcaggtcctggtgaaccacgaatggcacgtgctgcctcaagaaagcgtcttccggttctccttcgtgcctgtggtagatggagacttcctcagtgacaccccagaggccctcatcaacgcgggagacttccacggcctgcaggtaactagtggctagctggcgtgaagctggctcctctgggtcccaacggtccctcccctccctgcagggacccaggcatgagggcttctccaggcccattcccagaagtcccagaagtcctccctgagggctcagatcccagggtggtcagcagggcagacaggaaagccaccatgggtctattttctctttctctgcatccctcccctgatctcgtcctctctctgtccatggttccgggtctgtaactgttcatctctctggctctttgtctgtccatctgtttctgtctacttgtctgtctgtgcctgtcgctccatcccacccccctctccctcacccccaggtgctggtgggtgtggtgaaggatgagggctcgtattttctggtttacggggccccaggcttcagcaaagacaacgagtctctcatcagccgggccgagttcctggccggggtgcgggtcggggttccccaggtaagtgacctggcagccgaggctgtggtcctgcattacacagactggctgcatcccgaggacccggcacgcctgagggaggccctgagcgatgtggtgggcgaccacaatgtcgtgtgccccgtggcccagctggctgggcgactggctgcccagggtgcccgggtctacgcctacgtctttgaacaccgtgcttccacgctctcctggcccctgtggatgggggtgccccacggctacgagatcgagttcatctttgggatccccctggacccctctcgaaactacacggcagaggagaaaatcttcgcccagcgactgatgcgatactgggccaactttgcccgcacagggtcagcagtgcagagggagagaaggctggggaggacaggagtcgggggcgggaggacacacaaaggcagacacacggagacagaaaggacgggtgggacaggggcaggatagaacagccagagagggatatgctcacaaagtagataaaaggggagagaaaaagaaaggagacagagaaggcagagggggtcttgcagagacagaggcaaaggaaagtgaggaggagacgaggtagacatggagggggcggggcacagtggctcacccctgtaattccagccctttgggaggccaaggtgggaggatcccctgaggccaggagtttgagaccagactggacaacataacaagaccccgactcttaaaaagaaaaaaaatacaaaacttagccaggcacggtggctcgtgcctctagtcccagctacttgggaaggtgaggcaggaggattgcttgagcctgggaggttgaggctgcagtgagctatgatggcaccactgcactccagcctgagtggccctatctctggaaaaaaaaaaaaaaaaaaaaaagggagaccaagacaaaggagttggagctggattaagtcaaaccagtgatgattcactagcagtgacacattctctcacacacacaccagtttacctggatcgagaagaggacaatgaaatctagcattggatgggtgttgttaaaaaatgtgatgtttgtagacataggacccgtcctggttatttaggaaagtctcaagacatcctagccatttttttagtgagagaaacaaagatggaacagaaaagctgagggaaacggaaggagacggagcgcttgaggaagggaagaccccaatgaccgcgcacattggagaaagtggtgggagggaggggacgggttctctggggctctccgctgagctgaagagtccgggatcccgtggagttgggggccacctcctcccactgcccgtctggactgagccttcccttccttccgcagggatcccaatgagccccgagaccccaaggccccacaatggcccccgtacacggcgggggctcagcagtacgttagtctggacctgcggccgctggaggtgcggcgggggctgcgcgcccaggcctgcgccttctggaaccgcttcctccccaaattgctcagcgccaccggtatgcaggggccagcgggcagcggctgggaggaggggagtgggagcccgccaggtgtaacccctctcttctccccctagcctcggaggctcccagcacctgcccaggcttcacccatggggaggctgctccgaggcccggcctccccctgcccctcctcctcctccaccagcttctcctcctcttcctctcccacctccggcggctgtgaacacggcctcttcccctacggccacaggggcccctcctctaatgagtggtcggaccgtggggaagggccccactcagggatctcagacctagtgctcccttcctcctcaaaccgagagactcacactggacagggcaggaggagggggccgtgcctcccacccttctcagggacccccacgcctttgttgtttgaatggaaatggaaaagccagtattcttttataaaattatcttttggaacctgagcctgacattggggggaagtgggaggccccggacggggtagcaccccccattggggctataacggtcaaccatttctgtctcttctttttcccccaacctccccctcctgtcccctctgttcccgtcttccggtcattcttttctcctcctctctccttcctgctgtccttctccggccccgcctctgccctcatcctccctctcgtctttcgcacattctcctgatcctcttgccaccgtcccacgtggtcgcctgcatttctccgtgcgtcctccctgcactcatacccccccttcaacccgcccaaatgtccgatccccgaccttcctcgtgccgtcctcccctcccgcctcgctgggcgccctggccgcagacacgctcgacgaggcggagcgccagtggaaggccgagttccaccgctggagctcctacatggtgcactggaagaaccagttcgaccactacagcaagcaggatcgctgctcagacctgtgaccccggcgggacccccatgtcctccgctccgcccggccccctagctgtatatactatttatttcagggctgggctataacacagacgagccccagactctgcccatccccaccccaccccgacgtcccccggggctcccggtcctctgcatgtctcaggctgagctccctcccccgcggtgccttcgcccctctgggctgccaataaactgttacagccacgggagtgtgcgcgactagggagccaggggtagaggcagaacgccggaatcacgggggccgagtctatgcaggagcggggctggagggcaagaaacaggcgagctccgaggcgggcgcaaggcaaaggccaacccctagccctgccctgccgggcggagctcgcgcctgcgtaatgaggcccgcaggcaggctagctggcacggcggaggggaggagaggggaggggaggggaggggaggggaggggaggggcggggcggggcggggcgcggcggagcattgtgggagctcctcggtcggtgccggtcggtggctgcctattgcggcctgcggtgatcaacgaggcccggggagcgcgtccccagtctgcgcgccggtcctgcggcagctggcccaagacccggagccgaaaggaagtgttggagcctgaggtcgctccgcgccgctaggaggacgctgtgcctggcctgggacctccgctcccgcccaccgccctggagccgctgagggacgtccacgtgggcctgtccccgccgagccgcggccctgtccgcctggcgctgctctcgggccactacctctactaccactacggctgcgacggcctggacgaccgcggctggggctgcggctaccgcactctgcagacgctgtgctcgtggccagagggccagcccgcgggcgtacctggactggccgccgtacaggcggccctggaggacatgggcgacaagccccccggcttccggggctcccgggactggatcggctgcgtggaggccagcctctgcctcgctcacttcggagggccccagggacgcctctgccacgtaccccggggagtggggctgcacggggagctggagaggctttactcgcacttcgcagggggtgggggcccagtcatggttgggggggacgcagatgccaggtccaaggccttgctgggagtctgcgtagggtcaggcacggaagcctatgtcctggtattggaccctcactactggggcactccaaaaagccccagtgaactacaggctgctgggtgggtgggctggcaagaggtgagtgcagcctttgaccccaactccttctacaacctgtgcttgaccagccttagctcccaacagcagcagcgcaccttggactgaggacgaagttacagaactgagattctcgggtcccagacacgcacctatgtacctcccactggtgtccctgcaaagcctggcgcttttgacatcaataataaaagtggcagggctgagcaacacctcaggagttactctggaaggatggaggagttatgtaacacacgagagtcaggagccctgtggaagtgcttttattagcagtaaggctgatcgtacaaaaaattctcagagcttcataggacaaggtagtacaagtatggatgatacaggactgaggaacgggggacggctcaaaagaaatcaacatcgtctggggcatccaggtcccgatattccacaatggcccttgggtctccacgaaccatcctgtgaggtgagaggtacaggatgagagctgagggctcccaaaagggagtctgcaggcggcaacaaagcttgggcgtctgccctcctcacctgttgcgaggtttcccaggataacctccctggcctcggaaggcatcatagttccctcgaccagcaccatacggggcatgggggtatggagggcctcctgtggggactgcagggcggacagcaccagctatgacagagatcagtgttgagttgcaaaactatgtcctcaattccatcctctgttttcttctcccaaagccacacactcaccaagccccttcatctccctcctgtacttacctccatagcccaagatcgggggccggggctgaccatagggcatcaggccctggggagtctggtgtgggtaggggagtcctggggtcaaacctggggggagtacaacacggacagggacatgaattactgcgggggcggggagggggatac

SEQ ID NO: 20 - DNA, 2225 bp, Homo sapiens
ggggtgtgtgcggggggccggaggcggcggctgtcagagtcggctcagcctgcgccggggaacatcggccgcctccagctcccggcgcggcccggcccggcccggctcggccgcctcagacgccgcctgccctgcagccatgaggcccccgcagtgtctgctgcacacgccttccctggcttccccactccttctcctcctcctctggctcctgggtggaggagtgggggctgagggccgggaggatgcagagctgctggtgacggtgcgtgggggccggctgcggggcattcgcctgaagacccccgggggccctgtctctgctttcctgggcatcccctttgcggagccacccatgggaccccgtcgctttctgccaccggagcccaagcagccttggtcaggggtggtagacgctacaaccttccagagtgtctgctaccaatatgtggacaccctatacccaggttttgagggcaccgagatgtggaaccccaaccgtgagctgagcgaggactgcctgtacctcaacgtgtggacaccatacccccggcctacatcccccacccctgtcctcgtctggatctatgggggtggcttctacagtggggcctcctccttggacgtgtacgatggccgcttcttggtacaggccgagaggactgtgctggtgtccatgaactaccgggtgggagcctttggcttcctggccctgccggggagccgagaggccccgggcaatgtgggtctcctggatcagaggctggccctgcagtgggtgcaggagaacgtggcagccttcgggggtgacccgacatcagtgacgctgtttggggagagcgcgggagccgcctcggtgggcatgcacctgctgtccccgcccagccggggcctgttccacagggccgtgctgcagagcggtgcccccaatggaccctgggccacggtgggcatgggagaggcccgtcgcagggccacgcagctggcccaccttgtgggctgtcctccaggcggcactggtgggaatgacacagagctggtagcctgccttcggacacgaccagcgcaggtcctggtgaaccacgaatggcacgtgctgcctcaagaaagcgtcttccggttctccttcgtgcctgtggtagatggagacttcctcagtgacaccccagaggccctcatcaacgcgggagacttccacggcctgcaggtgctggtgggtgtggtgaaggatgagggctcgtattttctggtttacggggccccaggcttcagcaaagacaacgagtctctcatcagccgggccgagttcctggccggggtgcgggtcggggttccccaggtaagtgacctggcagccgaggctgtggtcctgcattacacagactggctgcatcccgaggacccggcacgcctgagggaggccctgagcgatgtggtgggcgaccacaatgtcgtgtgccccgtggcccagctggctgggcgactggctgcccagggtgcccgggtctacgcctacgtctttgaacaccgtgcttccacgctctcctggcccctgtggatgggggtgccccacggctacgagatcgagttcatctttgggatccccctggacccctctcgaaactacacggcagaggagaaaatcttcgcccagcgactgatgcgatactgggccaactttgcccgcacaggggatcccaatgagccccgagaccccaaggccccacaatggcccccgtacacggcgggggctcagcagtacgttagtctggacctgcggccgctggaggtgcggcgggggctgcgcgcccaggcctgcgccttctggaaccgcttcctccccaaattgctcagcgccaccgacacgctcgacgaggcggagcgccagtggaaggccgagttccaccgctggagctcctacatggtgcactggaagaaccagttcgaccactacagcaagcaggatcgctgctcagacctgtgaccccggcgggacccccatgtcctccgctccgcccggccccctagctgtatatactatttatttcagggctgggctataacacagacgagccccagactctgcccatccccaccccaccccgacgtcccccggggctcccggtcctctgcatgtctcaggctgagctccctcccccgcggtgccttcgcccctctgggctgccaataaactgttacagccaaaaaaaaaaaaaaaaaaaaaa

SEQ ID NO: 21 - DNA, 2978 bp, Homo sapiens
ggggtgtgtgcggggggccggaggcggcggctgtcagagtcggctcagcctgcgccggggaacatcggccgcctccagctcccggcgcggcccggcccggcccggctcggccgcctcagacgccgcctgccctgcagccatgaggcccccgcagtgtctgctgcacacgccttccctggcttccccactccttctcctcctcctctggctcctgggtggaggagtgggggctgagggccgggaggatgcagagctgctggtgacggtgcgtgggggccggctgcggggcattcgcctgaagacccccgggggccctgtctctgctttcctgggcatcccctttgcggagccacccatgggaccccgtcgctttctgccaccggagcccaagcagccttggtcaggggtggtagacgctacaaccttccagagtgtctgctaccaatatgtggacaccctatacccaggttttgagggcaccgagatgtggaaccccaaccgtgagctgagcgaggactgcctgtacctcaacgtgtggacaccatacccccggcctacatcccccacccctgtcctcgtctggatctatgggggtggcttctacagtggggcctcctccttggacgtgtacgatggccgcttcttggtacaggccgagaggactgtgctggtgtccatgaactaccgggtgggagcctttggcttcctggccctgccggggagccgagaggccccgggcaatgtgggtctcctggatcagaggctggccctgcagtgggtgcaggagaacgtggcagccttcgggggtgacccgacatcagtgacgctgtttggggagagcgcgggagccgcctcggtgggcatgcacctgctgtccccgcccagccggggcctgttccacagggccgtgctgcagagcggtgcccccaatggaccctgggccacggtgggcatgggagaggcccgtcgcagggccacgcagctggcccaccttgtgggctgtcctccaggcggcactggtgggaatgacacagagctggtagcctgccttcggacacgaccagcgcaggtcctggtgaaccacgaatggcacgtgctgcctcaagaaagcgtcttccggttctccttcgtgcctgtggtagatggagacttcctcagtgacaccccagaggccctcatcaacgcgggagacttccacggcctgcaggtgctggtgggtgtggtgaaggatgagggctcgtattttctggtttacggggccccaggcttcagcaaagacaacgagtctctcatcagccgggccgagttcctggccggggtgcgggtcggggttccccaggtaagtgacctggcagccgaggctgtggtcctgcattacacagactggctgcatcccgaggacccggcacgcctgagggaggccctgagcgatgtggtgggcgaccacaatgtcgtgtgccccgtggcccagctggctgggcgactggctgcccagggtgcccgggtctacgcctacgtctttgaacaccgtgcttccacgctctcctggcccctgtggatgggggtgccccacggctacgagatcgagttcatctttgggatccccctggacccctctcgaaactacacggcagaggagaaaatcttcgcccagcgactgatgcgatactgggccaactttgcccgcacaggggatcccaatgagccccgagaccccaaggccccacaatggcccccgtacacggcgggggctcagcagtacgttagtctggacctgcggccgctggaggtgcggcgggggctgcgcgcccaggcctgcgccttctggaaccgcttcctccccaaattgctcagcgccaccgcctcggaggctcccagcacctgcccaggcttcacccatggggaggctgctccgaggcccggcctccccctgcccctcctcctcctccaccagcttctcctcctcttcctctcccacctccggcggctgtgaacacggcctcttcccctacggccacaggggcccctcctctaatgagtggtcggaccgtggggaagggccccactcagggatctcagacctagtgctcccttcctcctcaaaccgagagactcacactggacagggcaggaggagggggccgtgcctcccacccttctcagggacccccacgcctttgttgtttgaatggaaatggaaaagccagtattcttttataaaattatcttttggaacctgagcctgacattggggggaagtgggaggccccggacggggtagcaccccccattggggctataacggtcaaccatttctgtctcttctttttcccccaacctccccctcctgtcccctctgttcccgtcttccggtcattcttttctcctcctctctccttcctgctgtccttctccggccccgcctctgccctcatcctccctctcgtctttcgcacattctcctgatcctcttgccaccgtcccacgtggtcgcctgcatttctccgtgcgtcctccctgcactcatacccccccttcaacccgcccaaatgtccgatccccgaccttcctcgtgccgtcctcccctcccgcctcgctgggcgccctggccgcagacacgctcgacgaggcggagcgccagtggaaggccgagttccaccgctggagctcctacatggtgcactggaagaaccagttcgaccactacagcaagcaggatcgctgctcagacctgtgaccccggcgggacccccatgtcctccgctccgcccggccccctagctgtatatactatttatttcagggctgggctataacacagacgagccccagactctgcccatccccaccccaccccgacgtcccccggggctcccggtcctctgcatgtctcaggctgagctccctcccccgcggtgccttcgcccctctgggctgccaataaactgttacagccaaaaaaaaaaaaaaaaaaaaaa

SEQ ID NO: 22 - DNA, 85 bp, Homo sapiens
ttggcttgagtccctgagcaaagccactgggaatgctccctgaggacgttatatgagtgctcagctcatggggctatgatggtca

SEQ ID NO: 23 - RNA, 17 nt, Homo sapiens
ucccugagcaaagccac

SEQ ID NO: 24 - RNA, 17 nt, Homo sapiens
caccgaaacgagucccu

SEQ ID NO: 25 - RNA, 16 nt, Homo sapiens
ccacccuucucaggga

SEQ ID NO: 26 - RNA, 86 nt, Homo sapiens
ugccagucucuaggucccugagacccuuuaaccugugaggacauccagggucacaggugagguucuugggagccuggcgucuggcc

SEQ ID NO: 27 - RNA, 24 nt, Homo sapiens
ucccugagacccuuuaaccuguga

SEQ ID NO: 28 - RNA, 24 nt, Homo sapiens
aguguccaauuucccagagucccu

SEQ ID NO: 29 - RNA, 24 nt, Homo sapiens
cgugccucccacccuucucaggga

SEQ ID NO: 30 - RNA, 89 nt, Homo sapiens
accagacuuuuccuagucccugagacccuaacuugugagguauuuuaguaacaucacaagucaggcucuugggaccuaggcggagggga

SEQ ID NO: 31 - RNA, 22 nt, Homo sapiens
ucccugagacccuaacuuguga

SEQ ID NO: 32 - RNA, 22 nt, Homo sapiens
aguguucaaucccagagucccu

SEQ ID NO: 33 - RNA, 22 nt, Homo sapiens
ugccucccacccuucucaggga

SEQ ID NO: 34 - RNA, 80 nt, Homo sapiens
acucugauccuggggcucagcgaguuugcaagggguguuucuguccauggucaggcuugccagccuugguccuugggccc

SEQ ID NO: 35 - RNA, 17 nt, Homo sapiens
uggggcucagcgaguuu

SEQ ID NO: 36 - RNA, 17 nt, Homo sapiens
uuugagcgacucggggu

SEQ ID NO: 37 - RNA, 18 nt, Homo sapiens
uaacacagacgagcccca

SEQ ID NO: 38 - DNA, 59 bp, Homo sapiens
ggaggagcagcagggtgaaactgacacagttctggtgagtttcactttgctgctcctcc

SEQ ID NO: 39 - DNA, 22 bp, Homo sapiens
gcagcagggtgaaactgacaca

SEQ ID NO: 40 - RNA, 22 nt, Homo sapiens
acacagucaaagugggacgacg

SEQ ID NO: 41 - RNA, 22 nt, Homo sapiens
ccuccucucuccuuccugcugu

SEQ ID NO: 42 - RNA, 110 nt, Homo sapiens
ggccuggcuggacagaguugucaugugucugccugucuacacuugcugugcagaacauccgcucaccuguacagcaggcacagacaggcagucacaugacaacccagccu

SEQ ID NO: 43 - RNA, 22 nt, Homo sapiens
acagcaggcacagacaggcagu

SEQ ID NO: 44 - RNA, 22 nt, Homo sapiens
ugacggacagacacggacgaca

SEQ ID NO: 45 - RNA, 23 nt, Homo sapiens
uccuccucucuccuuccugcugu

SEQ ID NO: 46 - RNA, 88 nt, Homo sapiens
ucaggucuucagcagaagcagggagguucucccagugguuuuccuugacugugaggaacuagccugcugcuuugcucaggagugagcu

SEQ ID NO: 47 - RNA, 24 nt, Homo sapiens
agcagaagcagggagguucuccca

SEQ ID NO: 48 - RNA, 24 nt, Homo sapiens
acccucuuggagggacgaagacga

SEQ ID NO: 49 - RNA, 25 nt, Homo sapiens
uuucuccuccucucuccuuccugcu

SEQ ID NO: 50 - DNA, 100 bp, Homo sapiens
gggccaaggtgggccaggggtggtgttgggacagctccgtttaaaaaggcatctccaagagcttccatcaaaggctgcctcttggtgcagcacaggtaga

SEQ ID NO: 51 - RNA, 25 nt, Homo sapiens
ugccucgacaggguuguggugggga

SEQ ID NO: 52 - RNA, 24 nt, Homo sapiens
cagacucugcccauccccacccca

SEQ ID NO: 53 - DNA, 20 bp, Artificial Sequence
  Synthetic
ctgccacgttctcctgcacc

SEQ ID NO: 54 - DNA, 20 bp, Artificial Sequence
  Synthetic
ctgccacgttctcctgcnnn

SEQ ID NO: 55 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 56 - DNA, 1125 bp, Homo sapiens
ttctgccctcgagcccaccgggaacgaaagagaagctctatctcgcctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattc

SEQ ID NO: 57 - protein, 191 aa, Homo sapiens
MQTIKCVVVGDGAVGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQKGLKNVFDEAILAALEPPEPKKSRRCVLL

SEQ ID NO: 58 - DNA, 2272 bp, Homo sapiens
gtcaggtgcgtgcccctgtccggcagccgaggagaccccgcgcagtgctgccaacgccccggtggagaagctgagacggagtctcactgtgttgcccaggctggagtgcagtggcgccatcttggctcactgcagtgcgcctctgccccccgagttcaagcgattctcctgcctcaggctcctgagtagctgggactacaggtcatcatcagatttgaaatatttaaagtggatacaaaactatttcagcaatgcagacaattaagtgtgttgttgtgggcgatggtgctgttggtaaaacatgtctcctgatatcctacacaacaaacaaatttccatcggaatatgtaccgactgtttttgacaactatgcagtcacagttatgattggtggagaaccatatactcttggactttttgatactgcagggcaagaggattatgacagattacgaccgctgagttatccacaaacagatgtatttctagtctgtttttcagtggtctctccatcttcatttgaaaacgtgaaagaaaagtgggtgcctgagataactcaccactgtccaaagactcctttcttgcttgttgggactcaaattgatctcagagatgacccctctactattgagaaacttgccaagaacaaacagaagcctatcactccagagactgctgaaaagctggcccgtgacctgaaggctgtcaagtatgtggagtgttctgcacttacacagaaaggcctaaagaatgtatttgacgaagcaatattggctgccctggagcctccagaaccgaagaagagccgcaggtgtgtgctgctatgaacatctctccagagccctttctgcacagctggtgtcggcatcatactaaaagcaatgtttaaatcaaactaaagattaaaaattaaaattcgtttttgcaataatgacaaatgccctgcacctacccacatgcactcgtgtgagacaaggcccataggtatggccccccccttccccctcccagtactagttaattttgagtaattgtattgtcagaaaagtgattagtactatttttttttgttgtttcaaaaaaaaaatttttgtgtgtgtgtttttttttttttttttttttttttttgttgtttaaaagcaaggcatgcttgtggatgactctgtaacagactaattggaattgttgaagctgctccctggttccactctggagagtaatctgggacatcttagtgttttgttttgtttttttccctcctcttttttttgggggggagtgtgtgtggggtttgttttttagtcttgtttttttaattcattaaccagtggttagcccttaaggggaggaggacggattgattccacattccacttcctagatctagtttagaaaacatgttccccatctggtgctcttaggaaggagtatagtaaatgcctcatttaataacatactcctttttgaaagttgccttttctctccacccttgagtagatccagtatttgatgaaactcatgaaagtgggtggagcccatcttgcccctcctcttttctaggacgcactatatgtgactgtgactttcaaggacatttgtttgccatttgctgatttttttgggaagttaatttctaacttctttcactgataaatgaagaaaagtattgcacctttgaaatgcaccaaatgaattgagtttgtaattaaaaaaatttttttccctttcagtcattgtcttatatgcttagcatagatttgcagctcagtagtatatggtgttcctagaatgcagctgaagacctgttatgtagaggaaatacgaggggtggtgctagaagacagacatctgtggaatgattcacatcctctcaagttaggaggatggaggcctgcttcattaagaagctgggggtagggtgggggtggggagaacacttaacaacatggggaccagtcaggggaatccccttatttctgttttgcatatgaggaaccctagagcagccaggtgaggctctctagtttaataaaaatcatggaaagactcttaatgcagactcttcttaagtgttaatagggattttttcagcttattttggttgcagtttccaatttttaaaaatgttgaggtaatctttcccaccttcccaaacctaattcttgtagatgcattagtgttgaaccaatgctttctcatgtctcaattctttgtatatgcattcttttcagatgtattaaacaaacaaaaacccttcaaaaaaaaaaaaaaa

SEQ ID NO: 59 - RNA, 22 nt, Homo sapiens
gcugguaccgacaucugacaau

SEQ ID NO: 60 - RNA, 31 nt, Homo sapiens
ucgccccucugggcugccaauaaacuguuac

SEQ ID NO: 61 - DNA, 17 bp, Homo sapiens
ggggtggggtggggatg

SEQ ID NO: 62 - DNA, 17 bp, Homo sapiens
ggggtgtggtggggatg

SEQ ID NO: 63 - DNA, 27 bp, watermelon
  misc_feature: primer
ccatccccaccacaccccgacgtcccc